SEQUENCE LISTING (118 sequences)

SEQ ID NO: 1 - protein, 178 aa, Homo sapiens
  REGION 1..178: note = Caveolin-1
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

SEQ ID NO: 2 - protein, 20 aa, synthetic construct
  REGION 1..20: note = Modified Caveolin-1 peptide
DGIWKASFTTFTVTKYWFYR

SEQ ID NO: 3 - protein, 7 aa, synthetic construct
  REGION 1..7: note = Modified Caveolin-1 peptide
FTTFTVT

SEQ ID NO: 4 - protein, 13 aa, synthetic construct
  REGION 1..13: note = Modified Caveolin-1 peptide
KASFTTFTVTKGS

SEQ ID NO: 5 - protein, 13 aa, synthetic construct
  REGION 1..13: note = Modified Caveolin-1 peptide
  MOD_RES 13: note = AMIDATION
KASFTTFTVTKGS

SEQ ID NO: 6 - protein, 19 aa, synthetic construct
  REGION 1..19: note = Modified Caveolin-1 peptide
  SITE 1..2: note = D-Alanine
  SITE 18..19: note = D-Alanine
AAEGKASFTTFTVTKGSAA

SEQ ID NO: 7 - protein, 19 aa, synthetic construct
  REGION 1..19: note = Modified Caveolin-1 peptide
  SITE 1..2: note = D-alanine
  SITE 18..19: note = D-alanine
  MOD_RES 19: note = AMIDATION
AAEGKASFTTFTVTKGSAA

SEQ ID NO: 8 - protein, 19 aa, synthetic construct
  REGION 1..19: note = Modified Caveolin-1 peptide
  MOD_RES 1: note = ACETYLATION
  SITE 1..2: note = D-alanine
  SITE 18..19: note = D-alanine
  MOD_RES 19: note = AMIDATION
AAEGKASFTTFTVTKGSAA

SEQ ID NO: 9 - protein, 12 aa, synthetic construct
  REGION 1..12: note = Modified Caveolin-1 peptide
  SITE 1: note = Orn
  SITE 11: note = Orn
XASFTTFTVTXS

SEQ ID NO: 10 - protein, 12 aa, synthetic construct
  REGION 1..12: note = Modified Caveolin-1 peptide
  SITE 1: note = Orn
  SITE 11: note = Orn
  MOD_RES 11: note = AMIDATION
XASFTTFTVTXS

SEQ ID NO: 11 - protein, 7 aa, synthetic construct
  REGION 1..7: note = Modified Caveolin-1 peptide
  MOD_RES 7: note = AMIDATION
FTTFTVT

SEQ ID NO: 12 - protein, 8 aa, synthetic construct
  REGION 1..8: note = Modified Caveolin-1 peptide
  MOD_RES 8: note = AMIDATION
FTTFTVTK

SEQ ID NO: 13 - protein, 11 aa, synthetic construct
  REGION 1..11: note = Modified Caveolin-1 peptide
  MOD_RES 11: note = AMIDATION
KASFTTFTVTK

SEQ ID NO: 14 - protein, 11 aa, synthetic construct
  REGION 1..11: note = Modified Caveolin-1 peptide
  MOD_RES 1: note = ACETYLATION
  MOD_RES 11: note = AMIDATION
KASFTTFTVTK

SEQ ID NO: 15 - protein, 11 aa, synthetic construct
  REGION 1..11: note = Modified Caveolin-1 peptide
  SITE 1: note = Orn
  MOD_RES 11: note = AMIDATION
XASFTTFTVTK

SEQ ID NO: 16 - protein, 11 aa, synthetic construct
  REGION 1..11: note = Modified Caveolin-1 peptide
  MOD_RES 1: note = ACETYLATION
  SITE 1: note = Orn
  MOD_RES 11: note = AMIDATION
XASFTTFTVTK

SEQ ID NO: 17 - protein, 13 aa, synthetic construct
  REGION 1..13: note = Modified Caveolin-1 peptide
  MOD_RES 1: note = ACETYLATION
  MOD_RES 13: note = AMIDATION
KASFTTFTVTKGS

SEQ ID NO: 18 - protein, 14 aa, synthetic construct
  REGION 1..14: note = Modified Caveolin-1 peptide
  MOD_RES 14: note = AMIDATION
DSGKASFTTFTVTK

SEQ ID NO: 19 - protein, 14 aa, synthetic construct
  REGION 1..14: note = Modified Caveolin-1 peptide
  MOD_RES 1: note = ACETYLATION
  MOD_RES 14: note = AMIDATION
DSGKASFTTFTVTK

SEQ ID NO: 20 - protein, 12 aa, synthetic construct
  REGION 1..12: note = Modified Caveolin-1 peptide
  MOD_RES 1: note = ACETYLATION
  SITE 1: note = Orn
  SITE 11: note = Orn
  MOD_RES 12: note = AMIDATION
XASFTTFTVTXS

SEQ ID NO: 21 - protein, 9 aa, synthetic construct
  REGION 1..9: note = Modified Caveolin-1 peptide
ASFTTFTVT

SEQ ID NO: 22 - protein, 12 aa, synthetic construct
  REGION 1..12: note = Modified Caveolin-1 peptide
KASFTTFTVTKY

SEQ ID NO: 23 - protein, 12 aa, synthetic construct
  REGION 1..12: note = Modified Caveolin-1 peptide
IWKASFTTFTVT

SEQ ID NO: 24 - protein, 13 aa, synthetic construct
  REGION 1..13: note = Modified Caveolin-1 peptide
SFTTFTVTKYWFY

SEQ ID NO: 25 - protein, 13 aa, synthetic construct
  REGION 1..13: note = Modified Caveolin-1 peptide
KASFTTFTVTKYW

SEQ ID NO: 26 - protein, 13 aa, synthetic construct
  REGION 1..13: note = Modified Caveolin-1 peptide
IWKASFTTFTVTK

SEQ ID NO: 27 - protein, 14 aa, synthetic construct
  REGION 1..14: note = Modified Caveolin-1 peptide
FTTFTVTKYWFYRL

SEQ ID NO: 28 - protein, 14 aa, synthetic construct
  REGION 1..14: note = Modified Caveolin-1 peptide
ASFTTFTVTKYWFY

SEQ ID NO: 29 - protein, 14 aa, synthetic construct
  REGION 1..14: note = Modified Caveolin-1 peptide
WKASFTTFTVTKYW

SEQ ID NO: 30 - protein, 14 aa, synthetic construct
  REGION 1..14: note = Modified Caveolin-1 peptide
GIWKASFTTFTVTK

SEQ ID NO: 31 - protein, 15 aa, synthetic construct
  REGION 1..15: note = Modified Caveolin-1 peptide
FTTFTVTKYWFYRLL

SEQ ID NO: 32 - protein, 15 aa, synthetic construct
  REGION 1..15: note = Modified Caveolin-1 peptide
ASFTTFTVTKYWFYR

SEQ ID NO: 33 - protein, 15 aa, synthetic construct
  REGION 1..15: note = Modified Caveolin-1 peptide
WKASFTTFTVTKYWF

SEQ ID NO: 34 - protein, 15 aa, synthetic construct
  REGION 1..15: note = Modified Caveolin-1 peptide
GIWKASFTTFTVTKY

SEQ ID NO: 35 - protein, 15 aa, synthetic construct
  REGION 1..15: note = Modified Caveolin-1 peptide
FDGIWKASFTTFTVT

SEQ ID NO: 36 - protein, 16 aa, synthetic construct
  REGION 1..16: note = Modified Caveolin-1 peptide
SFTTFTVTKYWFYRLL

SEQ ID NO: 37 - protein, 16 aa, synthetic construct
  REGION 1..16: note = Modified Caveolin-1 peptide
KASFTTFTVTKYWFYR

SEQ ID NO: 38 - protein, 16 aa, synthetic construct
  REGION 1..16: note = Modified Caveolin-1 peptide
IWKASFTTFTVTKYWF

SEQ ID NO: 39 - protein, 16 aa, synthetic construct
  REGION 1..16: note = Modified Caveolin-1 peptide
DGIWKASFTTFTVTKY

SEQ ID NO: 40 - protein, 16 aa, synthetic construct
  REGION 1..16: note = Modified Caveolin-1 peptide
SFDGIWKASFTTFTVT

SEQ ID NO: 41 - protein, 17 aa, synthetic construct
  REGION 1..17: note = Modified Caveolin-1 peptide
SFTTFTVTKYWFYRLLS

SEQ ID NO: 42 - protein, 17 aa, synthetic construct
  REGION 1..17: note = Modified Caveolin-1 peptide
KASFTTFTVTKYWFYRL

SEQ ID NO: 43 - protein, 17 aa, synthetic construct
  REGION 1..17: note = Modified Caveolin-1 peptide
IWKASFTTFTVTKYWFY

SEQ ID NO: 44 - protein, 17 aa, synthetic construct
  REGION 1..17: note = Modified Caveolin-1 peptide
DGIWKASFTTFTVTKYW

SEQ ID NO: 45 - protein, 17 aa, synthetic construct
  REGION 1..17: note = Modified Caveolin-1 peptide
SFDGIWKASFTTFTVTK

SEQ ID NO: 46 - protein, 18 aa, synthetic construct
  REGION 1..18: note = Modified Caveolin-1 peptide
FTTFTVTKYWFYRLLSAL

SEQ ID NO: 47 - protein, 18 aa, synthetic construct
  REGION 1..18: note = Modified Caveolin-1 peptide
ASFTTFTVTKYWFYRLLS

SEQ ID NO: 48 - protein, 18 aa, synthetic construct
  REGION 1..18: note = Modified Caveolin-1 peptide
WKASFTTFTVTKYWFYRL

SEQ ID NO: 49 - protein, 18 aa, synthetic construct
  REGION 1..18: note = Modified Caveolin-1 peptide
GIWKASFTTFTVTKYWFY

SEQ ID NO: 50 - protein, 18 aa, synthetic construct
  REGION 1..18: note = Modified Caveolin-1 peptide
FDGIWKASFTTFTVTKYW

SEQ ID NO: 51 - protein, 18 aa, synthetic construct
  REGION 1..18: note = Modified Caveolin-1 peptide
HSFDGIWKASFTTFTVTK

SEQ ID NO: 52 - protein, 19 aa, synthetic construct
  REGION 1..19: note = Modified Caveolin-1 peptide
FTTFTVTKYWFYRLLSALF

SEQ ID NO: 53 - protein, 19 aa, synthetic construct
  REGION 1..19: note = Modified Caveolin-1 peptide
ASFTTFTVTKYWFYRLLSA

SEQ ID NO: 54 - protein, 19 aa, synthetic construct
  REGION 1..19: note = Modified Caveolin-1 peptide
WKASFTTFTVTKYWFYRLL

SEQ ID NO: 55 - protein, 19 aa, synthetic construct
  REGION 1..19: note = Modified Caveolin-1 peptide
GIWKASFTTFTVTKYWFYR

SEQ ID NO: 56 - protein, 19 aa, synthetic construct
  REGION 1..19: note = Modified Caveolin-1 peptide
FDGIWKASFTTFTVTKYWF

SEQ ID NO: 57 - protein, 19 aa, synthetic construct
  REGION 1..19: note = Modified Caveolin-1 peptide
HSFDGIWKASFTTFTVTKY

SEQ ID NO: 58 - protein, 19 aa, synthetic construct
  REGION 1..19: note = Modified Caveolin-1 peptide
GTHSFDGIWKASFTTFTVT

SEQ ID NO: 59 - protein, 8 aa, synthetic construct
  REGION 1..8: note = Modified Caveolin-1 peptide
FTTFTVTK

SEQ ID NO: 60 - protein, 8 aa, synthetic construct
  REGION 1..8: note = Modified Caveolin-1 peptide
SFTTFTVT

SEQ ID NO: 61 - protein, 9 aa, synthetic construct
  REGION 1..9: note = Modified Caveolin-1 peptide
FTTFTVTKY

SEQ ID NO: 62 - protein, 9 aa, synthetic construct
  REGION 1..9: note = Modified Caveolin-1 peptide
SFTTFTVTK

SEQ ID NO: 63 - protein, 9 aa, synthetic construct
  REGION 1..9: note = Modified Caveolin-1 peptide
ASFTTFTVT

SEQ ID NO: 64 - protein, 10 aa, synthetic construct
  REGION 1..10: note = Modified Caveolin-1 peptide
FTTFTVTKYW

SEQ ID NO: 65 - protein, 10 aa, synthetic construct
  REGION 1..10: note = Modified Caveolin-1 peptide
SFTTFTVTKY

SEQ ID NO: 66 - protein, 10 aa, synthetic construct
  REGION 1..10: note = Modified Caveolin-1 peptide
ASFTTFTVTK

SEQ ID NO: 67 - protein, 10 aa, synthetic construct
  REGION 1..10: note = Modified Caveolin-1 peptide
KASFTTFTVT

SEQ ID NO: 68 - protein, 11 aa, synthetic construct
  REGION 1..11: note = Modified Caveolin-1 peptide
FTTFTVTKYWF

SEQ ID NO: 69 - protein, 11 aa, synthetic construct
  REGION 1..11: note = Modified Caveolin-1 peptide
SFTTFTVTKYW

SEQ ID NO: 70 - protein, 11 aa, synthetic construct
  REGION 1..11: note = Modified Caveolin-1 peptide
ASFTTFTVTKY

SEQ ID NO: 71 - protein, 11 aa, synthetic construct
  REGION 1..11: note = Modified Caveolin-1 peptide
KASFTTFTVTK

SEQ ID NO: 72 - protein, 11 aa, synthetic construct
  REGION 1..11: note = Modified Caveolin-1 peptide
WKASFTTFTVT

SEQ ID NO: 73 - protein, 12 aa, synthetic construct
  REGION 1..12: note = Modified Caveolin-1 peptide
FTTFTVTKYWFY

SEQ ID NO: 74 - protein, 12 aa, synthetic construct
  REGION 1..12: note = Modified Caveolin-1 peptide
SFTTFTVTKYWF

SEQ ID NO: 75 - protein, 12 aa, synthetic construct
  REGION 1..12: note = Modified Caveolin-1 peptide
ASFTTFTVTKYW

SEQ ID NO: 76 - protein, 12 aa, synthetic construct
  REGION 1..12: note = Modified Caveolin-1 peptide
WKASFTTFTVTK

SEQ ID NO: 77 - protein, 13 aa, synthetic construct
  REGION 1..13: note = Modified Caveolin-1 peptide
FTTFTVTKYWFYR

SEQ ID NO: 78 - protein, 13 aa, synthetic construct
  REGION 1..13: note = Modified Caveolin-1 peptide
ASFTTFTVTKYWF

SEQ ID NO: 79 - protein, 13 aa, synthetic construct
  REGION 1..13: note = Modified Caveolin-1 peptide
WKASFTTFTVTKY

SEQ ID NO: 80 - protein, 13 aa, synthetic construct
  REGION 1..13: note = Modified Caveolin-1 peptide
GIWKASFTTFTVT

SEQ ID NO: 81 - protein, 14 aa, synthetic construct
  REGION 1..14: note = Modified Caveolin-1 peptide
SFTTFTVTKYWFYR

SEQ ID NO: 82 - protein, 14 aa, synthetic construct
  REGION 1..14: note = Modified Caveolin-1 peptide
KASFTTFTVTKYWF

SEQ ID NO: 83 - protein, 14 aa, synthetic construct
  REGION 1..14: note = Modified Caveolin-1 peptide
IWKASFTTFTVTKY

SEQ ID NO: 84 - protein, 14 aa, synthetic construct
  REGION 1..14: note = Modified Caveolin-1 peptide
DGIWKASFTTFTVT

SEQ ID NO: 85 - protein, 15 aa, synthetic construct
  REGION 1..15: note = Modified Caveolin-1 peptide
SFTTFTVTKYWFYRL

SEQ ID NO: 86 - protein, 15 aa, synthetic construct
  REGION 1..15: note = Modified Caveolin-1 peptide
KASFTTFTVTKYWFY

SEQ ID NO: 87 - protein, 15 aa, synthetic construct
  REGION 1..15: note = Modified Caveolin-1 peptide
IWKASFTTFTVTKYW

SEQ ID NO: 88 - protein, 15 aa, synthetic construct
  REGION 1..15: note = Modified Caveolin-1 peptide
DGIWKASFTTFTVTK

SEQ ID NO: 89 - protein, 16 aa, synthetic construct
  REGION 1..16: note = Modified Caveolin-1 peptide
FTTFTVTKYWFYRLLS

SEQ ID NO: 90 - protein, 16 aa, synthetic construct
  REGION 1..16: note = Modified Caveolin-1 peptide
ASFTTFTVTKYWFYRL

SEQ ID NO: 91 - protein, 16 aa, synthetic construct
  REGION 1..16: note = Modified Caveolin-1 peptide
WKASFTTFTVTKYWFY

SEQ ID NO: 92 - protein, 16 aa, synthetic construct
  REGION 1..16: note = Modified Caveolin-1 peptide
GIWKASFTTFTVTKYW

SEQ ID NO: 93 - protein, 16 aa, synthetic construct
  REGION 1..16: note = Modified Caveolin-1 peptide
FDGIWKASFTTFTVTK

SEQ ID NO: 94 - protein, 17 aa, synthetic construct
  REGION 1..17: note = Modified Caveolin-1 peptide
FTTFTVTKYWFYRLLSA

SEQ ID NO: 95 - protein, 17 aa, synthetic construct
  REGION 1..17: note = Modified Caveolin-1 peptide
ASFTTFTVTKYWFYRLL

SEQ ID NO: 96 - protein, 17 aa, synthetic construct
  REGION 1..17: note = Modified Caveolin-1 peptide
WKASFTTFTVTKYWFYR

SEQ ID NO: 97 - protein, 17 aa, synthetic construct
  REGION 1..17: note = Modified Caveolin-1 peptide
GIWKASFTTFTVTKYWF

SEQ ID NO: 98 - protein, 17 aa, synthetic construct
  REGION 1..17: note = Modified Caveolin-1 peptide
FDGIWKASFTTFTVTKY

SEQ ID NO: 99 - protein, 17 aa, synthetic construct
  REGION 1..17: note = Modified Caveolin-1 peptide
HSFDGIWKASFTTFTVT

SEQ ID NO: 100 - protein, 18 aa, synthetic construct
  REGION 1..18: note = Modified Caveolin-1 peptide
SFTTFTVTKYWFYRLLSA

SEQ ID NO: 101 - protein, 18 aa, synthetic construct
  REGION 1..18: note = Modified Caveolin-1 peptide
KASFTTFTVTKYWFYRLL

SEQ ID NO: 102 - protein, 18 aa, synthetic construct
  REGION 1..18: note = Modified Caveolin-1 peptide
IWKASFTTFTVTKYWFYR

SEQ ID NO: 103 - protein, 18 aa, synthetic construct
  REGION 1..18: note = Modified Caveolin-1 peptide
DGIWKASFTTFTVTKYWF

SEQ ID NO: 104 - protein, 18 aa, synthetic construct
  REGION 1..18: note = Modified Caveolin-1 peptide
SFDGIWKASFTTFTVTKY

SEQ ID NO: 105 - protein, 18 aa, synthetic construct
  REGION 1..18: note = Modified Caveolin-1 peptide
THSFDGIWKASFTTFTVT

SEQ ID NO: 106 - protein, 19 aa, synthetic construct
  REGION 1..19: note = Modified Caveolin-1 peptide
SFTTFTVTKYWFYRLLSAL

SEQ ID NO: 107 - protein, 19 aa, synthetic construct
  REGION 1..19: note = Modified Caveolin-1 peptide
KASFTTFTVTKYWFYRLLS

SEQ ID NO: 108 - protein, 19 aa, synthetic construct
  REGION 1..19: note = Modified Caveolin-1 peptide
IWKASFTTFTVTKYWFYRL

SEQ ID NO: 109 - protein, 19 aa, synthetic construct
  REGION 1..19: note = Modified Caveolin-1 peptide
DGIWKASFTTFTVTKYWFY

SEQ ID NO: 110 - protein, 19 aa, synthetic construct
  REGION 1..19: note = Modified Caveolin-1 peptide
SFDGIWKASFTTFTVTKYW

SEQ ID NO: 111 - protein, 19 aa, synthetic construct
  REGION 1..19: note = Modified Caveolin-1 peptide
THSFDGIWKASFTTFTVTK

SEQ ID NO: 112 - protein, 13 aa, synthetic construct
  REGION 1..13: note = Cell penetrating peptide
GRKKRRQRRRPPQ

SEQ ID NO: 113 - protein, 16 aa, synthetic construct
  REGION 1..16: note = Cell penetrating peptide
RQIKIWFQNRRMKWKK

SEQ ID NO: 114 - protein, 26 aa, synthetic construct
  REGION 1..26: note = Cell penetrating peptide
GIGAVLKVLTTGLPALISWIKRKRQQ

SEQ ID NO: 115 - protein, 10 aa, synthetic construct
  REGION 1..10: note = Cell penetrating peptide
TKIESLKEHG

SEQ ID NO: 116 - protein, 10 aa, synthetic construct
  REGION 1..10: note = Cell penetrating peptide
TQIENLKEKG

SEQ ID NO: 117 - protein, 26 aa, synthetic construct
  REGION 1..26: note = Cell penetrating peptide
AALEALAEALEALAEALEALAEAAAA

SEQ ID NO: 118 - protein, 25 aa, synthetic construct
  REGION 1..25: note = Cell penetrating peptide
GLFEAIEGFIENGWEGMIEGWYGCG

(19) United States
(12) Patent Application Publication
(10) Pub. No.: US 2025/0262273 A1
(43) Pub. Date: Aug. 21, 2025

(54) MODIFIED CAVEOLIN-1 PEPTIDES FOR THE TREATMENT OF CHRONIC KIDNEY DISEASE

(71) Applicant: Rein Therapeutics, Inc., Austin, TX (US)

(72) Inventors: BreAnne MACKENZIE, Austin, TX (US); Cory HOGABOAM, Los Angeles, CA (US); Brian WINDSOR, Austin, TX (US)

(21) Appl. No.: 18/702,462

(22) PCT Filed: Oct. 21, 2022
(86) PCT No.: PCT/US2022/078482

§ 371 Date: Apr. 18, 2024

Related U.S. Application Data
(60) Provisional application No. 63/270,852, filed on Oct. 22, 2021.

(51) Int. Cl. A61K 38/17; A61K 9/00; A61K 31/427; A61K 31/4706; A61K 31/4965; A61K 31/513; A61K 31/706; A61P 11/00; A61P 13/12

(52) U.S. Cl. CPC A61K 38/177; A61K 9/0019; A61K 9/0075; A61K 31/427; A61K 31/4706; A61K 31/4965; A61K 31/513; A61K 31/706; A61P 11/00; A61P 13/12

(57) ABSTRACT

Provided herein are methods of using modified caveolin-1 (Cav-1) peptides to treat or prevent a kidney disease or disorder in a subject. In particular, provided are methods of using the modified Cav-1 peptides for the treatment of chronic kidney disease characterized by fibrosis, such as, for example, Alport syndrome.

### CROSS REFERENCE TO RELATED APPLICATIONS

This application claims the benefit of priority to U.S. Provisional Application No. 63/270,852 filed on Oct. 22, 2021, the content of which is incorporated by reference in its entirety.

### FIELD OF THE DISCLOSURE

The present disclosure generally relates to the fields of molecular biology and medicine. In particular, the present disclosure provides compositions comprising modified caveolin-1 peptides for the treatment of chronic kidney disease. The present disclosure also provides compositions comprising modified caveolin-1 peptides for the treatment of fibrotic diseases in elderly subjects.

### REFERENCE TO AN ELECTRONIC SEQUENCE LISTING

The contents of the electronic sequence listing (LUTX_024_01WO_SeqList_ST26.xml; Size: 162,055 bytes; and Date of Creation: Oct. 11, 2022) is herein incorporated by reference in its entirety.

### BACKGROUND

Chronic kidney disease represents a worldwide health concern affecting more than 20 million Americans and about 10% of the global population. In chronic kidney disease, the progressive loss of renal function occurs as a consequence of the deposition of fibrous tissue between the functional units of the kidney or nephrons as well as the ongoing replacement of the filtration surface by fibrous tissue. Kidney fibrosis is a pathological hallmark of chronic kidney disease and a major contributing factor of progression to end-stage renal disease in which the individual requires routine dialysis or a kidney transplant in order to survive.

Clinical management of chronic kidney disease focuses primarily on controlling blood pressure using renin-angiotensin system (RAS) inhibitors, which slows disease progression, but this treatment is not curative. Therefore, there remains a need in the art for alternative therapies that target fibrotic processes in the kidney that may slow or halt progression of chronic kidney disease.

### BRIEF SUMMARY

The present disclosure provides a method of treating or preventing a kidney disease or disorder in a subject comprising administering to the subject an effective amount of a modified Cav-1 peptide: (a) consisting of any one of the amino acid sequences of SEQ ID NOs: 2-111; (b) comprising any one of the amino acid sequences of SEQ ID NOs: 2-111; or (c) comprising any one of the amino acid sequences of SEQ ID NOs: 2-111 with one or more amino acid substitutions, insertions, deletions, or chemical modifications.

In some embodiments, the modified Cav-1 peptide comprises L-amino acids. In some embodiments, the modified Cav-1 peptide comprises D-amino acids. In some embodiments, the modified Cav-1 peptide comprises both L- and D-amino acids. In some embodiments, the modified Cav-1 peptide comprises deuterated residues. In some embodiments, the modified Cav-1 peptide comprises at least one non-standard amino acid. In some embodiments, the non-standard amino acid is ornithine.

In some embodiments, the modified Cav-1 peptide comprises an N-terminal modification. In some embodiments, the modified Cav-1 peptide comprises a C-terminal modification. In some embodiments, the modified Cav-1 peptide comprises an N-terminal modification and a C-terminal modification. In some embodiments, the N-terminal modification is acylation. In some embodiments, the C-terminal modification is amidation.

In some embodiments, the modified Cav-1 peptide comprises the amino acid sequence of FTTFTVT (SEQ ID NO: 3). In some embodiments, the modified Cav-1 peptide comprises the amino acid sequence of KASFTTFTVTKGS (SEQ ID NO: 4). In some embodiments, the modified Cav-1 peptide comprises the amino acid sequence of KASFTTFTVTKGS-NH2 (SEQ ID NO: 5). In some embodiments, the modified Cav-1 peptide comprises the amino acid sequence of aaEGKASFTTFTVTKGSaa (SEQ ID NO: 6). In some embodiments, the modified Cav-1 peptide comprises the amino acid sequence of aaEGKASFTTFTVTKGSaa-NH2 (SEQ ID NO: 7). In some embodiments, the modified Cav-1 peptide comprises the amino acid sequence of Ac-aaEGKASFTTFTVTKGSaa-NH2 (SEQ ID NO: 8). In some embodiments, the modified Cav-1 peptide comprises the amino acid sequence of OASFTTFTVTOS (SEQ ID NO: 9). In some embodiments, the modified Cav-1 peptide comprises the amino acid sequence of OASFTTFTVTOS-NH2 (SEQ ID NO: 10).

In some embodiments, the modified Cav-1 peptide comprises an internalization sequence. In some embodiments, the internalization sequence is located at the C-terminal end of the peptide. In some embodiments, the internalization sequence is located at the N-terminal end of the peptide.

In some embodiments, the modified Cav-1 peptide further comprises a cap at the N- and/or C-terminus. In some embodiments, the modified Cav-1 peptide comprises the cap at the N-terminus and C-terminus.

In some embodiments, the modified Cav-1 peptide is cyclized.

In some embodiments, the modified Cav-1 peptide maintains the biological activity of native Cav-1 (SEQ ID NO: 1).

In some embodiments, the modified Cav-1 peptide is a multimer comprising at least two peptides described herein. In some embodiments, a first peptide of the at least two peptides is essentially identical to a second peptide of the at least two peptides. In some embodiments, a first peptide of the at least two peptides is not identical to a second peptide of the at least two peptides.

In some embodiments, the modified Cav-1 peptide is administered intravenously, subcutaneously, intramuscularly, intraperitoneally, or orally. In some embodiments, the modified Cav-1 peptide is administered subcutaneously. In some embodiments, the modified Cav-1 peptide is administered intravenously.

In some embodiments, the kidney disease or disorder is selected from the group consisting of chronic kidney disease, end-stage renal disease, glomerulonephritis, focal segmental glomerulosclerosis, kidney fibrosis, polycystic kidney disease, IgA nephropathy, lupus nephritis, nephrotic syndrome, Alport syndrome, amyloidosis, Goodpasture syndrome, granulomatosis with polyangiitis, or acute kidney injury. In some embodiments, the kidney disease or disorder is Alport syndrome. In some embodiments, the kidney disease or disorder is characterized by fibrosis.

In some embodiments, the method further comprises administering an effective amount of at least one additional therapeutic agent. In some embodiments, the at least one additional therapeutic agent is an angiotensin-converting enzyme (ACE) inhibitor and/or angiotensin II receptor (ARB) inhibitor. In some embodiments, the method further comprises treating the subject with dialysis.

In some embodiments, the modified Cav-1 peptide is administered as a composition comprising the modified Cav-1 peptide and at least one pharmaceutically acceptable carrier or excipient.

A method of treating or preventing a fibrotic disease or disorder in an elderly subject comprising administering to the subject an effective amount of a modified Cav-1 peptide: (a) consisting of any one of the amino acid sequences of SEQ ID NOs: 2-111; (b) comprising any one of the amino acid sequences of SEQ ID NOs: 2-111; or (c) comprising any one of the amino acid sequences of SEQ ID NOs: 2-111 with one or more amino acid substitutions, insertions, deletions, or chemical modifications. In some embodiments, the modified Cav-1 peptide comprises the amino acid sequence of FTTFTVT (SEQ ID NO: 3). In some embodiments, the modified Cav-1 peptide comprises the amino acid sequence of Ac-aaEGKASFTTFTVTKGSaa-NH2 (SEQ ID NO: 8). In some embodiments, the fibrotic disease or disorder is interstitial lung disease. In some embodiments, the interstitial lung disease is idiopathic pulmonary fibrosis.

In some embodiments, the modified Cav-1 peptide is administered to the lung. In some embodiments, the modified Cav-1 peptide is administered to the lung via inhalation. In some embodiments, the modified Cav-1 peptide is administered to the subject using a nebulizer. In some embodiments, the modified Cav-1 peptide is administered to the subject using an inhaler.

In some embodiments, the modified Cav-1 peptide is formulated for inhalation. In some embodiments, the modified Cav-1 peptide is formulated for pressurized metered dose inhalation. In some embodiments, the modified Cav-1 peptide is formulated as a dry powder. In some embodiments, the dry powder comprising the modified Cav-1 peptide is essentially excipient free. In some embodiments, the dry powder is produced by a spray-drying process, air jet milling, ball milling, or wet milling. In some embodiments, the modified Cav-1 peptide is formulated for nebulization.

In some embodiments, the method further comprises administering to the subject a therapeutically effective amount of at least one additional therapeutic agent. In some embodiments, the at least one additional therapeutic agent is chloroquine, hydroxychloroquine, remdesivir, favipiravir, lopinavir, or ritonavir.

In some embodiments, the subject is at least 55 years old, at least 60 years old, at least 65 years old, at least 70 years old, at least 75 years old, at least 80 years old, at least 85 years old, or at least 90 years old.

### BRIEF DESCRIPTION OF THE DRAWINGS

FIG. 1A shows a urinary protein gel from Cohort 1 and Cohort 2 Col4a3−/− mice prior to treatment with saline or APi2355 peptide. Urine was loaded onto the gel using creatinine level as a calibrator. Cohort 1 started treatment at 6 weeks-old and Cohort 2 started treatment at 5 weeks-old. Mouse ID Nos. 1046 and 1117 died during treatment for unknown reasons. Asterisks indicates mouse albumin.

FIG. 1B shows a urinary protein gel from Cohort 1 and Cohort 2 Col4a3−/− mice after 2 weeks of daily intraperitoneal injections with APi2355 peptide or saline. Cohort 1 started treatment at 6 weeks-old and Cohort 2 started treatment at 5 weeks-old. Asterisks indicates mouse albumin.

FIG. 1C shows albumin to creatinine ratios (g/mg) in the urine of Cohort 1 (left panel) and Cohort 2 (right panel) Col4a3−/− mice before and after 2 weeks of treatment with saline or APi2355 peptide. Cohort 1 started treatment at 6 weeks-old and Cohort 2 started treatment at 5 weeks-old.

FIG. 2 shows blood urea nitrogen (BUN) levels of Cohort 1 (left panel) and Cohort 2 (right panel) Col4a3−/− mice following treatment with saline or APi2355 peptide. Cohort 1 started treatment at 6 weeks-old and Cohort 2 started treatment at 5 weeks-old.

FIG. 3A shows immunofluorescence staining of collagen I and nidogen on kidney tissue from an untreated Col4a3+/− mouse (left panel) or Col4a3−/− mice following 2 weeks of treatment with saline (middle panel) or APi2355 peptide (right panel). Mice started treatment at 6 weeks-old.

FIG. 3B shows immunofluorescence staining of collagen I and nidogen on kidney tissue from an untreated Col4a3+/− mouse (left panel) or Col4a3−/− mice following 2 weeks of treatment with saline (middle panel) or APi2355 peptide (right panel). Mice started treatment at 6 weeks-old.

FIG. 4A shows immunofluorescence staining of collagen I and α smooth muscle actin (αSMA) on kidney tissue from an untreated Col4a3+/− mouse (left panel) or Col4a3−/− mice following 2 weeks of treatment with saline (middle panel) or APi2355 peptide (right panel). Mice started treatment at 6 weeks-old.

FIG. 4B shows immunofluorescence staining of collagen I and α smooth muscle actin (αSMA) on kidney tissue from an untreated Col4a3+/− mouse (left panel) or Col4a3−/− mice following 2 weeks of treatment with saline (middle panel) or APi2355 peptide (right panel). Mice started treatment at 6 weeks-old.

FIG. 5A shows immunofluorescence staining of collagen I and nidogen on kidney tissue from an untreated Col4a3+/− mouse (left panel) or Col4a3−/− mice following 2 weeks of treatment with saline (middle panel) or APi2355 peptide (right panel). Mice started treatment at 5 weeks-old.

FIG. 5B shows immunofluorescence staining of collagen I and nidogen on kidney tissue from an untreated Col4a3+/− mouse (left panel) or Col4a3−/− mice following 2 weeks of treatment with saline (middle panel) or APi2355 peptide (right panel). Mice started treatment at 5 weeks-old.

FIG. 6A shows immunofluorescence staining of collagen I and α smooth muscle actin (αSMA) on kidney tissue from an untreated Col4a3+/− mouse (left panel) or Col4a3−/− mice following 2 weeks of treatment with saline (middle panel) or APi2355 peptide (right panel). Mice started treatment at 5 weeks-old.

FIG. 6B shows immunofluorescence staining of collagen I and α smooth muscle actin (αSMA) on kidney tissue from an untreated Col4a3+/− mouse (left panel) or Col4a3−/− mice following 2 weeks of treatment with saline (middle panel) or APi2355 peptide (right panel). Mice started treatment at 5 weeks-old.

FIG. 7A shows a urinary protein gel from 4 week-old Col4a3−/− mice prior to treatment with saline or APi2355 peptide. Urine was loaded onto the gel using creatinine level as a calibrator. Asterisks indicates mouse albumin.

FIG. 7B shows a urinary protein gel from 6 week-old female (F) and male (M) Col4a3−/− mice after 2 weeks of daily intraperitoneal injections with APi2355 peptide or normal saline. Asterisks indicates mouse albumin.

FIG. 7C shows a urinary protein gel from 7 and 8 week-old female (F) and male (M) Col4a3−/− mice after 3 or 4 weeks of daily intraperitoneal injections with APi2355 peptide or normal saline. Asterisks indicates mouse albumin.

FIG. 7D shows albumin to creatinine ratios (g/mg) in the urine of Col4a3−/− mice before treatment at 4 weeks-old and during treatment with saline or APi2355 peptide at 6, 7, and 8 weeks-old.

FIG. 8 shows blood urea nitrogen (BUN) levels of Col4a3−/− mice before treatment at 4 weeks-old and during treatment with saline or APi2355 peptide at 6 and 8 weeks-old.

FIG. 9A shows immunofluorescence staining of collagen I and nidogen on kidney tissue from an untreated male Col4a3+/− mouse.

FIG. 9B shows immunofluorescence staining of collagen I and nidogen on kidney tissue from a female Col4a3−/− mouse treated daily with APi2355 peptide for 4 weeks.

FIG. 9C shows immunofluorescence staining of collagen I and nidogen on kidney tissue from a female Col4a3−/− mouse treated daily with normal saline for 4 weeks.

FIG. 9D shows immunofluorescence staining of collagen I and nidogen on kidney tissue from a female Col4a3−/− mouse treated daily with APi2355 peptide for 4 weeks.

FIG. 9E shows immunofluorescence staining of collagen I and nidogen on kidney tissue from a female Col4a3−/− mouse treated daily with APi2355 peptide for 4 weeks.

FIG. 9F shows immunofluorescence staining of collagen I and nidogen on kidney tissue from a female Col4a3−/− mouse treated daily with saline for 4 weeks.

FIG. 9G shows immunofluorescence staining of collagen I and nidogen on kidney tissue from a male Col4a3−/− mouse treated daily with APi2355 peptide for 4 weeks.

FIG. 9H shows immunofluorescence staining of collagen I and nidogen on kidney tissue from a male Col4a3−/− mouse treated daily with APi2355 peptide for 4 weeks.

FIG. 10A shows immunofluorescence staining of caveolin-1 and laminin-111 (LM111) on intestine tissue from an untreated Col4a3+/− mouse (left panel) or Col4a3−/− mice following 4 weeks of treatment with saline (middle panel) or APi2355 peptide (right panel).

FIG. 10B shows immunofluorescence staining of caveolin-1 and laminin-111 (LM111) on intestine tissue from an untreated Col4a3+/− mouse (left panel) or Col4a3−/− mice following 4 weeks of treatment with saline (middle panel) or APi2355 peptide (right panel).

FIG. 10C shows immunofluorescence staining of caveolin-1 and laminin-111 (LM111) on kidney tissue from an untreated Col4a3+/− mouse (left panel) or Col4a3−/− mice following 4 weeks of treatment with saline (middle panel) or APi2355 peptide (right panel).

FIG. 10D shows immunofluorescence staining of caveolin-1 and laminin-111 (LM111) on kidney tissue from an untreated Col4a3+/− mouse (left panel) or Col4a3−/− mice following 4 weeks of treatment with saline (middle panel) or APi2355 peptide (right panel).

FIG. 10E shows immunofluorescence staining of caveolin-1 and laminin-111 (LM111) on kidney tissue from an untreated Col4a3+/− mouse (left panel) or Col4a3−/− mice following 4 weeks of treatment with saline (middle panel) or APi2355 peptide (right panel).

FIG. 10F shows immunofluorescence staining of caveolin-1 and laminin-111 (LM111) on kidney tissue from an untreated Col4a3+/− mouse (left panel) or Col4a3−/− mice following 4 weeks of treatment with saline (middle panel) or APi2355 peptide (right panel).

FIG. 11A shows immunofluorescence staining of caveolin-1, collagen I, laminin-111, and nidogen on kidney tissue from untreated Col4a3+/− and Col4a3−/− mice or Col4a3−/− mice following 4 weeks of treatment with saline or APi2355 peptide.

FIG. 11B shows quantification of glomerular fibrosis in 8 week-old female Col4a3−/− mice following 4 weeks of treatment with normal saline or APi2355 peptide.

FIG. 12 shows collagen in total lung homogenates of saline (SAL)- or bleomycin (BLM)-treated aged mice administered control peptide (CP) or CSP-7 through dry powder inhalation (DPI).

FIG. 13 shows collagen in total lung homogenates of saline (SAL)- or bleomycin (BLM)-treated aged mice administered control peptide (CP) or CSP-7 through dry powder inhalation (DPI). ****P<0.0001.

FIG. 14A shows total SMAD (tSMAD, top panel) and phosphorylated SMAD (pSMAD, bottom panel) in total lung homogenates of bleomycin (BLM)-treated aged mice either untreated or administered control peptide (CP) or CSP-7 through dry powder inhalation or CSP-7 through intraperitoneal injection (IP).

FIG. 14B shows galectin 7 in total lung homogenates of bleomycin (BLM)-treated aged mice either untreated or administered control peptide (CP) or CSP-7 through dry powder inhalation or CSP-7 through intraperitoneal injection (IP).

FIG. 15A shows immunohistochemistry staining for caveolin-1 (Cav-1) in kidney tissue from normal donor A and normal donor B at 5× and 63× magnification. Arrows indicate positive Cav-1 staining in glomeruli of normal kidney tissue.

FIG. 15B shows immunohistochemistry staining for caveolin-1 (Cav-1) in fibrotic kidney tissue from patient C, patient D, and patient E at 5× and 63× magnification. Arrows indicate glomeruli in fibrotic kidney tissue.

### DETAILED DESCRIPTION

The present disclosure provides modified caveolin-1 (Cav-1) peptides and uses thereof for the treatment or prevention of chronic kidney disease in a subject. Also provided herein are methods of treating a fibrotic disease or disorder in an elderly subject by administering the modified Cav-1 peptide or pharmaceutical composition thereof.

### I. Definitions

As used herein, the articles “a” or “an” refers to one or more than one of the grammatical object of the article. As used herein in the claim(s), when used in conjunction with the word “comprising,” the articles “a” or “an” refer to one or more than one of the grammatical object of the article.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

The terms “peptide”, “polypeptide” or “protein” is used in its broadest sense to refer to a molecule of two or more amino acids, amino acid analogs, or peptidomimetics. In some embodiments, the amino acids are linked by peptide bonds. In some embodiments, the amino acids are linked by other types of bonds, e.g. ester, ether, etc. As used herein, the term “amino acid” refers to either natural and/or unnatural or synthetic amino acids, including glycine and both the D or L optical isomers, and amino acid analogs and peptidomimetics.

The term “peptidomimetic” or “peptide mimic” refers to a peptide modified in such a way that it includes at least one non-peptidic bond such as, for example, urea bond, carbamate bond, sulfonamide bond, hydrazine bond, or any other covalent bond.

In some embodiments, a polypeptide or polypeptide has a certain percentage (for example, 80%, 85%, 90%, or 95%) of “sequence identity” or “homology” to another sequence meaning that, when aligned, that percentage of amino acids are the same in comparing the two sequences. In some embodiments, the term “identity” or “homology” refers to the percentage of amino acid residues in the candidate sequence that are identical with the residue of a corresponding sequence to which it is compared after aligning the sequences and introducing gaps, if necessary, to achieve the maximum percent identity for the entire sequence. Alignment and the percent homology or sequence identity can be determined using software programs known in the art, for example those described in Current Protocols In Molecular Biology (F. M. Ausubel et al., eds., 1987) Supplement 30, section 7.7.18, Table 7.7.1.

The term “substantially pure” refers to a peptide or polypeptide that has been isolated and purified to at least some degree from the components that naturally accompany it. Typically, a peptide or polypeptide is substantially pure when it is at least about 60%, at least about 70%, at least about 80%, at least about 90%, at least about 95%, or at least about 99%, by weight, free from the proteins and naturally-occurring organic molecules with which it is naturally associated. For example, a substantially pure peptide or polypeptide may be obtained by extraction from a natural source, by expression of a recombinant nucleic acid in a cell that does not normally express that protein, or by chemical synthesis.

The term “isolated”, as used herein, refers to a peptide or polypeptide that has been separated from any natural environment, such as a body fluid, e.g., blood, and separated from the components that naturally accompany the peptide.

As used herein, “essentially free,” in terms of a specified component, means that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected using standard analytical methods.

The term “variant” as used herein refers to a polypeptide or nucleic acid that differs from the reference polypeptide or nucleic acid by one or more amino acid or nucleic acid deletions, additions, substitutions or side-chain modifications, yet retains one or more specific functions or biological activities of the naturally occurring molecule. Also encompassed within the term variant is a polynucleotide or polypeptide that can vary in primary, secondary, or tertiary structure, as compared to a reference polynucleotide or polypeptide, respectively (e.g., as compared to a wild-type polynucleotide or polypeptide).

The term “insertion” refers to the addition of one or more amino acids within a peptide or polypeptide sequence and the term “deletion” refers to the removal of one or more amino acids within a peptide or polypeptide sequence. “Insertions” and “deletions” are typically in the range of about 1 to 5 amino acids. The variation can be experimentally determined by producing the peptide synthetically while systematically making insertions, deletions, or substitutions of nucleotides in the sequence using recombinant DNA techniques.

The term “substitution” when referring to a peptide or polypeptide, refers to a change in an amino acid for a different entity, for example another amino acid or amino acid moiety. Amino acid substitutions include alterations in which an amino acid is replaced with a different naturally occurring or a non-conventional amino acid residue. Such substitutions may be classified as “conservative,” in which case an amino acid residue contained in a polypeptide is replaced with another naturally occurring amino acid of similar character either in relation to polarity, side chain functionality or size. Such conservative substitutions are well known in the art. Substitutions encompassed by the present invention may also be “non-conservative,” in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments, amino acid substitutions are conservative. In some embodiments, the amino acid substitutions are non-conservative.

An “analog” refers to a molecule, such as a peptide, that is similar in function to either the entire molecule or a fragment thereof. The term “analog” is also intended to include allelic species and induced variants. Analogs typically differ from naturally occurring peptides at one or more amino acid residues, often by virtue of conservative amino acid substitutions. Analogs typically exhibit at least about 80% or at least about 90% sequence identity with natural peptides. Some analogs also include unnatural amino acids or modifications of N- or C-terminal amino acids. Examples of unnatural amino acids include, but are not limited to disubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, and σ-N-methylarginine. Fragments and analogs can be screened for prophylactic or therapeutic efficacy using in vitro and in vivo methods well known to those skilled in the art.

The term “covalently bonded” refers to a peptide or polypeptide joined either directly or indirectly (e.g., through a linker) by a covalent chemical bond. In some embodiments, fusion peptides of the present disclosure are covalently bonded.

The term “fusion protein” as used herein refers to a recombinant protein of two or more proteins. Fusion proteins can be produced, for example, by joining a nucleic acid sequence encoding one protein to the nucleic acid encoding another protein such that they constitute a single open-reading frame that can be translated in host cells into a single polypeptide harboring all the intended proteins. The order of arrangement of the proteins can vary. Fusion proteins can include an epitope tag or a half-life extender. Epitope tags include biotin, FLAG tag, c-myc, hemaglutinin, His6, digoxigenin, FITC, Cy3, Cy5, green fluorescent protein, V5 epitope tags, GST, β-galactosidase, AU1, AU5, and avidin. Half-life extenders include Fc domain and serum albumin.

The term “airway”, as used herein, refers to any portion of the respiratory tract including the upper respiratory tract, the respiratory airway, and the lungs. The upper respiratory tract includes the nose and nasal passages, mouth, and throat. The respiratory airway includes the larynx, trachea, bronchi and bronchioles. The lungs include the respiratory bronchioles, alveolar ducts, alveolar sacs and alveoli.

The terms “nebulizing,” “nebulized” and other grammatical variations used herein, refer to the process of converting a liquid into small aerosol droplets using a nebulizer.

The term “air jet mill” refers to a device or method for reducing particle size by using a jet of compressed gas to impact particles into one another, thereby pulverizing the particles. In some embodiments, an air jet mill is used to reduce the size of peptide particles. Other mechanical milling devices that perform the same function can also be used interchangeably with the air jet mill. Air jet milling can occur under various environmental parameters such as temperature, pressure, relative/absolution humidity, oxygen content, etc.

The term “ball mill” refers to a device or method for reducing particle size by adding the particle of interest and a grinding medium to the interior of a cylinder and rotating the cylinder. The particles of interest are broken down as the grinding medium rises and falls along the exterior of the cylinder as it rotates. In some embodiments, a ball mill is used to reduce the size of peptide particles. Other mechanical milling devices that perform the same function can also be used interchangeably with the air jet mill.

The term “wet mill” or “media mill” refers to a device or method for reducing particle size by adding the particle of interest to device with an agitator, containing a media comprising a liquid and a grinding medium. With the addition of the particle of interest, as the agitator rotates, the energy it disperses causes the grinding medium and particles of interest to come into contact and break down the particles of interest. Other mechanical milling devices that perform the same function can also be used interchangeably with the air jet mill.

The term “high pressure homogenization” refers to a method of reducing particle size by adding the particle of interest to a device which combines both pressure and mechanical forces to break down the particle of interest. Mechanical forces used in high pressure homogenization may include impact, shear, and cavitation, among others. Other mechanical milling devices that perform the same function can also be used interchangeably with the air jet mill.

The term “cryogenic mill” refers to a device or method for reducing particle size by first chilling a particle of interest with dry ice, liquid nitrogen, or other cryogenic liquid, and subsequently milling the particle of interest to reduce the size. Other mechanical milling devices that perform the same function can also be used interchangeably with the air jet mill.

The phrase “effective amount” or “therapeutically effective amount” is an amount effective for treating and/or preventing a disease or disorder, as disclosed herein. In some embodiments, an effective amount is an amount or dose of a composition (e.g., a pharmaceutical composition, compound, or agent) that produces at least one desired therapeutic effect in a subject, such as preventing or treating a target condition or beneficially alleviating a symptom associated with the condition. The most desirable effective amount is an amount that will produce a desired efficacy of a particular treatment selected by one of skill in the art for a given subject in need thereof. This amount will vary depending upon a variety of factors understood by the skilled worker, including but not limited to the characteristics of the composition (including activity, pharmacokinetics, pharmacodynamics, and bioavailability), the physiological condition of the subject (including age, sex, disease type, disease stage, general physical condition, responsiveness to a given dosage, and type of medication), the nature of the pharmaceutically acceptable carrier or carriers in the formulation, and the route of administration.

The term “pharmaceutical composition” or “pharmaceutically acceptable composition” refer to compositions that do not produce an adverse, allergic, or other untoward reaction when administered to a subject. The preparation of a pharmaceutical composition comprising a modified Cav-1 peptide, such as CSP-7, or additional active ingredients will be known to those of skill in the art. Moreover, for animal (e.g., human) administration, it will be understood that preparations should meet bioburden, sterility, pyrogenicity, general safety, and/or purity standards as required by the FDA or other recognized regulatory authority.

As used herein, the term “pharmaceutically acceptable carrier” includes any and all excipients, processing aids, aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, disintegration agents, lubricants, flavor modifiers (e.g., sweetening agents, flavoring agents), such like materials and combinations thereof, as would be known to one of ordinary skill in the art. The pH and exact concentration of the various components in a pharmaceutical composition are adjusted according to well-known parameters. In some embodiments, the carrier may encapsulate a therapeutic agent, but not itself be consumed or administered to a subject (e.g., a shell capsule encasing a dry powder composition, such as for use in a dry powder inhaler). See, e.g., Remington's Pharmaceutical Sciences, 18th Ed., 1990, incorporated herein by reference.

As used herein, “excipient” refers to pharmaceutically acceptable carriers that are relatively inert substances used to facilitate administration or delivery of an Active Pharmaceutical Ingredient (API) (e.g., a modified Cav-1 peptide) into a subject or used to facilitate processing of an API into drug formulations that can be used pharmaceutically for delivery to the site of action in a subject. Excipients or pharmaceutically acceptable carriers include all of the inactive components of the dosage form except for the active ingredient(s). Non-limiting examples of excipients include carrier agents, bulking agents, stabilizing agents, surfactants, surface modifiers, solubility enhancers, buffers, encapsulating agents, antioxidants, preservatives, nonionic wetting or clarifying agents, viscosity increasing agents, and absorption-enhancing agents. The term “excipient free” refers to a modified Cav-1 peptide or pharmaceutical composition thereof in a formulation free of any excipients.

A “biologically active” caveolin-1 (Cav-1) peptide refers to a peptide that increases p53 protein levels, reduces urokinase plasminogen activator (uPA) and uPA receptor (uPAR), and/or increases plasminogen activator inhibitor-1 (PAI-1) expression in cells, such as fibrotic fibroblasts. In some embodiments, the biologically active peptide has at least 20% of the biological or biochemical activity of native Cav-1 polypeptide of SEQ ID NO: 1 (e.g., as measured by an in vitro or an in vivo assay). In some embodiments, the biological active peptide has an increase biological or biochemical activity as compared to the native Cav-1 polypeptide.

The terms “subject”, “individual”, and “patient” are used interchangeably herein, and refer to an animal, for example a human or non-human animal (e.g., a mammal), to whom treatment, including prophylactic treatment, with a modified Cav-1 peptide or pharmaceutical composition thereof as disclosed herein, is provided. The term “subject” as used herein refers to human and non-human animals. The term “non-human animals” includes all vertebrates, e.g., mammals, such as non-human primates (particularly higher primates), sheep, dogs, rodents (e.g., mouse or rat), guinea pigs, goats, pigs, cats, rabbits, cows, and non-mammals such as chickens, amphibians, reptiles. In some embodiments, the subject is human. In some embodiments, the subject is an experimental animal or animal substitute as a disease model. Non-human mammals include mammals such as non-human primates (particularly higher primates), sheep, dogs, rodents (e.g., mouse or rat), guinea pigs, goats, pigs, cats, rabbits and cows. In some embodiments, the non-human animal is a companion animal such as a dog or a cat.

As used herein, the terms “treat,” “treating,” or “treatment”, and grammatical variants thereof, have the same meaning as commonly understood by those of ordinary skill in the art. In some embodiments, these terms may refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (e.g., not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject (e.g., a human) in need of treatment may thus be a subject already afflicted with the disease or disorder in question. The terms “treat,” “treating,” or “treatment” includes inhibition or reduction of an increase in severity of a pathological state or symptoms relative to the absence of treatment, and is not necessarily meant to imply complete cessation of the relevant disease or condition.

As used herein, the terms “prevent,” “preventing,” “prevention” and grammatical variants thereof refer to an approach for preventing the development of, or altering the pathology of, a condition or disease. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g., a human) in need of prevention may thus be a subject not yet afflicted with the disease or disorder in question. The term “prevention” includes slowing the onset of disease relative to the absence of treatment, and is not necessarily meant to imply permanent prevention of the relevant disease, disorder or condition. Thus “preventing” or “prevention” of a condition may in certain contexts refer to reducing the risk of developing the condition, or preventing or delaying the development of symptoms associated with the condition.

### II. Caveolin-1 Peptides

Embodiments of the present disclosure provide modified versions of the native caveolin-1 (Cav-1) protein, including, but not limited to, fragments, derivatives, and variants of the native Cav-1 protein. In some embodiments, the modified Cav-1 peptides are truncations of the native Cav-1 polypeptide, such as the exemplary peptides shown in Table 2 and/or Table 3.

Native human Cav-1 is 178 amino acids in length (see, SEQ ID NO: 1 in Table 1 below) and has a molecular weight of 22 kDa. Caveolin-1 is an integral membrane protein associated with endocytosis, extracellular matrix organization, cholesterol distribution, cell migration, and signaling. See, Boscher and Nabi, Adv Exp Med Biol, 2012; 729-29-50.

TABLE 1
Amino Acid Sequences of Native Human Cav-1 and Cav-1 Scaffolding Domain

| Name | Amino acid sequence | SEQ ID NO |
|---|---|---|
| Native human Cav-1 | MSGGKYVDSEGHLYTVPIREQGNIYKPNNK | 1 |
| | AMADELSEKQVYDAHTKEIDLVNRDPKHLN | |
| | DDVVKIDFEDVIAEPEGTHSFDGIWKASFTTF | |
| | TVTKYWFYRLLSALFGIPMALIWGIYFAILSF | |
| | LHIWAVVPCIKSFLIEIQCISRVYSIYVHTVCD | |
| | PLFEAVGKIFSNVRINLQKEI | |
| Cav-1 scaffolding | DGIWKASFTTFTVTKYWFYR | 2 |
| domain | | |

In some embodiments, the modified Cav-1 peptide is the Cav-1 scaffolding domain (CSD). The CSD is comprised of the amino acids 82-101 of caveolin-1 (see, SEQ ID NO: 2 in Table 1 above). The CSD of caveolin-1 plays a critical role in caveolin-1 dimerization as well as regulation of diverse signaling intermediates (Shetty et al., Am J Respir Cell Mol Biol 2012; 47:474-83; Fridolfsson et al., FASEB J 2014; 28:3823-31; Degryse et al., Am J Physiol Cell Mol Physiol 2010; 299: LA42-L452; and Egger et al., PLos One, 2013; 8:e63432). The CSD domain of caveolin-1 has demonstrated inhibition of Wnt-signaling, β-catenin-mediated transcription, activation of SRC, EGFR, MEK1 and ERK-2 and various other factors (see, Shetty et al., Am J Respir Cell Mol Biol 2012; 47:474-83; Bhandary et al., Am J Physiol Cell Mol Physiol 2012; 302: LA63-L473; Bhandary et al., Am J Pathol 2013; 183:131-143; Fridolfsson et al., FASEB J 2014; 28:3823-31; Degryse et al., Am J Physiol Cell Mol Physiol 2010; 299: L442-L452; and Fiddler et al., Ann Am Thorac Soc, 2016; 13:1430-2). For example, the CSD of Cav-1 interferes with Cav-1 interaction with SRC kinases and mimics the combined effect of uPA and anti-β1-integrin antibody. Endogenous CSD domains can form homodimers with other Cav-1 proteins and interact with proteins that have a caveolin binding domain sequence (CBD) motif. It is estimated that up to 30% of all endogenous proteins have CBD motifs and the caveolin-1 CSD domain is hypothesized to provide stability to these proteins (see, Marudamuthu et al., Am J Pathol 2015; 185:55-68).

In some embodiments, the modified Cav-1 peptide is CSP-7. CSP-7 is a seven amino acid fragment of the human CSD of caveolin-1 (see, SEQ ID NO: 3 in Table 3 below).

Exemplary amino acid sequences of the modified Cav-1 peptides are shown below in Tables 2 and 3. Upper case letters denote L-amino acids and lower case letters denote D-amino acids (e.g., lowercase “a” represents D-alanine). The term “Ac” refers to an acetyl group and the term “NH2” refers to an amido group. The “O” denotes ornithine.

TABLE 2
Illustrative Modified Cav-1 Peptides

| Amino Acid Sequence | SEQ ID NO |
|---|---|
| KASFTTFTVTKGS | 4 |
| KASFTTFTVTKGS-NH2 | 5 |
| aaEGKASFTTFTVTKGSaa | 6 |
| aaEGKASFTTFTVTKGSaa-NH2 | 7 |
| Ac-aaEGKASFTTFTVTKGSaa-NH2 | 8 |
| OASFTTFTVTOS | 9 |
| OASFTTFTVTOS-NH2 | 10 |
| FTTFTVT-NH2 | 11 |
| FTTFTVTK-NH2 | 12 |
| KASFTTFTVTK-NH2 | 13 |
| Ac-KASFTTFTVTK-NH2 | 14 |
| OASFTTFTVTK-NH2 | 15 |
| Ac-OASFTTFTVTK-NH2 | 16 |
| Ac-KASFTTFTVTKGS-NH2 | 17 |
| DSGKASFTTFTVTK-NH2 | 18 |
| Ac-DSGKASFTTFTVTK-NH2 | 19 |
| Ac-OASFTTFTVTOS-NH2 | 20 |

TABLE 3
Additional Illustrative Modified Cav-1 Peptides

| Amino acid sequence | SEQ ID NO | Amino acid sequence | SEQ ID NO |
|---|---|---|---|
| FTTFTVT | 3 | ASFTTFTVTK | 66 |
| ASFTTFTVT | 21 | KASFTTFTVT | 67 |
| KASFTTFTVTKY | 22 | FTTFTVTKYWF | 68 |
| IWKASFTTFTVT | 23 | SFTTFTVTKYW | 69 |
| SFTTFTVTKYWFY | 24 | ASFTTFTVTKY | 70 |
| KASFTTFTVTKYW | 25 | KASFTTFTVTK | 71 |
| IWKASFTTFTVTK | 26 | WKASFTTFTVT | 72 |
| FTTFTVTKYWFYRL | 27 | FTTFTVTKYWFY | 73 |
| ASFTTFTVTKYWFY | 28 | SFTTFTVTKYWF | 74 |
| WKASFTTFTVTKYW | 29 | ASFTTFTVTKYW | 75 |
| GIWKASFTTFTVTK | 30 | WKASFTTFTVTK | 76 |
| FTTFTVTKYWFYRLL | 31 | FTTFTVTKYWFYR | 77 |
| ASFTTFTVTKYWFYR | 32 | ASFTTFTVTKYWF | 78 |
| WKASFTTFTVTKYWF | 33 | WKASFTTFTVTKY | 79 |
| GIWKASFTTFTVTKY | 34 | GIWKASFTTFTVT | 80 |
| FDGIWKASFTTFTVT | 35 | SFTTFTVTKYWFYR | 81 |
| SFTTFTVTKYWFYRLL | 36 | KASFTTFTVTKYWF | 82 |
| KASFTTFTVTKYWFYR | 37 | IWKASFTTFTVTKY | 83 |
| IWKASFTTFTVTKYWF | 38 | DGIWKASFTTFTVT | 84 |
| DGIWKASFTTFTVTKY | 39 | SFTTFTVTKYWFYRL | 85 |
| SFDGIWKASFTTFTVT | 40 | KASFTTFTVTKYWFY | 86 |
| SFTTFTVTKYWFYRLLS | 41 | IWKASFTTFTVTKYW | 87 |
| KASFTTFTVTKYWFYRL | 42 | DGIWKASFTTFTVTK | 88 |
| IWKASFTTFTVTKYWFY | 43 | FTTFTVTKYWFYRLLS | 89 |
| DGIWKASFTTFTVTKYW | 44 | ASFTTFTVTKYWFYRL | 90 |
| SFDGIWKASFTTFTVTK | 45 | WKASFTTFTVTKYWFY | 91 |
| FTTFTVTKYWFYRLLSAL | 46 | GIWKASFTTFTVTKYW | 92 |
| ASFTTFTVTKYWFYRLLS | 47 | FDGIWKASFTTFTVTK | 93 |
| WKASFTTFTVTKYWFYRL | 48 | FTTFTVTKYWFYRLLSA | 94 |
| GIWKASFTTFTVTKYWFY | 49 | ASFTTFTVTKYWFYRLL | 95 |
| FDGIWKASFTTFTVTKYW | 50 | WKASFTTFTVTKYWFYR | 96 |
| HSFDGIWKASFTTFTVTK | 51 | GIWKASFTTFTVTKYWF | 97 |
| FTTFTVTKYWFYRLLSALF | 52 | FDGIWKASFTTFTVTKY | 98 |
| ASFTTFTVTKYWFYRLLSA | 53 | HSFDGIWKASFTTFTVT | 99 |
| WKASFTTFTVTKYWFYRLL | 54 | SFTTFTVTKYWFYRLLSA | 100 |
| GIWKASFTTFTVTKYWFYR | 55 | KASFTTFTVTKYWFYRLL | 101 |
| FDGIWKASFTTFTVTKYWF | 56 | IWKASFTTFTVTKYWFYR | 102 |
| HSFDGIWKASFTTFTVTKY | 57 | DGIWKASFTTFTVTKYWF | 103 |
| GTHSFDGIWKASFTTFTVT | 58 | SFDGIWKASFTTFTVTKY | 104 |
| FTTFTVTK | 59 | THSFDGIWKASFTTFTVT | 105 |
| SFTTFTVT | 60 | SFTTFTVTKYWFYRLLSAL | 106 |
| FTTFTVTKY | 61 | KASFTTFTVTKYWFYRLLS | 107 |
| SFTTFTVTK | 62 | IWKASFTTFTVTKYWFYRL | 108 |
| ASFTTFTVT | 63 | DGIWKASFTTFTVTKYWFY | 109 |
| FTTFTVTKYW | 64 | SFDGIWKASFTTFTVTKYW | 110 |
| SFTTFTVTKY | 65 | THSFDGIWKASFTTFTVTK | 111 |

In some embodiments, the Cav-1 peptide or the modified Cav-1 peptide:
(a) consists of any one of the amino acid sequences of SEQ ID NOs: 2-111;
(b) comprises a core sequence of any one of the amino acid sequences of SEQ ID NOs: 2-111; or
(c) comprises a core sequence of any one of the amino acid sequences of SEQ ID NOs: 2-111, wherein the core sequence includes one or more amino acid substitutions, insertions, deletions, or chemical modifications.

In some embodiments, the Cav-1 peptide or the modified Cav-1 peptide comprises a core sequence of any one of the amino acid sequences of SEQ ID NO: 2-111. In some embodiments, the Cav-1 peptide or the modified Cav-1 peptide comprises a core sequence of any one of the amino acid sequences of SEQ ID NO: 2-111, wherein the core sequence includes one or more amino acid substitutions, insertions, deletions, or chemical modifications. In some embodiments, the core sequence is SEQ ID NO: 3. In some embodiments, the core sequence is SEQ ID NO: 6.

In some embodiments, the Cav-1 polypeptide comprises or consists of the amino acid sequence of SEQ ID NO: 1. In some embodiments, the Cav-1 polypeptide comprises an amino acid sequence with at least 40%, at least 50%, at least 60%, at least 70%, at least 80%, at least 85%, at least 90%, at least 95%, at least 96%, at least 97%, at least 98%, or at least 99% sequence identity to SEQ ID NO: 1. In some embodiments, the Cav-1 polypeptide comprises the amino acid sequence of SEQ ID NO: 1 with one or more mutations relative thereto. For example, in some embodiments, the Cav-1 polypeptide comprises 1, 2, 3, 4, 5, 6, 7, 8, 9, 10, or more mutations relative to SEQ ID NO: 1. In some embodiments, the Cav-1 polypeptide comprises the amino acid sequence of SEQ ID NO: 1 with 1-5, 5-10, 11-5, 15-20, 10-25, 25-30, or more than 30 mutations.

In some embodiments, the modified Cav-1 peptide comprises or consists of the amino acid sequence of any one of SEQ ID NOs: 2-111. In some embodiments, the modified Cav-1 peptide comprises an amino acid sequence with at least 40%, at least 50%, at least 60%, at least 70%, at least 80%, at least 85%, at least 90%, at least 95%, at least 96%, at least 97%, at least 98%, or at least 99% sequence identity to any one of SEQ ID NOs: 2-111. In some embodiments, the modified Cav-1 peptide comprises the amino acid sequence of any one of SEQ ID NOs: 2-111 with one or more mutations relative thereto. For example, in some embodiments, the modified Cav-1 peptide comprises 1, 2, 3, 4, 5, 6, 7, 8, 9, 10, or more mutations relative to any one of SEQ ID NOs: 2-111. In some embodiments, the modified Cav-1 peptide comprises the amino acid sequence of any one of SEQ ID NOs: 2-111 with 1-5, 5-10, or 11-15, or more mutations. In some embodiments, the modified Cav-1 peptide comprises an additional 1-5 amino acids at either the N- or C-terminus or at both termini of any one of SEQ ID NOs: 2-111.

In some embodiments, the modified Cav-1 peptide comprises or consists of the amino acid sequence of any one of SEQ ID NOs: 4-20. In some embodiments, the modified Cav-1 peptide comprises an amino acid sequence with at least 40%, at least 50%, at least 60%, at least 70%, at least 80%, at least 85%, at least 90%, at least 95%, at least 96%, at least 97%, at least 98%, or at least 99% sequence identity to any one of SEQ ID NOs: 4-20. In some embodiments, the modified Cav-1 peptide comprises the amino acid sequence of any one of SEQ ID NOs: 4-20 with one or more mutations relative thereto. For example, in some embodiments, the modified Cav-1 peptide comprises 1, 2, 3, 4, 5, 6, 7, 8, 9, 10, or more mutations relative to any one of SEQ ID NOs: 4-20. In some embodiments, the modified Cav-1 peptide comprises the amino acid sequence of any one of SEQ ID NOs: 4-20 with 1-5, 5-10, or 11-15, or more mutations. In some embodiments, the modified Cav-1 peptide comprises an additional 1-5 amino acids at either the N- or C-terminus or at both termini of any one of SEQ ID NOs: 4-20.

In some embodiments, the modified Cav-1 peptide comprises or consists of the amino acid sequence of SEQ ID NO: 2. In some embodiments, the modified Cav-1 peptide comprises an amino acid sequence with at least 40%, at least 50%, at least 60%, at least 70%, at least 80%, at least 85%, at least 90%, at least 91%, at least 92%, at least 93%, at least 94%, or at least 95% sequence identity to SEQ ID NO: 2. In some embodiments, the modified Cav-1 peptide comprises the amino acid sequence of SEQ ID NO: 2 with one or more mutations relative thereto. For example, in some embodiments, the modified Cav-1 peptide comprises 1, 2, 3, 4, 5, 6, 7, 8, 9, 10, or more mutations relative to SEQ ID NO: 2. In some embodiments, the modified Cav-1 peptide comprises the amino acid sequence of SEQ ID NO: 2 with 1-5, 5-10, or 11-15, or more mutations. In some embodiments, the modified Cav-1 peptide comprises an additional 1-5 amino acids at either the N- or C-terminus or at both termini of SEQ ID NO: 2. In some embodiments, the modified Cav-1 peptide of SEQ ID NO: 2 comprises an N- and/or C-terminal modification. In some embodiments, the N-terminal modification is acylation. In some embodiments, the C-terminal modification is amidation.

In some embodiments, the modified Cav-1 peptide comprises or consists of the amino acid sequence of SEQ ID NO: 3. In some embodiments, the modified Cav-1 peptide comprises an amino acid sequence with at least 40%, at least 50%, at least 60%, at least 70%, at least 80%, or at least 85% sequence identity to SEQ ID NO: 3. In some embodiments, the modified Cav-1 peptide comprises the amino acid sequence of SEQ ID NO: 3 with one or more mutations relative thereto. For example, in some embodiments, the modified Cav-1 peptide comprises 1, 2, 3, 4, or 5 mutations relative to SEQ ID NO: 3. In some embodiments, the modified Cav-1 peptide comprises an additional 1-5 amino acids at either the N- or C-terminus or at both termini of SEQ ID NO: 3. In some embodiments, the modified Cav-1 peptide of SEQ ID NO: 3 comprises an N- and/or C-terminal modification. In some embodiments, the N-terminal modification is acylation. In some embodiments, the C-terminal modification is amidation.

In some embodiments, the modified Cav-1 peptide comprises or consists of the amino acid sequence of SEQ ID NO: 4. In some embodiments, the modified Cav-1 peptide comprises an amino acid sequence with at least 40%, at least 50%, at least 60%, at least 70%, at least 80%, at least 85%, at least 90%, at least 91%, or at least 92% sequence identity to SEQ ID NO: 4. In some embodiments, the modified Cav-1 peptide comprises the amino acid sequence of SEQ ID NO: 4 with one or more mutations relative thereto. For example, in some embodiments, the modified Cav-1 peptide comprises 1, 2, 3, 4, 5, 6, 7, 8, 9, 10, or more mutations relative to SEQ ID NO: 4. In some embodiments, the modified Cav-1 peptide comprises the amino acid sequence of SEQ ID NO: 4 with 1-5, 5-10, or more mutations. In some embodiments, the modified Cav-1 peptide comprises an additional 1-5 amino acids at either the N- or C-terminus or at both termini of SEQ ID NO: 4. In some embodiments, the modified Cav-1 peptide of SEQ ID NO: 4 comprises an N- and/or C-terminal modification. In some embodiments, the N-terminal modification is acylation. In some embodiments, the C-terminal modification is amidation. In some embodiments, the modified Cav-1 peptide comprises or consists of the amino acid sequence of SEQ ID NO: 5.

In some embodiments, the modified Cav-1 peptide comprises or consists of the amino acid sequence of SEQ ID NO: 6. In some embodiments, the modified Cav-1 peptide comprises an amino acid sequence with at least 40%, at least 50%, at least 60%, at least 70%, at least 80%, at least 85%, at least 90%, at least 91%, at least 92%, at least 93%, at least 94%, or at least 95% sequence identity to SEQ ID NO: 6. In some embodiments, the modified Cav-1 peptide comprises the amino acid sequence of SEQ ID NO: 6 with one or more mutations relative thereto. For example, in some embodiments, the modified Cav-1 peptide comprises 1, 2, 3, 4, 5, 6, 7, 8, 9, 10, or more mutations relative to SEQ ID NO: 6. In some embodiments, the modified Cav-1 peptide comprises the amino acid sequence of SEQ ID NO: 6 with 1-5, 5-10, or 11-15, or more mutations. In some embodiments, the modified Cav-1 peptide comprises an additional 1-5 amino acids at either the N- or C-terminus or at both termini of SEQ ID NO: 6. In some embodiments, the modified Cav-1 peptide of SEQ ID NO: 6 comprises an N- and/or C-terminal modification. In some embodiments, the N-terminal modification is acylation. In some embodiments, the C-terminal modification is amidation. In some embodiments, the modified Cav-1 peptide comprises the amino acid sequence of SEQ ID NO: 7. In some embodiments, the modified Cav-1 peptide comprises or consists of the amino acid sequence of SEQ ID NO: 8.

In some embodiments, the modified Cav-1 peptide comprises or consists of the amino acid sequence of SEQ ID NO: 9. In some embodiments, the modified Cav-1 peptide comprises an amino acid sequence with at least 40%, at least 50%, at least 60%, at least 70%, at least 80%, at least 85%, at least 90%, at least 91%, or at least 92% sequence identity to SEQ ID NO: 9. In some embodiments, the modified Cav-1 peptide comprises the amino acid sequence of SEQ ID NO: 9 with one or more mutations relative thereto. For example, in some embodiments, the modified Cav-1 peptide comprises 1, 2, 3, 4, 5, 6, 7, 8, 9, 10, or more mutations relative to SEQ ID NO: 9. In some embodiments, the modified Cav-1 peptide comprises the amino acid sequence of SEQ ID NO: 9 with 1-5, 5-10, or more mutations. In some embodiments, the modified Cav-1 peptide comprises an additional 1-5 amino acids at either the N- or C-terminus or at both termini of SEQ ID NO: 9. In some embodiments, the modified Cav-1 peptide of SEQ ID NO: 9 comprises an N- and/or C-terminal modification. In some embodiments, the N-terminal modification is acylation. In some embodiments, the C-terminal modification is amidation. In some embodiments, the modified Cav-1 peptide comprises or consists of the amino acid sequence of SEQ ID NO: 10.

In some embodiments, the modified Cav-1 peptide comprises 1, 2, 3, 4 or more amino acid substitutions, deletions, or insertions relative to the sequence of SEQ ID NO: 1, such as to derive a polypeptide of 3, 4, 5, 6, 7, 8, 9, 10, 11, 12, 13, 14, 15, 16, 17, 18, or 19 residues.

In some embodiments, the modified Cav-1 peptide has the amino acid sequence of FTTFTVT (SEQ ID NO: 3), and 1 to 5 additional amino acids on the N- and/or C-terminus. In some embodiments, the modified Cav-1 peptide has the amino acid sequence of FTTFTVT (SEQ ID NO: 3), and optionally, 1 to 5 additional amino acids on the N- and/or C-terminus.

In some embodiments, the modified Cav-1 peptides provided in the present disclosure exhibit similar or the same biological activity of the native Cav-1 polypeptide in in vitro or in vivo assays. In some embodiments, the modified Cav-1 peptide exhibits at least about 20%, at least about 30%, at least about 40%, at least about 50%, at least about 60%, at least about 65%, at least about 70%, at least about 75%, at least about 80%, at least about 85%, at least about 90%, at least about 95%, at least about 97%, at least about 99%, and any range derivable therein, such as, for example, from about 70% to about 80%, and more preferably from about 81% to about 90%; or even more preferably, from about 91% to about 99% of the activity of the native Cav-1 polypeptide. In some embodiments, the modified Cav-1 peptide has 100% or even greater activity than the native Cav-1 polypeptide. Assays for testing biological activity, e.g., anti-fibrotic activity, the ability to affect expression of uPA, uPAR and PAI-1 mRNAs, or inhibit proliferation of fibroblasts, are well-known in the art.

In some embodiments, the modified Cav-1 peptides of the present disclosure are fragments, derivatives, or variants of the native Cav-1 polypeptide. The peptides can be synthetic, recombinant, or chemically modified peptides isolated or generated using methods well known in the art. Modifications can be made to amino acids on the N-terminus, C-terminus, or internally. Peptides can include conservative or non-conservative amino acid changes, as described below. Polynucleotide changes can result in amino acid substitutions, additions, deletions, fusions and truncations in the Cav-1 polypeptide encoded by the reference sequence. Peptides can also include insertions, deletions or substitutions of amino acids, including insertions and substitutions of amino acids (and other molecules) that do not normally occur in the peptide sequence that is the basis of the modified variant, for example but not limited to, insertion of L-amino acids, or non-standard amino acids such as ornithine, which do not normally occur in human proteins.

### A. Substitutions

In some embodiments, the modified Cav-1 peptide comprises one or more conservative amino acid substitutions. Conservative amino acid substitutions result from replacing one amino acid with another having similar structural and/or chemical properties, such as the replacement of a leucine with an isoleucine or valine, an aspartate with a glutamate, or a threonine with a serine. Thus, a conservative substitution of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide. Conservative substitution tables providing functionally similar amino acids are well known in the art. For example, the following six groups each contain amino acids that are conservative substitutions for one another: 1) Alanine (A), Serine(S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). In some embodiments, individual substitutions, deletions, or additions that alter, add, or delete a single amino acid or a small percentage of amino acids can also be considered conservative substitutions if the change does not reduce the activity of the peptide. Insertions or deletions are typically in the range of about 1 to 6 amino acids.

In some embodiments, one can select the amino acid that will substitute an existing amino acid based on the location of the existing amino acid, such as, for example, wherein the amino acid is exposed to solvents or is present on the outer surface of the peptide or polypeptide as compared to internally localized amino acids not exposed to solvents. Selection of such conservative amino acid substitutions are well known in the art, for example, as disclosed in Dordo et al, J. Mol Biol, 1999, 217, 721-739 and Taylor et al, J. Theor. Biol. 119 (1986); 205-218 and S. French and B. Robson, J. Mol. Evol. 19 (1983) 171. For example, the following conservative amino acid substitutions suitable for the exterior of a peptide or polypeptide can be used: substitution of Y with F; T with S or K; P with A; E with D or Q; N with D or G; R with K; G with N or A; T with S or K; D with N or E; I with L or V; F with Y; S with T or A; R with K; G with N or A; K with R; A with S, K or P.

In some embodiments, one can select conservative amino acid substitutions suitable for the interior of a peptide or polypeptide, such as, for example, wherein the amino acids are not exposed to a solvent. For example, the following conservative amino acid substitutions for the interior of a peptide or polypeptide can be used: where Y is substituted with F; T with A or S; I with L or V; W with Y; M with L; N with D; G with A; T with A or S; D with N; I with L or V; F with Y or L; S with A or T; and A with S, G, T or V. In some embodiments, non-conservative amino acid substitutions are also encompassed within the term of variants.

In some embodiments, an amino acid substitution can be made in a polypeptide at one or more positions wherein the substitution is for an amino acid having a similar hydrophilicity. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Thus, such conservative substitutions can be made in a polypeptide and will likely only have minor effects on their activity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5+1); alanine (0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4), and these values can be used as a guide. Thus, any of the modified Cav-1 peptides described herein may be modified by the substitution of an amino acid, for a different, but homologous amino acid with a similar hydrophilicity value. Amino acids with hydrophilicities within +/−1.0 points, or +/−0.5 points, are considered homologous. In some embodiments, the modified Cav-1 peptide comprises a substitution of amino acids whose hydrophilicity values are within ±2. In some embodiments, the modified Cav-1 peptide comprises a substitution of amino acids whose hydrophilicity values are within ±1. In some embodiments, the modified Cav-1 peptide comprises a substitution of amino acids whose hydrophilicity values are within ±0.5.

In some embodiments, the modified Cav-1 peptide comprises non-naturally occurring amino acids. In some embodiments, the modified Cav-1 peptide comprises a combination of naturally occurring and non-naturally occurring amino acids, or comprises only non-naturally occurring amino acids. The non-naturally occurring amino acids can include synthetic non-native amino acids, substituted amino acids, or one or more D-amino acids (or other components of the composition, with exception for protease recognition sequences) as desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes, and prolonged intravascular and interstitial lifetimes when such properties are desirable. Additionally, D-peptides cannot be processed efficiently for major histocompatibility complex class II-restricted presentation to T helper cells and are therefore less likely to induce humoral immune responses in the whole organism.

In addition to the 20 “standard” L-amino acids, D-amino acids or non-standard, modified or unusual amino acids, which are well-defined in the art, are also contemplated for use in the present disclosure. Phosphorylated amino acids (Ser, Thr, Tyr), glycosylated amino acids (Ser, Thr, Asn), β-amino acids, GABA, ω-amino acids are further contemplated for use in the present disclosure. These include, for example, include β-alanine (β-Ala) and other ω-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); norleucine (Nle); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); homoarginine (hArg); N-acetyl lysine (AcLys); 2,4-diaminobutyric acid (Dbu; Dab); p-aminophenylalanine (Phe(pNH₂)); N-methyl valine (MeVal); homocysteine (hCys), homophenylalanine (hPhe), and homoserine (hSer); hydroxyproline (Hyp), homoproline (hPro), N-methylated amino acids, and peptoids (N-substituted glycines).

### B. Derivatives

In some embodiments, the modified Cav-1 peptides are derivatives of the native Cav-1 polypeptide. The term “derivative” as used herein refers to Cav-1 peptides which have been chemically modified by using techniques including, but not limited to, acetylation, ubiquitination, labeling, pegylation (derivatization with polyethylene glycol), lipidation, glycosylation, amidation, cyclization, or addition of other molecules. In some embodiments, the peptide is provided in a cyclic form, for example, as a cyclic peptide or as a lactam. Alternatively, or in addition, in some embodiments, the peptide is provided as a branched peptide. A molecule is also a “derivative” of another molecule when it contains additional chemical moieties not normally a part of the molecule. Such moieties can alter the pH or improve the molecule's solubility, absorption, biological half-life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, PA (1990), incorporated herein, by reference, in its entirety.

The term “functional” when used in conjunction with “derivative” or “variant” refers to a modified Cav-1 peptide that possesses a biological activity (either functional or structural) that is substantially similar to a biological activity of the entity or molecule it is a functional derivative or functional variant thereof (e.g., the native Cav-1 polypeptide). The term functional derivative is intended to include the fragments, analogues or chemical derivatives of a molecule.

In some embodiments, the modified Cav-1 peptide comprises co-translational and post-translational (e.g., C-terminal peptide cleavage) modifications, such as, for example, disulfide-bond formation, glycosylation, acetylation, phosphorylation, proteolytic cleavage (e.g., cleavage by furins or metalloproteases), and the like to the extent that such modifications do not affect the function of the modified Cav-1 peptide.

In some embodiments, the modified Cav-1 peptide is a “retro-inverso peptide”. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous a-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C, J. Chem. Soc. Perkin Trans. 1:697-701 (1985); and U.S. Pat. No. 6,261,569, which are incorporated herein by reference in their entirety.

### C. Terminal Modifications

In some embodiments, the Cav-1 peptides of the present disclosure are modified (when linear) at its amino terminus or carboxy terminus. Examples of amino terminal modifications include, e.g., N-glycated, N-alkylated, N-acetylated or N-acylated amino acid. A terminal modification can include a pegylation. An example of a carboxy terminal modification is a C-terminal amidated amino acid. In some embodiments, the peptides are cross-linked or have a cross-linking site (for example, the modified Cav-1 peptide has a cysteinyl residue and thus forms cross-linked dimers in vitro or in vivo). In some embodiments, one or more peptidyl bonds are replaced by a non-peptidyl linkage; the N-terminus or the C-terminus is replaced, and individual amino acid moieties are modified through treatment with agents capable of reacting with selected side chains or terminal residues, and so forth. Either the C-terminus or the N-terminus of the amino acid sequences, or both, can be linked to a carboxylic acid functional group or an amine functional group, respectively. In some embodiments, the modified Cav-1 peptide comprises an N-terminal modification. In some embodiments, the modified Cav-1 peptide comprises a C-terminal modification. In some embodiments, the modified Cav-1 peptide comprises an N-terminal and a C-terminal modification.

Non-limiting, illustrative examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include, but are not limited to, acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl, phenyl-CO—, substituted phenyl-CO—, benzyl-CO— and (substituted benzyl)-CO—. Examples of alkoxy carbonyl and aryloxy carbonyl groups include, but are not limited to, CH₃—O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, (substituted phenyl)-O—CO—, benzyl-O—CO—, and (substituted benzyl)-O—CO—. In order to facilitate the N-acylation, one to four glycine residues can be present at the N-terminus of the molecule.

Carboxy terminal modifications include acylation with carboxylic acids: formic acid, acetic acid, propionic acid, fatty acid (myristic, palmitic, stearic), succinic acid, and benzoic acid; carbonylation (such as benzyloxycarbonylation (Cbz)); acetylation; and biotinylation. Amino terminal modifications include, but are not limited to: (i) acylation with carboxylic acids: formic acid, acetic acid, propionic acid, fatty acid (myristic, palmitic, stearic, etc), succinic acid, benzoic acid; (ii) carbonylation (such as benzyloxycarbonylation (Cbz)); (iii) biotinylation; (iv) amidation; (v) attachment of dyes such as fluorescein (FITC, FAM, etc.), 7-hydroxy-4-methylcoumarin-3-acetic acid, 7-hydroxycoumarin-3-acetic acid, 7-methoxycoumarin-3-acetic acid and other coumarins; rhodamines (5-carboxyrhodamine 110 or 6G, 5(6)-TAMRA, ROX); N-[4-(4-dimethylamino)phenylazo]benzoic acid (Dabcyl), 2,4-dinitrobenzene (Dnp), 5-dimethylaminonaphthalene-1-sulfonic acid (Dansyl) and other dyes; and (vi) pegylation.

The carboxyl group at the C-terminus of a peptide can be protected, for example, by a group including, but not limited to, an amide (i.e., the hydroxyl group at the C-terminus is replaced with —NH₂, —NHR2 and —NR2R3) or ester (i.e. the hydroxyl group at the C-terminus is replaced with —OR2). R2 and R3 are optionally independently an aliphatic, substituted aliphatic, benzyl, substituted benzyl, aryl or a substituted aryl group. In addition, taken together with the nitrogen atom, R2 and R3 can optionally form a C4 to C8 heterocyclic ring with from about 0-2 additional heteroatoms such as nitrogen, oxygen or sulfur. Non-limiting examples of heterocyclic rings include, but are not limited to, piperidinyl, pyrrolidinyl, morpholinyl, thiomorpholinyl or piperazinyl. Examples of C-terminal protecting groups include, but are not limited to, —NH₂, —NHCH₃, —N(CH₃)₂, —NH(ethyl), —N(ethyl)₂, —N(methyl)(ethyl), —NH(benzyl), —N(C₁-C₄ alkyl)(benzyl), —NH(phenyl), —N(C₁-C₄ alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

### D. Side Chain Modifications

In some embodiments, the modified Cav-1 peptides of the present disclosure comprise a modified amino acid side chain. Non-limiting examples of modifications include carboxymethylation, acylation, phosphorylation, glycosylation, or fatty acylation. Ether bonds can optionally be used to join the serine or threonine hydroxyl to the hydroxyl of a sugar. Amide bonds can optionally be used to join the glutamate or aspartate carboxyl groups to an amino group on a sugar (Gang and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

As used herein the term “chemical modification”, when referring to a modified Cav-1 peptide of the present disclosure, refers to a peptide wherein at least one of its amino acid residues is modified either by natural processes, such as processing or other post-translational modifications, or by chemical modification techniques which are well known in the art. Examples of the numerous known modifications typically include, but are not limited to: acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

Other types of modifications optionally include the addition of a cycloalkane moiety to a biological molecule, such as a protein, as described in PCT Application No. WO 2006/050262, hereby incorporated by reference in its entirety. These moieties are designed for use with biomolecules and may optionally be used to impart various properties to proteins.

Furthermore, optionally any point on a protein may be modified. For example, pegylation of a glycosylation moiety on a protein may optionally be performed, as described in PCT Application No. WO 2006/050247, hereby incorporated by reference in its entirety. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein through a glycosyl linker.

Covalent modifications of the modified Cav-1 peptides of the present disclosure are included within the scope of this invention. Other types of covalent modifications of the peptides are introduced into the molecule by reacting targeted amino acid residues with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidazoyl) propionic acid, chloroacetyl phosphate, N-alkylmaleimides, 3-nitro-2-pyridyl methyl disulfide, 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide is also useful; the reaction is preferably performed in 0.1M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin.

Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and 3-nitro derivatives, respectively. Tyrosyl residues are iodinated using 125 I or 131 I to prepare labeled peptides for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking to a water-insoluble support matrix or surface for use in the method for purifying anti-CHF antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983]), acetylation of the N-terminal amine, and amidation of any C-terminal carboxyl group.

### E. Capping

In some embodiments, the modified Cav-1 peptide is capped at its N- and/or C-terminus. In some embodiments, the modified Cav-1 peptide is capped at its N- and/or C-terminus with an acyl (abbreviated “Ac”) and/or an amido (abbreviated “Am”) group, respectively, for example acetyl (CH₃CO—) at the N-terminus and amido (—NH₂) at the C-terminus. In some embodiments, the modified Cav-1 peptide is capped at its N-terminus with an acyl group, for example, an acetyl (CH₃CO—) at the N-terminus. In some embodiments, the modified Cav-1 peptide is capped at its C-terminus with an amido group, for example, an amido (—NH₂) at the C-terminus.

In some embodiments, the modified Cav-1 peptide is capped at its N-terminus. A broad range of N-terminal capping functions, preferably in a linkage to the terminal amino group, is contemplated, for example:
formyl;
alkanoyl, having from 1 to 10 carbon atoms, such as acetyl, propionyl, butyryl;
alkenoyl, having from 1 to 10 carbon atoms, such as hex-3-enoyl;
alkynoyl, having from 1 to 10 carbon atoms, such as hex-5-ynoyl;
aroyl, such as benzoyl or 1-naphthoyl;
heteroaroyl, such as 3-pyrroyl or 4-quinolonyl;
alkylsulfonyl, such as methanesulfonyl;
arylsulfonyl, such as benzenesulfonyl or sulfanilyl;
heteroarylsulfonyl, such as pyridine-4-sulfonyl;
substituted alkanoyl, having from 1 to 10 carbon atoms, such as 4-aminobutyryl;
substituted alkenoyl, having from 1 to 10 carbon atoms, such as 6-hydroxy-hex-3-enoyl;
substituted alkynoyl, having from 1 to 10 carbon atoms, such as 3-hydroxy-hex-5-ynoyl;
substituted aroyl, such as 4-chlorobenzoyl or 8-hydroxy-naphth-2-oyl;
substituted heteroaroyl, such as 2,4-dioxo-1,2,3,4-tetrahydro-3-methyl-quinazolin-6-oyl;
substituted alkylsulfonyl, such as 2-aminoethanesulfonyl;
substituted arylsulfonyl, such as 5-dimethylamino-1-naphthalenesulfonyl;
substituted heteroarylsulfonyl, such as 1-methoxy-6-isoquinolinesulfonyl;
carbamoyl or thiocarbamoyl;
substituted carbamoyl (R′—NH—CO) or substituted thiocarbamoyl (R′—NH—CS) wherein R′ is alkyl, alkenyl, alkynyl, aryl, heteroaryl, substituted alkyl, substituted alkenyl, substituted alkynyl, substituted aryl, or substituted heteroaryl;
substituted carbamoyl (R′—NH—CO) and substituted thiocarbamoyl (R′—NH—CS) wherein R′ is alkanoyl, alkenoyl, alkynoyl, aroyl, heteroaroyl, substituted alkanoyl, substituted alkenoyl, substituted alkynoyl, substituted aroyl, or substituted heteroaroyl, all as above defined.

In some embodiments, the modified Cav-1 peptide is capped at its C-terminus. The C-terminal capping function can either be in an amide or ester bond with the terminal carboxyl. Capping functions that provide for an amide bond are designated as NR¹R² wherein R¹ and R² may be independently drawn from the following group: hydrogen;
alkyl, preferably having from 1 to 10 carbon atoms, such as methyl, ethyl, isopropyl;
alkenyl, preferably having from 1 to 10 carbon atoms, such as prop-2-enyl;
alkynyl, preferably having from 1 to 10 carbon atoms, such as prop-2-ynyl;
substituted alkyl having from 1 to 10 carbon atoms, such as hydroxyalkyl, alkoxyalkyl, mercaptoalkyl, alkylthioalkyl, halogenalkyl, cyanoalkyl, aminoalkyl, alkylaminoalkyl, dialkylaminoalkyl, alkynylalkyl, carboxyalkyl, carbamoylalkyl;
substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkenyl, dialkylaminoalkenyl, alkanoylalkenyl, carboxyalkenyl, carbamoylalkenyl;
substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl;
aroylalkyl having up to 10 carbon atoms, such as phenacyl or 2-benzoylethyl;
aryl, such as phenyl or 1-naphthyl;
heteroaryl, such as 4-quinolyl;
alkanoyl having from 1 to 10 carbon atoms, such as acetyl or butyryl;
aroyl, such as benzoyl;
heteroaroyl, such as 3-quinolonyl;
OR′ or NR′R″ where R′ and R″ are independently hydrogen, alkyl, aryl, heteroaryl, acyl, aroyl, sulfonyl, sulfinyl, or SO₂—R′″ or SO—R′″ where R′″ is substituted or unsubstituted alkyl, aryl, heteroaryl, alkenyl, or alkynyl.

Capping functions that provide for an ester bond are designated as OR, wherein R may be: alkoxy; aryloxy; heteroaryloxy; aralkyloxy; heteroaralkyloxy; substituted alkoxy; substituted aryloxy; substituted heteroaryloxy; substituted aralkyloxy; or substituted heteroaralkyloxy.

In some embodiments, the N-terminal or the C-terminal capping function, or both, is of such structure that the capped molecule functions as a prodrug (a pharmacologically inactive derivative of the parent drug molecule) that undergoes spontaneous or enzymatic transformation within the body in order to release the active drug and that has improved delivery properties over the parent drug molecule (Bundgaard H, Ed: Design of Prodrugs, Elsevier, Amsterdam, 1985).

Judicious choice of capping groups allows the addition of other activities on the peptide. For example, the presence of a sulfhydryl group linked to the N- or C-terminal cap will permit conjugation of the derivatized peptide to other molecules.

### F. Multimerization

Embodiments of the present disclosure also include longer polypeptides built from repeating units of a modified Cav-1 peptide. In some embodiments, a polypeptide multimer comprises different combinations of polypeptide. In some embodiments, multimeric polypeptides are made by chemical synthesis or by recombinant DNA techniques as discussed herein. When produced by chemical synthesis, the oligomers, in some embodiments, preferably have from 2-5 repeats of a core polypeptide sequence, and the total number of amino acids in the multimer should not exceed about 160 residues, preferably not more than 100 residues (or their equivalents, when including linkers or spacers).

In some embodiments, the modified Cav-1 peptide is a multimer comprising at least two peptides of the present disclosure. In some embodiments, a first peptide of the at least two peptides is essentially identical to a second peptide of the at least two peptides. In some embodiments, a first peptide of the at least two peptides is not identical to a second peptide of the at least two peptides.

### G. Peptidomimetics

In some embodiments, the modified Cav-1 peptide is a peptidomimetic compound which mimics the biological effects of the native Cav-1 polypeptide. In some embodiments, the peptidomimetic is an unnatural peptide or a non-peptide agent that recreates the stereospatial properties of the binding elements of the native Cav-1 polypeptide such that it has the binding activity and biological activity of the native Cav-1 polypeptide. Similar to a native Cav-1 polypeptide or polypeptide multimer, a peptidomimetic will have a binding face, which interacts with any ligand to which the native Cav-1 polypeptide binds, and a non-binding face.

In some embodiments, the present disclosure also includes modified Cav-1 peptides that retain partial peptide characteristics. For example, any proteolytically unstable bond within the modified Cav-1 peptide could be selectively replaced by a non-peptidic element such as an isostere (N-methylation; D-amino acid) or a reduced peptide bond while the rest of the molecule retains its peptidic nature.

Peptidomimetic compounds, either agonists, substrates or inhibitors, have been described for a number of bioactive peptides/polypeptides such as opioid peptides, VIP, thrombin, HIV protease, etc. Methods for designing and preparing peptidomimetic compounds are known in the art (Hruby, V J, Biopolymers 33:1073-1082 (1993); Wiley, R A et al., Med. Res. Rev. 13:327-384 (1993); Moore et al., Adv. in Pharmacol 33:91-141 (1995); Giannis et al., Adv. in Drug Res. 29:1-78 (1997). Certain mimetics that mimic secondary structure are described in Johnson et al., In: Biotechnology and Pharmacy, Pezzuto et al., Chapman and Hall (Eds.), NY, 1993. These methods are used to make peptidomimetics that possess at least the binding capacity and specificity of the native Cav-1 polypeptide and preferably also possess the biological activity. Knowledge of peptide chemistry and general organic chemistry available to those skilled in the art are sufficient, in view of the present disclosure, for designing and synthesizing such compounds.

For example, such peptidomimetics may be identified by inspection of the three-dimensional structure of a polypeptide of the invention either free or bound in complex with a ligand (e.g., soluble uPAR or a fragment thereof). Alternatively, the structure of a polypeptide of the invention bound to its ligand can be gained by the techniques of nuclear magnetic resonance spectroscopy. Greater knowledge of the stereochemistry of the interaction of the peptide with its ligand or receptor will permit the rational design of such peptidomimetic agents. The structure of a peptide or polypeptide of the invention in the absence of ligand could also provide a scaffold for the design of mimetic molecules.

### H. PEGylation

In some embodiments, the modified Cav-1 peptides of the present disclosure are conjugated with heterologous polypeptide segments or polymers, such as polyethylene glycol. In some embodiments, the modified Cav-1 peptides are linked to PEG to increase the hydrodynamic radius of the enzyme and hence increase the serum persistence. In some embodiments, the modified Cav-1 peptides are conjugated to any targeting agent, such as a ligand having the ability to specifically and stably bind to an external receptor (see e.g., U.S. Patent Publ. No. 2009/0304666).

In some embodiments, the present disclosure provides methods and compositions related to PEGylation of Cav-1 peptides. PEGylation is the process of covalent attachment of poly (ethylene glycol) polymer chains to another molecule, normally a drug or therapeutic protein. PEGylation is routinely achieved by incubation of a reactive derivative of PEG with the target macromolecule. The covalent attachment of PEG to a drug or therapeutic protein can “mask” the agent from the host's immune system (reduced immunogenicity and antigenicity) or increase the hydrodynamic size (size in solution) of the agent, which prolongs its circulatory time by reducing renal clearance. PEGylation can also provide water solubility to hydrophobic drugs and proteins.

The first step of PEGylation is the suitable functionalization of the PEG polymer at one or both terminals. PEGs that are activated at each terminus with the same reactive moiety are known as “homobifunctional,” whereas if the functional groups present are different, then the PEG derivative is referred as “heterobifunctional” or “heterofunctional.” The chemically active or activated derivatives of the PEG polymer are prepared to attach the PEG to the desired molecule.

The choice of the suitable functional group for the PEG derivative is based on the type of available reactive group on the modified Cav-1 peptide that will be coupled to the PEG. For proteins, typical reactive amino acids include lysine, cysteine, histidine, arginine, aspartic acid, glutamic acid, serine, threonine, and tyrosine. The N-terminal amino group and the C-terminal carboxylic acid group can also be used.

The techniques used to form first generation PEG derivatives are generally reacting the PEG polymer with a group that is reactive with hydroxyl groups, typically anhydrides, acid chlorides, chloroformates, and carbonates. In the second generation PEGylation chemistry more efficient functional groups, such as aldehyde, esters, amides, etc., are made available for conjugation.

As applications of PEGylation have become more and more advanced and sophisticated, there has been an increased need for heterobifunctional PEGs for conjugation. These heterobifunctional PEGs are very useful in linking two entities, where a hydrophilic, flexible, and biocompatible spacer is needed. Preferred end groups for heterobifunctional PEGs are maleimide, vinyl sulfones, pyridyl disulfide, amine, carboxylic acids, and N-hydroxysuccinimide (NHS) esters.

The most common modification agents, or linkers, are based on methoxy PEG (mPEG) molecules. Their activity depends on adding a protein-modifying group to the alcohol end. In some embodiments, polyethylene glycol (PEG diol) is used as the precursor molecule. The diol is subsequently modified at both ends in order to make a hetero- or homo-dimeric PEG-linked molecule.

Proteins are generally PEGylated at nucleophilic sites, such as unprotonated thiols (cysteinyl residues) or amino groups. Examples of cysteinyl-specific modification reagents include PEG maleimide, PEG iodoacetate, PEG thiols, and PEG vinylsulfone. All four are strongly cysteinyl-specific under mild conditions and neutral to slightly alkaline pH but each has some drawbacks. The thioether formed with the maleimides can be somewhat unstable under alkaline conditions so there may be some limitation to formulation options with this linker. The carbamothioate linkage formed with iodo PEGs is more stable, but free iodine can modify tyrosine residues under some conditions. PEG thiols form disulfide bonds with protein thiols, but this linkage can also be unstable under alkaline conditions. PEG-vinylsulfone reactivity is relatively slow compared to maleimide and iodo PEG; however, the thioether linkage formed is quite stable. Its slower reaction rate also can make the PEG-vinylsulfone reaction easier to control.

Site-specific PEGylation at native cysteinyl residues is seldom carried out, since these residues are usually in the form of disulfide bonds or are required for biological activity. On the other hand, site-directed mutagenesis can be used to incorporate cysteinyl PEGylation sites for thiol-specific linkers. The cysteine mutation must be designed such that it is accessible to the PEGylation reagent and is still biologically active after PEGylation.

Amine-specific modification agents include PEG NHS ester, PEG tresylate, PEG aldehyde, PEG isothiocyanate, and several others. All react under mild conditions and are very specific for amino groups. The PEG NHS ester is probably one of the more reactive agents; however, its high reactivity can make the PEGylation reaction difficult to control on a large scale. PEG aldehyde forms an imine with the amino group, which is then reduced to a secondary amine with sodium cyanoborohydride. Unlike sodium borohydride, sodium cyanoborohydride will not reduce disulfide bonds. However, this chemical is highly toxic and must be handled cautiously, particularly at lower pH where it becomes volatile.

Due to the multiple lysine residues on most proteins, site-specific PEGylation can be a challenge. Because these reagents react with unprotonated amino groups, it is possible to direct the PEGylation to lower-pK amino groups by performing the reaction at a lower pH. Generally, the pK of the alpha-amino group is 1-2 pH units lower than the epsilon-amino group of lysine residues. By PEGylating the molecule at pH 7 or below, high selectivity for the N-terminus frequently can be attained. However, this is only feasible if the N-terminal portion of the protein is not required for biological activity. Still, the pharmacokinetic benefits from PEGylation frequently outweigh a significant loss of in vitro bioactivity, resulting in a product with much greater in vivo bioactivity regardless of PEGylation chemistry.

There are several parameters to consider when developing a PEGylation procedure. Fortunately, there are usually no more than four or five key parameters. The “design of experiments” approach to optimization of PEGylation conditions can be very useful. For thiol-specific PEGylation reactions, parameters to consider include: protein concentration, PEG-to-protein ratio (on a molar basis), temperature, pH, reaction time, and in some instances, the exclusion of oxygen. (Oxygen can contribute to intermolecular disulfide formation by the protein, which will reduce the yield of the PEGylated product.) The same factors should be considered (with the exception of oxygen) for amine-specific modification except that pH may be even more critical, particularly when targeting the N-terminal amino group.

For both amine- and thiol-specific modifications, the reaction conditions may affect the stability of the protein. This may limit the temperature, protein concentration, and pH. In addition, the reactivity of the PEG linker should be known before starting the PEGylation reaction. For example, if the PEGylation agent is only 70 percent active, the amount of PEG used should ensure that only active PEG molecules are counted in the protein-to-PEG reaction stoichiometry.

### I. Fusion Proteins

In some embodiments, the present disclosure provides fusion proteins of the modified Cav-1 peptides. For example, fusions may employ leader sequences from other species to permit the recombinant expression of a protein in a heterologous host. Fusion proteins can comprise a half-life extender. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST). In some embodiments, the modified Cav-1 peptide comprises a heterologous peptide or protein linked at the N- and/or C-terminus. In some embodiments, the heterologous peptide or protein is a leader sequence, a half-life extender, a protein affinity tag, or an immunologically active domain.

In some embodiments, the modified Cav-1 peptide is linked to a peptide that increases the in vivo half-life, such as an XTEN® polypeptide (Schellenberger et al., 2009), IgG Fc domain, albumin, or albumin-binding peptide.

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

### 1. Linkers

In some embodiments, the modified Cav-1 peptide is chemically conjugated using bifunctional cross-linking reagents or fused at the protein level with peptide linkers.

Bifunctional cross-linking reagents have been extensively used for a variety of purposes, including preparation of affinity matrices, modification and stabilization of diverse structures, identification of ligand and receptor binding sites, and structural studies. In some embodiments, peptide linkers such as Gly-Ser linkers are used to link the modified Cav-1 peptides of the present disclosure.

Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino-, sulfhydryl-, guanidine-, indole-, carboxyl-specific groups. Of these, reagents directed to free amino groups have become especially popular because of their commercial availability, ease of synthesis, and the mild reaction conditions under which they can be applied.

A majority of heterobifunctional cross-linking reagents contain a primary amine-reactive group and a thiol-reactive group. In another example, heterobifunctional cross-linking reagents and methods of using the cross-linking reagents are described (U.S. Pat. No. 5,889,155, incorporated herein by reference in its entirety). The cross-linking reagents combine a nucleophilic hydrazide residue with an electrophilic maleimide residue, allowing coupling, in one example, of aldehydes to free thiols. The cross-linking reagent can be modified to cross-link various functional groups.

Additionally, any other linking/coupling agents and/or mechanisms known to those of skill in the art may be used to combine the modified Cav-1 peptides of the present disclosure, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

In some embodiments, the modified Cav-1 peptide comprises a cross-linker that has reasonable stability in the blood. Numerous types of disulfide-bond containing linkers are known that can be successfully employed to conjugate targeting and therapeutic/preventative agents. Linkers that contain a disulfide bond that is sterically hindered may prove to give greater stability in vivo. Thus, in some embodiments, the modified Cav-1 peptide comprises a sterically hindered cross-linker.

In addition to hindered cross-linkers, non-hindered linkers also can be employed in accordance herewith. In some embodiments, the modified Cav-1 peptide comprises a non-sterically hindered cross linker. Other useful cross-linkers, not considered to contain or generate a protected disulfide, include SATA, SPDP, and 2-iminothiolane (Wawrzynczak and Thorpe, 1987). The use of such cross-linkers is well understood in the art.

In some embodiments, the modified Cav-1 peptide comprises a flexible linker.

Once chemically conjugated, the modified Cav-1 peptide generally will be purified to separate the conjugate from unconjugated agents and from other contaminants. A large number of purification techniques are available for use in providing conjugates of a sufficient degree of purity to render them clinically useful.

Purification methods based upon size separation, such as gel filtration, gel permeation, or high performance liquid chromatography, will generally be of most use. Other chromatographic techniques, such as Blue-Sepharose separation, may also be used. Conventional methods to purify the fusion proteins from inclusion bodies may be useful, such as using weak detergents, such as sodium N-lauroyl-sarcosine (SLS).

### 2. Cell Penetrating and Membrane Translocation Peptides

In some embodiments, the modified Cav-1 peptides comprises a cell-binding domain or cell penetrating peptide (CPP). As used herein, the terms “cell penetrating peptide”, “membrane translocation domain”, and “protein transduction domain” are used interchangeably and refer to segments of a polypeptide sequence that allow a polypeptide to cross the cell membrane (e.g., the plasma membrane in the case a eukaryotic cell). Examples of CPPs include, but are not limited to, segments derived from HIV-binding peptides, HIV-1 Tat (HIV), Tat-derived peptides, Penetratin, VP22 derived or analog peptides, HSV VP22 (Herpes simplex), protegrin I, MAP, KALA or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides (particularly from Drosophila Antennapedia), pAntp, T1 (TKIESLKEHG, SEQ ID NO: 115), T2 (TQIENLKEKG, SEQ ID NO: 116), 26 (AALEALAEALEALAEALEALAEAAAA, SEQ ID NO: 117), INF7 (GLFEAIEGFIENGWEGMIEGWYGCG, SEQ ID NO: 118) pIsl, FGF, Lactoferrin, Transportan, Buforin-2, Bac715-24, SynB, SynB(1), pVEC, hCT-derived peptides, SAP, or histones.

CPPs typically have an amino acid composition that either contains a high relative abundance of positively charged amino acids such as lysine or arginine or have a sequence that contains an alternating pattern of polar/charged amino acids and non-polar, hydrophobic amino acids. These two types of structures are referred to as polycationic or amphipathic, respectively. Typically, CPPs are peptides of 8 to 50 residues that have the ability to cross the cell membrane and enter into most cell types. Frankel and Pabo described the ability of the trans-activating transcriptional activator from the human immunodeficiency virus 1 (HIV-TAT) to penetrate into cells (Frankel, A. D. and C. O. Pabo, Cellular uptake of the tat protein from human immunodeficiency virus. Cell, 1988. 55(6): p. 1189-93). In 1991, transduction into neural cells of the Antennapedia homeodomain (DNA-binding domain) from Drosophila melanogaster was also described (Joliot, A., et al., Antennapedia homeobox peptide regulates neural morphogenesis. Proc Natl Acad Sci USA, 1991. 88(5): p. 1864-8). In 1994, the first 16-mer peptide CPP called Penetratin (RQIKIWFQNRRMKWKK, SEQ ID NO: 113) was characterized from the third helix of the homeodomain of Drosophila Antennapedia homeobox gene product (Derossi, D., et al., The third helix of the Antennapedia homeodomain translocates through biological membranes. J Biol Chem, 1994. 269(14): p. 10444-50), followed in 1998 by the identification of the minimal domain of TAT required for protein transduction (e.g., GRKKRRQRRRPPQ, SEQ ID NO: 112) (Vives, E., P. Brodin, and B. Lebleu, A truncated HIV-1 Tat protein basic domain rapidly translocates through the plasma membrane and accumulates in the cell nucleus. J Biol Chem, 1997. 272(25): p. 16010-7). Over the past two decades, dozens of peptides were described from different origins including viral proteins, e.g., herpes virus VP22 (Elliott, G. and P. O'Hare, Intercellular trafficking and protein delivery by a herpesvirus structural protein. Cell, 1997. 88(2): p. 223-33), or from venoms, e.g. melittin (GIGAVLKVLTTGLPALISWIKRKRQQ, SEQ ID NO: 114) (Dempsey, C. E., The actions of melittin on membranes. Biochim Biophys Acta, 1990. 1031 (2): p. 143-61), mastoporan (Konno, K., et ah, Structure and biological activities of eumenine mastoparan-AF (EMP-AF), a new mast cell degranulating peptide in the venom of the solitary wasp (Anterhynchium flavomarginatum micado). Toxicon, 2000. 38(11): 1505-15), maurocalcin (Esteve, E., et al., Transduction of the scorpion toxin maurocalcine into cells. Evidence that the toxin crosses the plasma membrane. J Biol Chem, 2005. 280(13): p. 12833-9), crotamine (Nascimento, F. D., et al., Crotamine mediates gene delivery into cells through the binding to heparan sulfate proteoglycans. J Biol Chem, 2007. 282(29): p. 21 349-60) or buforin (Kobayashi, S., et al., Membrane translocation mechanism of the antimicrobial peptide buforin 2. Biochemistry, 2004. 43(49): p. 15610-6). Synthetic CPPs were also designed including the poly-arginine (R8, R9, R10 and R12) (Futaki, S., et al., Arginine-rich peptides. An abundant source of membrane-permeable peptides having potential as carriers for intracellular protein delivery. J Biol Chem, 2001. 276(8): p. 5836-40) or transportan (Pooga, M., et al., Cell penetration by transportan. FASEB J, 1998. 12(1): p. 67-77). Any of the above described CPPs may be used in the modified Cav-1 peptides of the present disclosure. A number of other CPPs described in Milletti F. (Drug Discov Today 17 (15-16): 850-60, 2012), can also be used in the modified Cav-1 peptides of the present disclosure.

In some embodiments, the modified Cav-1 peptide comprises an internalization sequence. In some embodiments, the internalization sequence is located at either the C-terminal or N-terminal end of the modified Cav-1 peptide. In some embodiments, the internalization sequence comprises an amino acid sequence selected from the group comprising: GRKKRRQRRRPPQ (SEQ ID NO: 112), RQIKIWFQNRRMKWKK (SEQ ID NO: 113), and GIGAVLKVLTTGLPALISWIKRKRQQ (SEQ ID NO: 114).

### III. Pharmaceutical Compositions

Where clinical applications are contemplated, it may be necessary to prepare modified Cav-1 peptides or pharmaceutical compositions thereof in a form appropriate for the intended application. In some embodiments, the modified Cav-1 peptide or pharmaceutical composition thereof comprises an effective amount of one or more of the modified Cav-1 peptides of the present disclosure dissolved or dispersed in a pharmaceutically acceptable carrier. In some embodiments, the modified Cav-1 peptide or pharmaceutical composition thereof comprises an effective amount of one or more of the modified Cav-1 peptides of the present disclosure and at least one additional therapeutic agent dissolved or dispersed in a pharmaceutically acceptable carrier. The preparation of a pharmaceutical composition that contains at least one modified Cav-1 peptide of the present disclosure and/or at least one additional therapeutic agent will be known to those of skill in the art in light of the present disclosure, as exemplified by Remington's Pharmaceutical Sciences, 18th Ed., 1990, incorporated herein by reference. Moreover, for animal (e.g., human) administration, it will be understood that preparations should meet sterility, pyrogenicity, general safety, and purity standards as required by the FDA Office of Biological Standards.

In some embodiments, the modified Cav-1 peptide or pharmaceutical composition thereof comprises different types of carriers depending on whether the composition is to be administered in solid, liquid, or aerosol form, and whether it needs to be sterile for the route of administration, such as injection.

In some embodiments, the modified Cav-1 peptides or pharmaceutical compositions thereof are formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups can also be derived from inorganic bases, such as, for example, sodium, potassium, ammonium, calcium, or ferric hydroxides; or such organic bases as isopropylamine, trimethylamine, histidine, or procaine. Upon formulation, solutions will be administered in a manner compatible with the dosage formulation and in such amount as is therapeutically effective. The formulations are easily administered in a variety of dosage forms, such as formulated for parenteral administrations, such as injectable solutions, or aerosols for delivery to the lungs, or formulated for alimentary administrations, such as drug release capsules and the like.

In some embodiments, the modified Cav-1 peptide or pharmaceutical composition thereof is provided in a pharmaceutically acceptable carrier with or without an inert diluent. The carrier should be assimilable and includes liquid, semi-solid, i.e., pastes, or solid carriers. Except insofar as any conventional media, agent, diluent, or carrier is detrimental to the recipient or to the therapeutic effectiveness of a composition contained therein, its use in administrable composition for use in practicing the methods is appropriate. Examples of carriers or diluents include fats, oils, water, saline solutions, lipids, liposomes, resins, binders, fillers, and the like, or combinations thereof.

In some embodiments, the modified Cav-1 peptide or pharmaceutical composition thereof is combined with the carrier in any convenient and practical manner, i.e., by solution, suspension, emulsification, admixture, encapsulation, absorption, and the like. Such procedures are routine for those skilled in the art. In some embodiments, the modified Cav-1 peptide or pharmaceutical composition thereof is combined or mixed thoroughly with a semi-solid or solid carrier. The mixing can be carried out in any convenient manner, such as grinding.

In some embodiments, the modified Cav-1 peptide or pharmaceutical composition thereof comprises one or more antioxidants to retard oxidation of one or more components in the composition. Additionally, the prevention of the action of microorganisms can be brought about by preservatives, such as various antibacterial and antifungal agents, including but not limited to parabens (e.g., methylparabens, propylparabens), chlorobutanol, phenol, sorbic acid, thimerosal or combinations thereof.

In some embodiments, the modified Cav-1 peptide or pharmaceutical composition thereof comprises one or more stabilizing agents. Stabilizing agents can be also added in the mixing process in order to protect the composition from loss of therapeutic activity, e.g., denaturation in the stomach. Examples of stabilizers include, but are not limited to, buffers, amino acids, such as glycine and lysine, carbohydrates or lyoprotectants, such as dextrose, mannose, galactose, fructose, lactose, sucrose, maltose, sorbitol, mannitol, etc.

In some embodiments, the modified Cav-1 peptide or pharmaceutical composition thereof comprises one or more surfactants. Surfactants used in accordance with the disclosed methods include ionic and non-ionic surfactants. Representative non-ionic surfactants include polysorbates such as TWEEN®-20 and TWEEN-80® surfactants (ICI Americas Inc. of Bridgewater, N.J.); poloxamers (e.g., poloxamer 188); anionic and nonionic surfactants such as TRITON® surfactants (Sigma of St. Louis, Mo.); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; cationic or quaternary phospholipid surfactants such as MONAQUAT™ surfactants (Mona Industries Inc. of Paterson, N.J.); polyethyl glycol; polypropyl glycol; block copolymers of ethylene and propylene glycol such as PLURONIC® surfactants (BASF of Mt. Olive, N.J.); oligo (ethylene oxide) alkyl ethers; alkyl (thio) glucosides, alkyl maltosides; and phospholipids. In some embodiments, the one or more surfactants are present in the modified Cav-1 peptide or pharmaceutical composition thereof in an amount from about 0.01% to about 0.5% (weight of surfactant relative to total weight of other solid components of the formulation; “w/w”), from about 0.03% to about 0.5% (w/w), from about 0.05% to about 0.5% (w/w), or from about 0.1% to about 0.5% (w/w). In some embodiments, the modified Cav-1 peptide or pharmaceutical composition thereof is essentially free of non-ionic surfactants or essentially free of all surfactants.

In some embodiments, the modified Cav-1 peptide or pharmaceutical composition thereof is formulated as a dry powder composition. In order to effectively inhale and deposit a powder into the lungs, the particle size should generally have a mass median aerodynamic diameter of less than about 5 μm. In some embodiments, the dry powder formulation comprising the modified Cav-1 peptide comprises an average particle size of less than 10 μm. In some embodiments, the dry powder formulation comprising the modified Cav-1 peptide comprises an average particle size of less than 5 μm. In some embodiments, the dry powder formulation comprising the modified Cav-1 peptide comprises an average particle size of less than 1 μm. In some embodiments, the modified Cav-1 peptide comprises an average particle size of about 0.01 μm to about 10 μm, about 0.1 μm to about 8 μm, about 0.5 μm to about 7 μm, or about 1 μm to about 5 μm. In some embodiments, the dry powder formulation comprising the modified Cav-1 peptide comprises an average particle size of about 0.1 μm, about 0.5 μm, about 1 μm, about 2 μm, about 3 μm, about 4 μm, about 5 μm, about 6 μm, about 7 μm, about 8 μm, about 9 μm, about 10 μm, or any range or value therebetween. In some embodiments, 90% of the dry powder has a particle size below about 10 μm (Dv90 of about 10 μm). In some embodiments, Dv90 of the dry powder is about 5 μm. The particle sizes of the modified Cav-1 peptides or pharmaceutical compositions thereof can be reduced by any suitable method, including but not limited to milling, grinding, thin film freezing, spray drying, or crushing. Milling may be performed by any method known in the art, such as by air jet mill, ball mill, wet mill, media mill, high pressure homogenization, or cryogenic mill. See WO2020/055824, which is incorporated by reference herein in its entirety. In some embodiments, the dry powder of modified Cav-1 peptide is substantially excipient free. In some embodiments, the dry powder of modified Cav-1 peptide is excipient free. In some embodiments, the dry powder consists of the Cav-1 peptide.

Peptide stability following particle size reduction can be assessed using known techniques in the art, including size exclusion chromatography; electrophoretic techniques; HPLC; mass spectrometry; spectroscopic techniques such as UV spectroscopy and circular dichroism spectroscopy, and activity (measured in vitro or in vivo). To perform in vitro assays of protein stability, an aerosol composition can be collected and then distilled or absorbed onto a filter. To perform in vivo assays, or for pulmonary administration of a composition to a subject, a device for dry powder dispersion is adapted for inhalation by the subject. For example, protein stability can be assessed by determining the level of protein aggregation. In some embodiments, a dry powder composition of the modified Cav-1 peptide is substantially free of protein aggregates. The presence of soluble aggregates can be determined qualitatively using dynamic light scattering (DLS) (DynaPro-801TC, Protein Solutions Inc. of Charlottesville, Va.) and/or by UV spectrophotometry.

In some embodiments, treatment of a subject with milled modified Cav-1 peptide comprise modulated drug release. In some embodiments, milled modified Cav-1 peptide is formulated for slow- or delayed-release. In some embodiments, milled modified Cav-1 peptide is formulated for fast-release. In further embodiments, milled modified Cav-1 peptide is formulated for both slow and fast release (i.e., dual release profile).

In some embodiments, the present disclosure provides methods for the administration of the inhalable modified Cav-1 peptides disclosed herein. Administration via inhalation includes, but is not limited to, use of an inhaler or nebulizer.

In some embodiments, an inhaler is a passive dry powder inhaler (DPI), such as a Plastiape RS01 monodose DPI. In a dry powder inhaler, dry powder is stored in a reservoir and is delivered to the lungs by inhalation without the use of propellants. In some embodiments, an inhaler is a single-dose DPI, such as a DoseOne™, Spinhaler®, Rotohaler®, Aerolizer®, or Handihaler®. In some embodiments, an inhaler is a multidose DPI, such as a Plastiape RS02, Turbuhaler®, Twisthaler™, Diskhaler®, Diskus®, or Ellipta™. In some embodiments, an inhaler is a plurimonodose DPI for the concurrent delivery of single doses of multiple medications, such as a Plastiape RS04 plurimonodose DPI. Typically, dry powder inhalers have medication stored in an internal reservoir, and medication is delivered by inhalation with or without the use of propellants. Other types of dry powder inhalers have medication in pre-divided doses stored in a capsule (e.g., cellulose or gelatin base) or foil pouch, each of which is punctured by the device to release the dose to the patient. Dry powder inhalers may require an inspiratory flow rate greater than about 30 L/min for effective delivery, such as between about 30 L/min to about 120 L/min. In some embodiments, efficient aerosolization of milled modified Cav-1 peptide is independent of inspiratory force. In some embodiments, the dry powder inhaler has a flow resistance of between about 0.01 kPa^{0.5} min/L and about 0.05 kPa^{0.5} min/L, such as between about 0.02 kPa^{0.5} min/L and about 0.04 kPa^{0.5} min/L. The dry powder inhaler (e.g., high resistance, low resistance, passive, active) is chosen based on the patient population and their inspiratory capabilities.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator, and may utilize a spacer device to de-accelerate the emitted particles and facilitate inhalation of the aerosolized cloud by the patient. The medication formulation, including propellants and any required excipients, are stored in the canister. The metering valve allows a defined quantity of the medication formulation to be dispensed. The actuator of the metered dose inhaler, or mouthpiece, contains the mating discharge nozzle and typically includes a dust cap to prevent contamination. In some embodiments, the required inspiratory flow rate required for the use of a metered dose inhaler is less than about 90 L/min, such as between about 15 L/min and about 90 L/min, preferably about 30 L/min. In some embodiments, efficient aerosolization of milled modified Cav-1 peptide is independent of inspiratory force.

In some embodiments, the modified Cav-1 peptide or pharmaceutical composition thereof is delivered by inhalation to a subject through the use of a nebulizer. A nebulizer is used to deliver medication in the form of an aerosolized mist inhaled into the lungs. The medication formulation is aerosolized by compressed gas, or by ultrasonic waves. A jet nebulizer is connected to a compressor. The compressor emits compressed gas through a liquid medication formulation at a high velocity, causing the medication formulation to aerosolize. Aerosolized medication is then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication is then inhaled by the patient. A nebulizer may utilize a flow rate of between about 3 L/min and about 12 L/min, such as about 6 L/min. In some examples, the milled modified Cav-1 peptide can be suspended in a pharmaceutically acceptable liquid carrier vehicle and administered by nebulization (e.g., air jet nebulization). In some embodiments, the modified Cav-1 peptides are administered by a vaporization method (e.g., rapid vaporization) such as by an e-cigarette device.

In some embodiments, the modified Cav-1 peptide or pharmaceutical composition thereof is nebulized into aerosol droplets. In some embodiments, the aerosol droplets have a median diameter of about 1 μm to about 20 μm. In some embodiments, the aerosol droplets have a median diameter of about 2.5 μm to about 20 μm. In some embodiments, the aerosol droplets have a median diameter of about 2 μm to about 10 μm. In some embodiments, the aerosol droplets have a median diameter of about 2 μm to about 4 μm. In some embodiments, the aerosol droplets have a median diameter of about 1 μm to about 5 μm. In some embodiments, the aerosol droplets have a median diameter of about 0.5 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, or any size in-between.

### IV. Methods of Use

The present disclosure provides use of modified Cav-1 peptides or pharmaceutical compositions thereof described herein. Specifically, these methods relate to administering any one of the modified Cav-1 peptides described herein or pharmaceutical compositions thereof to a subject. In some embodiments, the modified Cav-1 peptides or pharmaceutical compositions thereof are used in the treatment or prevention of a disease or disorder of the kidney (e.g., chronic kidney disease) in a subject. In some embodiments, the modified Cav-1 peptides or pharmaceutical compositions thereof are used in the treatment or prevention of a disease or disorder of the lungs (e.g., idiopathic pulmonary fibrosis) in a subject. In some embodiments, the subject is elderly or of advanced age.

In some embodiments, the modified Cav-1 peptides or pharmaceutical compositions thereof are used in the treatment or prevention of a disease or disorder of the kidney (e.g., chronic kidney disease) in a subject. In some embodiments, the modified Cav-1 peptide is used to treat or prevent a kidney disease or disorder such as, for example, chronic kidney disease, end-stage renal disease, glomerulonephritis, focal segmental glomerulosclerosis, kidney fibrosis, polycystic kidney disease, IgA nephropathy, lupus nephritis, nephrotic syndrome, Alport syndrome, amyloidosis, Goodpasture syndrome, Wegener's granulomatosis, or acute kidney injury. In some embodiments, the kidney disease is characterized by fibrosis. In some embodiments, the kidney disease or disorder is acute. In some embodiments, the kidney disease or disorder is chronic. In some embodiments, the subject is elderly or of advanced age. In some embodiments, the kidney disease or disorder in the subject is caused by an infection, such as, for example, a viral, bacterial, fungal, or parasitic infection. In some embodiments, the kidney disease or disorder in the subject is caused by high blood pressure (hypertension). In some embodiments, the kidney disease or disorder in the subject is caused by diabetes. In some embodiments, the kidney disease or disorder in the subject is caused by overuse of drugs, such as over-the counter pain killers and heroine. In some embodiments, the subject has hypertension or diabetes.

In some embodiments, the modified Cav-1 peptides or pharmaceutical compositions thereof are used in delaying the progression of a disease or disorder of the kidney in a subject. In some embodiments, the modified Cav-1 peptides or pharmaceutical compositions thereof are used in improving progression free survival. In some embodiments, the modified Cav-1 peptides or pharmaceutical compositions thereof are used to prolong survival of the subject.

In some embodiments, the modified Cav-1 peptides of the present disclosure are used to treat or prevent chronic kidney disease in a subject. Chronic kidney disease is a gradual and progressive loss of the ability of the kidneys to excrete wastes, concentrate urine, and conserve electrolytes. The progressive loss of renal function occurs as a consequence of the deposition of fibrous tissue between the functional units of the kidney or nephrons (interstitial fibrosis) as well as the ongoing replacement of the filtration surface by fibrous tissue (glomerular sclerosis). Kidney fibrosis is a pathological hallmark of chronic kidney disease and a major contributing factor of progression to end-stage renal disease. In one embodiment, the chronic kidney disease is chronic kidney fibrosis.

In some embodiments, the modified Cav-1 peptides of the present disclosure are used to treat or prevent end-stage renal disease in a subject. End-stage renal disease is the final stage of chronic kidney disease where the kidneys have ceased functioning and the individual requires long-term dialysis or a kidney transplant to survive.

In some embodiments, the modified Cav-1 peptides of the present disclosure are used to treat or prevent focal segmental glomerulosclerosis in a subject. Focal segmental glomerulosclerosis is a kidney disease characterized by scarring of the glomerulus causing loss of protein into the urine.

In some embodiments, the modified Cav-1 peptides of the present disclosure are used to treat or prevent glomerulonephritis in a subject. Glomerulonephritis, also referred to as glomerular disease, is a type of kidney disease in which the glomeruli are damaged and cannot remove waste and fluid properly from the body. In some embodiments, the glomerulonephritis is acute glomerulonephritis. In some embodiments, the glomerulonephritis is chronic glomerulonephritis.

In some embodiments, the modified Cav-1 peptides of the present disclosure are used to treat or prevent polycystic kidney disease in a subject. Polycystic kidney disease is an inherited disorder in which clusters of cysts develop primarily within the kidneys causing the kidneys to enlarge and lose function overtime.

In some embodiments, the modified Cav-1 peptides of the present disclosure are used to treat or prevent IgA nephropathy in a subject. IgA nephropathy, also referred to as Berger's disease, is a kidney disease that occurs when the immunoglobulin IgA accumulates in the kidneys resulting in local inflammation that can prevent the ability of the kidneys to filter waste from the blood.

In some embodiments, the modified Cav-1 peptides of the present disclosure are used to treat or prevent lupus nephritis in a subject. Lupus nephritis is a type of glomerulonephritis that constitutes one of the most severe organ manifestations of the systemic lupus erythematosus and occurs when the immune system attacks the kidneys.

In some embodiments, the modified Cav-1 peptides of the present disclosure are used to treat or prevent nephrotic syndrome in a subject. Nephrotic syndrome is a kidney disorder that causes the body to excrete too much protein into the urine. Nephrotic syndrome is often caused by damage to small blood vessels in the kidneys that filter waste and excess water from the blood.

In some embodiments, the modified Cav-1 peptides of the present disclosure are used to treat or prevent Alport syndrome in a subject. Alport syndrome is a genetic condition characterized by progressive kidney disease and abnormalities of the inner ear and the eye. There are three genetic types: X-linked Alport syndrome (XLAS), autosomal recessive Alport syndrome (ARAS), and autosomal dominant Alport syndrome (ADAS). XLAS is caused by variants in the COL4A5 gene, ARAS is caused by variants in both copies of either the COL4A3 or the COL4A4 gene, and ADAS is caused by variants in one copy of the COL4A3 or COL4A4 gene. Individuals with Alport syndrome present with chronic glomerular dysfunction, renal inflammation, and fibrosis, which are the hallmarks of chronic kidney disease, and progress to end-stage kidney disease. Those afflicted with Alport syndrome can also develop progressive hearing loss of varying severity and abnormalities of the eyes that usually do not result in impaired vision.

In some embodiments, the modified Cav-1 peptides of the present disclosure are used to treat or prevent amyloidosis in a subject. Amyloidosis occurs when amyloid builds up in tissues and organs and interferes with normal function. Amyloid deposits damage the kidney affecting the ability of the kidney to filter wastes and break down proteins. In some embodiments, the amyloidosis is primary amyloidosis. In some embodiments, the amyloidosis is dialysis-related amyloidosis.

In some embodiments, the modified Cav-1 peptides of the present disclosure are used to treat or prevent Goodpasture syndrome in a subject. Goodpasture syndrome, also referred to as anti-glomerular basement membrane disease, is an autoimmune disease in which antibodies attack the basement membrane of the lungs and kidneys leading to pulmonary hemorrhage, glomerulonephritis, and kidney failure.

In some embodiments, the modified Cav-1 peptides of the present disclosure are used to treat or prevent granulomatosis with polyangiitis in a subject. Granulomatosis with polyangiitis, also referred to as Wegener's granulomatosis, is an autoimmune disease involving granulomatous inflammation, necrosis, and vasculitis that most frequently targets the lungs and kidneys.

In some embodiments, the modified Cav-1 peptides of the present disclosure are used to treat or prevent acute kidney injury in a subject. Acute kidney injury, also referred to as acute renal failure, is a sudden loss of excretory kidney function. Acute kidney injury is defined by serum creatine and urine output levels with a duration of less than one week.

In some embodiments, the modified Cav-1 peptide is used to treat or prevent a kidney infection. In some embodiments, the modified Cav-1 peptide is used to treat or prevent pyelonephritis. Pyelonephritis is a type of urinary tract infection where one or both kidneys become infected. In some embodiments, the pyelonephritis is caused by a bacteria or virus, such as, for example, Escherichia coli, Klebsiella, Proteus, Pseudomonas, Enterococcus, or Staphylococcus saprophyticus.

In some embodiments, the modified Cav-1 peptide is used to treat or prevent a kidney disease or disorder resulting from microbial infection in a subject. In some embodiments, the microbial infection is a bacterial, viral, fungal, or parasitic infection. In some embodiments, the kidney disease or disorder is caused by Streptococcus pyogenes, Staphylococcus (aureus, epidermidis), Salmonella (typhi, paratyphi), Escherichia coli, Leptospira, Mycobacterium tuberculosis, Mycobacterium leprae, Legionella spp., Yersinia enterocolitica, Brucella species, Campylobacter jejuni, Corynebacterium diphtheriae, Klebsiella, Proteus, Pseudomonas, Enterococcus, Staphylococcus saprophyticus, SARS-CoV-1, SARS-CoV-2, dengue virus, hantavirus, Varicella-zoster virus, parvovirus, hepatitis A virus, hepatitis B virus, hepatitis E virus, cytomegalovirus, Epstein-Barr virus, human immunodeficiency virus, and/or hepatitis C virus.

In some embodiments, the modified Cav-1 peptide is used to treat or prevent a kidney disease or disorder resulting from infection with SARS-CoV-2. In some embodiments, SARS-CoV-2 causes acute kidney injury. In some embodiments, SARS-CoV-2 causes chronic kidney injury. In some embodiments, SARS-CoV-2 causes chronic kidney disease. In some embodiments, the SARS-CoV-2 causes kidney fibrosis. In some embodiments, the SARS-CoV-2 causes kidney failure.

In some embodiments, the present disclosure provides a method of treating or preventing a kidney disease or disorder in a subject, wherein the method comprises administering to the subject an effective amount of a modified Cav-1 peptide or pharmaceutical composition thereof. In some embodiments, the present disclosure provides a method of treating or preventing a kidney disease or disorder in a subject, wherein the method comprises administering to the subject an effective amount of a modified Cav-1 peptide comprising an amino acid sequence of any one of SEQ ID NOs: 2-111. In some embodiments, the present disclosure provides a method of treating or preventing a kidney disease or disorder in a subject, wherein the method comprises administering to the subject an effective amount of a modified Cav-1 peptide comprising an amino acid sequence of any one of SEQ ID NOs: 4-20. In some embodiments, the present disclosure provides a method of treating or preventing a kidney disease or disorder in a subject, wherein the method comprises administering to the subject an effective amount of a modified Cav-1 peptide comprising an amino acid sequence of SEQ ID NO: 3. In some embodiments, the present disclosure provides a method of treating or preventing a kidney disease or disorder in a subject, wherein the method comprises administering to the subject an effective amount of a modified Cav-1 peptide comprising an amino acid sequence of SEQ ID NO: 8. In some embodiments, the present disclosure provides a method of treating or preventing a kidney disease or disorder in a subject, wherein the method comprises administering to the subject an effective amount of a modified Cav-1 peptide comprising at least one amino acid substitution, deletion of insertion relative to the amino acid sequence of FTTFTVT (SEQ ID NO: 3), wherein the modified Cav-1 peptide maintains the biological activity of Cav-1. In some embodiments, the kidney disease or disorder is chronic kidney disease. In some embodiments, the kidney disease or disorder is Alport syndrome.

In some embodiments, the modified Cav-1 peptides or pharmaceutical compositions thereof are used to improve kidney function in a subject with a kidney disease or disorder. In some embodiments, the modified Cav-1 peptide or pharmaceutical composition thereof improves kidney function by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical composition thereof. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8. In some embodiments, an improvement in kidney function signifies a decrease in fibrotic glomeruli, a decrease in blood urea nitrogen, a decrease in blood creatinine, an increase in blood albumin, a decrease in urine albumin to creatinine ratio, and/or an increase in glomerular filtration rate.

In some embodiments, the modified Cav-1 peptides or pharmaceutical compositions thereof decrease the number of fibrotic glomeruli in a subject with a kidney disease or disorder. In some embodiments, the modified Cav-1 peptide or pharmaceutical composition thereof decreases the number of fibrotic glomeruli by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical composition thereof. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the subject with a kidney disease or disorder has reduced caveolin-1 expression in the kidney compared to a subject without a kidney disease or disorder (e.g., a normal, healthy subject). In some embodiments, caveolin-1 expression is reduced in the glomeruli of the subject with a kidney disease or disorder compared to the subject without a kidney disease or disorder. In some embodiments, caveolin-1 expression is reduced in endothelial cells of the kidney in the subject with a kidney disease or disorder compared to the subject without a kidney disease or disorder. In some embodiments, caveolin-1 expression is reduced in epithelial cells of the kidney in the subject with a kidney disease or disorder compared to the subject without a kidney disease or disorder. In some embodiments, caveolin-1 expression is reduced in podocytes of the kidney in the subject with a kidney disease or disorder compared to the subject without a kidney disease or disorder. In some embodiments, caveolin-1 expression in the kidney is reduced in the subject with a kidney disease or disorder by at least about 10%, at least about 20%, at least about 30%, at least about 40%, at least about 50%, at least about 60%, at least about 70%, at least about 80%, or at least about 90% compared to the subject without a kidney disease or disorder. In some embodiments, the epithelial cells are parietal epithelial cells lining Bowman's capsule.

In some embodiments, the modified Cav-1 peptides or pharmaceutical compositions thereof decreases endothelial cell death in a subject with a kidney disease or disorder. In some embodiments, the modified Cav-1 peptides or pharmaceutical compositions thereof decreases epithelial cell death in a subject with a kidney disease or disorder. In some embodiments, the modified Cav-1 peptides or pharmaceutical compositions thereof decreases podocyte cell death in a subject with a kidney disease or disorder. In some embodiments, the modified Cav-1 peptide or pharmaceutical composition thereof decreases cell death by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical composition thereof. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof increases endothelial cell survival in a subject with a kidney disease or disorder. In some embodiments, the modified Cav-1 peptides or pharmaceutical compositions thereof increases epithelial cell survival in a subject with a kidney disease or disorder. In some embodiments, the modified Cav-1 peptides or pharmaceutical compositions thereof increases podocyte survival in a subject with a kidney disease or disorder. In some embodiments, the modified Cav-1 peptide or pharmaceutical composition thereof increases cell survival by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical composition thereof. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof promotes kidney regeneration in a subject with a kidney disease or disorder. In some embodiments, the modified Cav-1 peptides or pharmaceutical compositions thereof promote regeneration of renal vessels, glomeruli, and/or tubules in the kidney of the subject. In some embodiments, the modified Cav-1 peptides or pharmaceutical compositions thereof promotes regeneration of epithelial cells, endothelial cells, tubular cells, and/or podocytes in the kidney of the subject. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof increases endothelial cell proliferation in a subject with a kidney disease or disorder. In some embodiments, the modified Cav-1 peptides or pharmaceutical compositions thereof increases epithelial cell proliferation in a subject with a kidney disease or disorder. In some embodiments, the modified Cav-1 peptides or pharmaceutical compositions thereof increases podocyte proliferation in a subject with a kidney disease or disorder. In some embodiments, the modified Cav-1 peptide or pharmaceutical composition thereof increases cell proliferation by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical composition thereof. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof decrease blood urea nitrogen in a subject with a kidney disease or disorder. In some embodiments, a high blood urea nitrogen value indicates kidney injury or disease in a subject. In general, a blood urea nitrogen level ranging from 6 mg/dl to 24 mg/dl is considered normal in humans. In some embodiments, the modified Cav-1 peptide or pharmaceutical composition thereof decreases blood urea nitrogen by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical composition thereof. In some embodiments, the modified Cav-1 peptide or pharmaceutical composition thereof decreases blood urea nitrogen in a subject to less than about 50 mg/dl, less than about 45 mg/dl, less than about 40 mg/dl, less than about 35 mg/dl, less than about 30 mg/dl, less than about 25 mg/dl, or less than about 20 mg/dl. In some embodiments, the modified Cav-1 peptide or pharmaceutical composition thereof decreases blood urea nitrogen to less than about 20 mg/dl. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof decrease blood creatinine in a subject with a kidney disease or disorder. In some embodiments, a high blood creatinine value indicates kidney injury or disease in a subject. In general, a blood creatinine value of greater than 1.2 mg/dl in women and 1.4 mg/dl in men signifies that the kidneys are not functioning properly. In some embodiments, the modified Cav-1 peptide or pharmaceutical composition thereof decreases blood creatinine by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical composition thereof. In some embodiments, the modified Cav-1 peptide or pharmaceutical composition thereof decreases blood creatinine in a subject to less than about 4 mg/dl, less than about 3.5 mg/dl, less than about 3.25 mg/dl, less than about 3 mg/dl, less than about 2.75 mg/dl, less than about 2.5 mg/dl, less than about 2.25 mg/dl, less than about 2 mg/dl, less than about 1.75 mg/dl, less than about 1.5 mg/dl, or less than about 1.25 mg/dl. In some embodiments, the modified Cav-1 peptide or pharmaceutical composition thereof decreases blood urea nitrogen to less than about 1.5 mg/dl. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof increase blood albumin in a subject with a kidney disease or disorder. In some embodiments, a low blood albumin value may indicate kidney injury or disease in a subject. The normal level of albumin in the blood is 3.5 g/dL to 5 g/dL. In some embodiments, the modified Cav-1 peptide or pharmaceutical composition thereof increases blood albumin by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical composition thereof. In some embodiments, the modified Cav-1 peptide or pharmaceutical composition thereof increases blood albumin in a subject to greater than about 1.5 g/dl, greater than about 1.75 g/dl, greater than about 2 g/dl, greater than about 2.25 g/dl, greater than about 2.5 g/dl, greater than about 2.75 g/dl, greater than about 3 g/dl, or greater than about 3.5 g/dl. In some embodiments, the modified Cav-1 peptide or pharmaceutical composition thereof increases blood albumin to greater than about 3.5 g/dl. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof decrease the urine albumin to creatinine ratio in a subject with a kidney disease or disorder. The urine albumin to creatinine ratio helps to identify kidney injury or disease in a subject. A ratio of albumin to creatinine of less than 30 mg/g is considered normal; a ratio of 30-300 mg/g signifies microalbuminuria, and values above 300 mg/g signify macroalbuminuria in humans. In some embodiments, the modified Cav-1 peptide or pharmaceutical composition thereof decreases the urine albumin to creatinine ratio by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical composition thereof. In some embodiments, the modified Cav-1 peptide or pharmaceutical composition thereof decreases the urine albumin to creatinine ratio in a subject to less than about 300 mg/g, less than about 250 mg/g, less than about 200 mg/g, less than about 150 mg/g, less than about 100 mg/g, less than about 75 mg/g, less than about 50 mg/g, less than about 40 mg/g, less than about 30 mg/g, or less than about 25 mg/dl. In some embodiments, the modified Cav-1 peptide or pharmaceutical composition thereof decreases the urine albumin to creatinine ratio to less than about 30 mg/dl. In some embodiments, the modified Cav-1 peptide or pharmaceutical composition thereof comprises an amino acid sequence of any one of SEQ ID NOS: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof increase the glomerular filtration rate in a subject with a kidney disease or disorder. The glomerular filtration rate measures how well the kidneys are filtering the blood to remove waste and extra water to make urine. A glomerular filtration rate above 90 mL/min/1.73 m² is considered normal, while a glomerular filtration rate of less than 60 mL/min/1.73 m² may signify kidney injury or disease. A glomerular filtration rate below 15 mL/min/1.73 m² may signify kidney failure. In some embodiments, the modified Cav-1 peptide or pharmaceutical composition thereof increases the glomerular filtration rate by at least about 10%, at least about 20%, at least about 30%, at least about 40%, at least about 50%, at least about 60%, at least about 70%, at least about 80%, at least about 90%, or at least about 100% compared to the subject before treatment with the modified Cav-1 peptide or pharmaceutical composition thereof. In some embodiments, the modified Cav-1 peptide or pharmaceutical composition thereof increases the glomerular filtration rate in a subject to greater than about 30 mL/min/1.73 m², greater than about 40 mL/min/1.73 m², greater than about 50 mL/min/1.73 m², greater than about 60 mL/min/1.73 m², greater than about 70 mL/min/1.73 m², greater than about 80 mL/min/1.73 m², or greater than about 90 mL/min/1.73 m². In some embodiments, the modified Cav-1 peptide or pharmaceutical composition thereof increases the glomerular filtration rate in a subject to greater than about 60 mL/min/1.73 m². In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof are used to preserve kidney function in a subject with a kidney disease or disorder. The term “preserve” as used herein refers to maintaining kidney function or preventing a further decline in kidney function in a subject with a kidney disease or disorder. In some embodiments, the modified Cav-1 peptide or pharmaceutical composition thereof preserves kidney function as measured by blood urine nitrogen, blood creatinine, blood albumin, urine albumin to creatinine ratio, and/or glomerular filtration rate, wherein these measurements remain stable upon treatment with the modified Cav-1 peptide or pharmaceutical composition thereof. In some embodiments, the modified Cav-1 peptide or pharmaceutical composition thereof comprises an amino acid sequence of any one of SEQ ID NOs: 2-111. In some embodiments, the modified Cav-1 peptide or pharmaceutical composition thereof comprises an amino acid sequence of SEQ ID NO: 3. In some embodiments, the modified Cav-1 peptide or pharmaceutical composition thereof comprises an amino acid sequence of SEQ ID NO: 8.

In some embodiments, the modified Cav-1 peptides or pharmaceutical compositions thereof are used in the treatment or prevention of a disease or disorder in an elderly subject. As used herein, the terms “elderly” or “advanced age” refers to a subject 55 years of age or older. In some embodiments, the elderly subject is about 55 years old, about 60 years old, about 65 years old, about 70 years old, about 75 years old, about 80 years old, about 90 years old, about 95 years old, or about 100 years old. In some embodiments, the elderly subject has an increased susceptibility to a disease or disorder described herein compared to a subject of a younger age. In some embodiments, the elderly subject has a fibrotic disease or disorder, e.g., idiopathic pulmonary fibrosis.

In some embodiments, the modified Cav-1 peptide or pharmaceutical composition thereof is used to treat or prevent a fibrotic disease or disorder in an elderly subject. In some embodiments, the modified Cav-1 peptide is used to treat or prevent a fibrotic disease or disorder such as, for example, interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.

In some embodiments, the elderly subject has reduced caveolin-1 expression compared to a younger subject (e.g., a young adult or middle-age subject). In some embodiments, caveolin-1 expression is reduced in the elderly subject by at least about 10%, at least about 20%, at least about 30%, at least about 40%, at least about 50%, at least about 60%, at least about 70%, at least about 80%, or at least about 90% compared to the younger subject.

In some embodiments, the present disclosure provides a method of treating or preventing a fibrotic disease or disorder in an elderly subject, wherein the method comprises administering to the elderly subject an effective amount of a modified Cav-1 peptide or pharmaceutical composition thereof. In some embodiments, the present disclosure provides a method of treating or preventing a fibrotic disease or disorder in an elderly subject, wherein the method comprises administering to the elderly subject an effective amount of a modified Cav-1 peptide comprising an amino acid sequence of any one of SEQ ID NOs: 2-111. In some embodiments, the present disclosure provides a method of treating or preventing a fibrotic disease or disorder in an elderly subject, wherein the method comprises administering to the elderly subject an effective amount of a modified Cav-1 peptide comprising an amino acid sequence of any one of SEQ ID NOs: 4-20. In some embodiments, the present disclosure provides a method of treating or preventing a fibrotic disease or disorder in an elderly subject, wherein the method comprises administering to the elderly subject an effective amount of a modified Cav-1 peptide comprising an amino acid sequence of SEQ ID NO: 3. In some embodiments, the present disclosure provides a method of treating or preventing a fibrotic disease or disorder in an elderly subject, wherein the method comprises administering to the elderly subject an effective amount of a modified Cav-1 peptide comprising an amino acid sequence of SEQ ID NO: 8. In some embodiments, the present disclosure provides a method of treating or preventing a fibrotic disease or disorder in an elderly subject, wherein the method comprises administering to the elderly subject an effective amount of a modified Cav-1 peptide comprising at least one amino acid substitution, deletion of insertion relative to the amino acid sequence of FTTFTVT (SEQ ID NO: 3), wherein the modified Cav-1 peptide maintains the biological activity of Cav-1.

In some embodiments, the modified Cav-1 peptides or pharmaceutical compositions thereof are used in the treatment or prevention of a disease or disorder of the lungs in a subject. In some embodiments, the modified Cav-1 peptide is used to treat or prevent a lung disease or disorder such as, for example, acute lung injury (ALI), chronic lung injury, acute respiratory distress syndrome (ARDS), chronic obstructive pulmonary disease (COPD), asthma, interstitial lung disease, pulmonary fibrosis, pneumonia, hypersensitivity pneumonitis, bronchiolitis, sarcoidosis, scleroderma, or pulmonary infection. In some embodiments, the modified Cav-1 peptide is used to treat or prevent a lung disease or disorder in a subject that is elderly or of advanced age. In some embodiments, the elderly subject has interstitial lung disease, e.g., idiopathic pulmonary fibrosis.

In some embodiments, the modified Cav-1 peptides of the present disclosure are used to treat or prevent a pulmonary infection, e.g., a bacterial, viral, or fungal infection, in a subject. In some embodiments, the pulmonary infection causes one or more lung diseases or disorders in a subject, including but not limited to, ALI, ARDS, COPD, asthma, interstitial lung disease, lung fibrosis, pneumonia, hypersensitivity pneumonitis, bronchiolitis, sarcoidosis, and scleroderma.

In some embodiments, the modified Cav-1 peptides of the present disclosure are used to treat or prevent a bacterial infection in a subject. Examples of bacteria that cause pulmonary infections include, but are not limited to, Pseudomonas aeruginosa, Bacillus anthracis, Listeria monocytogenes, Staphylococcus aureus, Streptococcus pneumoniae, Haemophilus influenzae, Enterobacteriaceae, Nocardia, Actinomyces, Moraxella catarrhalis, Klebsiella pneumoniae, Chlamydia trachomatis, Chlamydophilia pneumoniae, Chlamydophilia psittaci, Coxiella burnetti, Salmenellosis, Yersina pestis, Mycobacterium leprae, Mycobacterium africanum, Mycobacterium asiaticum, Mycobacterium aviuin-intracellulaire, Mycobacterium chelonei, Mycobacterium abscessus, Mycobacterium fallax, Mycobacterium fortuitum, Mycobacterium kansasii, Mycobacterium leprae, Mycobacterium malmoense, Mycobacterium shimoidei, Mycobacterium simiae, Mycobacterium szulgai, Mycobacterium xenopi, Mycobacterium tuberculosis, Brucella melitensis, Brucella suis, Brucella abortus, Brucella canis, Legionella pneumonophilia, Francisella tularensis, Pneurnocystis carinii, Mycoplasma pneumoniae, or Burkholderia cepacia. In some embodiments, the bacterial infection causes pneumonia in the subject.

In some embodiments, the modified Cav-1 peptides of the present disclosure are used to treat or prevent a viral infection in a subject. In some embodiments, the modified Cav-1 peptide is used to treat or prevent an infection in a subject caused by a double-stranded DNA (dsDNA) virus, a single-stranded DNA (ssDNA) virus, a single-stranded RNA (ssRNA) virus, or a double-stranded RNA (dsRNA) virus. In some embodiments, the ssRNA virus is a positive-sense ssRNA virus (+ssRNA). In some embodiments, the ssRNA virus is a negative-sense ssRNA virus (−ssRNA). Examples of viruses that cause pulmonary infections include, but are not limited to, coronaviruses (e.g., SARS-CoV-1, SARS-CoV-2, or MERS-CoV), influenza, respiratory syncytial virus, metapneumovirus, bocavirus, parainfluenza, rhinovirus, enterovirus, norovirus, adenovirus, varicella-zoster virus, hantavirus, parechovirus, Epstein-Barr virus, herpes simplex virus, mimivirus, cytomegalovirus, torquetenovirus, and Middle East Respiratory Syndrome coronavirus. In some embodiments, the viral infection causes pneumonia in the subject. In some embodiments, the viral infection causes lung fibrosis in the subject. In some embodiments, the viral infection causes bronchiolitis in the subject. In some embodiments, the viral infection causes ALI or ARDS in the subject. In some embodiments, the viral infection causes interstitial lung disease in the subject. In some embodiments, the viral infection causes asthma in the subject. In some embodiments, the viral infection causes sarcoidosis in the subject. In some embodiments, the viral infection causes scleroderma in the subject.

In some embodiments, SARS-CoV-1 causes severe acute respiratory syndrome (SARS) in a subject. In some embodiments, the modified Cav-1 peptide is used to treat or prevent SARS in a subject. SARS is initially characterized by systemic symptoms of muscle pain, headache, and fever, followed in 2-14 days by the onset of respiratory symptoms, mainly cough, dyspnea, and pneumonia.

In some embodiments, MERS-CoV causes Middle East respiratory syndrome (MERS) in a subject. In some embodiments, the modified Cav-1 peptide is used to treat or prevent MERS in a subject. Clinical features of MERS range from asymptomatic or mild disease to acute respiratory distress syndrome and multiorgan failure resulting in death, especially in individuals with underlying comorbidities. No specific drug treatment exists for MERS and infection prevention and control measures are crucial to prevent spread in health-care facilities. See Zumla et al. Lancet 2015; 386(9997):995-1007.

In some embodiments, SARS-CoV-2 causes coronavirus disease 2019 (COVID-19) in a subject. In some embodiments, the modified Cav-1 peptide is used to treat or prevent an infection caused by SARS-CoV-2. In some embodiments, a variant of SARS-CoV-2 causes COVID-19 in a subject. In some embodiments, the modified Cav-1 peptide is used to treat or prevent an infection caused by a variant of SARS-CoV-2. In some embodiments, the modified Cav-1 peptide is used to treat or prevent an infection caused by a SARS-CoV-2 alpha variant. In some embodiments, the modified Cav-1 peptide is used to treat or prevent an infection caused by a SARS-CoV-2 beta variant. In some embodiments, the modified Cav-1 peptide is used to treat or prevent an infection caused by a SARS-CoV-2 gamma variant. In some embodiments, the modified Cav-1 peptide is used to treat or prevent an infection caused by a SARS-CoV-2 delta variant. In some embodiments, the modified Cav-1 peptide is used to treat or prevent an infection caused by a SARS-CoV-2 epsilon variant. In some embodiments, the modified Cav-1 peptide is used to treat or prevent an infection caused by a SARS-CoV-2 zeta variant. In some embodiments, the modified Cav-1 peptide is used to treat or prevent an infection caused by a SARS-CoV-2 eta variant. In some embodiments, the modified Cav-1 peptide is used to treat or prevent an infection caused by a SARS-CoV-2 theta variant. In some embodiments, the modified Cav-1 peptide is used to treat or prevent an infection caused by a SARS-CoV-2 iota variant. In some embodiments, the modified Cav-1 peptide is used to treat or prevent an infection caused by a SARS-CoV-2 kappa variant. In some embodiments, the modified Cav-1 peptide is used to treat or prevent an infection caused by a SARS-CoV-2 lambda variant. In some embodiments, the modified Cav-1 peptide is used to treat or prevent an infection caused by a SARS-CoV-2 mu variant. In some embodiments, the modified Cav-1 peptide is used to treat or prevent an infection caused by a SARS-CoV-2 omicron variant. In some embodiments, the SARS-CoV-2 variant is B.1.1.7 (also referred to as 501Y.V1 or VOC-202012/01), B.1.1.317, B.1.1.318, B.1.1.529, B.1.351 (also referred to as 501Y.V2), B.1.429, B1.427, B1.1.207, A.23.1, COH.20G/501Y, B.1.525, B.1.526, B.1.617, B.1.618, B. 1.621, C.37, P.1, P.2, or P.3, or a subvariant thereof, or a recombinant form thereof. See Konings et al., Variants of Interest and Concern naming scheme conducive for global discourse. Nature Microbiology (2021). In some embodiments, the subvariant of SARS-CoV-2 variant B.1.1.529 is BA.1 (B1.1.529.1), BA1.1 (B1.1.529.1.1), BA.2 (B1.1.529.2), BA.3 (B1.1.529.3), BA.4 (B1.1.529.4), or BA.5 (B1.1.529.5). In some embodiments, the subvariant of SARS-CoV-2 variant B.1.1.7 is Q.1, Q.2, Q.3, Q.4, Q.5, Q.6, Q.7, or Q.8. In some embodiments, the subvariant of SARS-CoV-2 variant B. 1.351 is B.1.351.1, B.1.351.2, B.1.351.3, B.1.351.4, or B.1.351.5. In some embodiments, the subvariant of SARS-CoV-2 variant P.1 is P.1.1, P.1.2, P.1.3, P.1.4, P.1.5, P.1.6, P.1.7, P.1.7.1, P.1.8, P.1.9, P.1.10, P.1.10.1, P.1.10.2, P.1.11, P.1.12, P.1.12.1, P.1.13, P.1.14, P.1.15, P. 1.16, P.1.17, or P.1.17.1. In some embodiments, the subvariant of SARS-CoV-2 variant B.1.617 is B.1.617.1, B.1.617.2, or B.1.617.3. In some embodiments, the subvariant of SARS-CoV-2 variant B.1.526 is B.1.526.1. In some embodiments, the subvariant of SARS-CoV-2 variant B. 1.621 is B.1.621.1, B.1.621.2, BB.1, or BB.2. In some embodiments, the subvariant of SARS-CoV-2 variant C.37 is C.37.1.

In some embodiments, the modified Cav-1 peptides of the present disclosure are used to treat or prevent a fungal infection in a subject. Examples of fungi that cause pulmonary infections include, but are not limited to, Candida (e.g., Candida albicans, Candida glabrata, Candida krusei), Aspergillus, Pneumocystis, Coccidioides (e.g., Coccidioides immitis, Coccidioides posadasii), Blastomyces (e.g., Blastomyces dermatitidis), Histoplasma (e.g., Histoplasma capsulatum), Cryptococcus (e.g., Cryptococcus neoformans, Cryptococcus gattii), Sporothrix (e.g., Sporothrix schenckii), Mucor, and Paracoccidioides. In some embodiments, the fungal infection causes pneumonia in the subject. In some embodiments, the fungal infection causes invasive pulmonary aspergillosis in the subject. In some embodiments, the fungal infection causes allergic asthma, allergic bronchopulmonary aspergillosis, or hypersensitivity pneumonitis in the subject. In some embodiments, the fungal infection causes ARDS. In some embodiments, the fungal infection causes pulmonary fibrosis in the subject. In some embodiments, the fungal infection causes pulmonary oedema in the subject.

In some embodiments, the modified Cav-1 peptides of the present disclosure are used to treat or prevent interstitial lung disease in a subject. Interstitial lung disease is a group of disorders that causes fibrosis and inflammation of the interstitium. In some embodiments, the interstitial lung disease is idiopathic pulmonary fibrosis, lymphangioleiomyomatosis, nonspecific interstitial pneumonia, idiopathic interstitial pneumonia, cryptogenic organizing pneumonia, acute interstitial pneumonia, respiratory bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced interstitial lung disease, or occupational interstitial lung disease. In some embodiments, the interstitial lung disease is idiopathic pulmonary fibrosis.

In some embodiments, the modified Cav-1 peptides of the present disclosure are used to treat or prevent acute lung injury (ALI) in a subject. ALI is a disorder of acute inflammation that causes disruption of the lung endothelial and epithelial barriers. ALI may be the result of inhalation injury or a consequence of systemic disease, such as sepsis or severe hypovolemic shock. In some embodiments, the ALI is chemical-induced ALI. In some embodiments, the ALI is inhalational smoke induced acute lung injury (ISALI). In some embodiments, the ALI is ARDS.

In some embodiments, the modified Cav-1 peptides of the present disclosure are used to treat or prevent ARDS in a subject. ARDS is the most severe form of ALI and is distinguished by the severity of the oxygenation deficit. ARDS is a life-threatening type of lung injury that occurs when fluid builds up in the tiny, elastic air sacs (alveoli) in the lungs. The fluid in the alveoli prevents the lungs from filling up with oxygen resulting in less oxygen reaching the bloodstream and a difficulty in breathing.

In some embodiments, the modified Cav-1 peptide is used to treat or prevent cystic fibrosis (CF) in a subject. CF is an inherited disease of the exocrine glands and exocrine sweat glands which primarily affects the digestive and respiratory systems. This disease is usually characterized by chronic respiratory infections, pancreatic insufficiency, abnormally viscid mucus secretions and premature death. CF is characterized by progressive airflow obstruction. Subsets of individuals with CF also develop airway hyper-responsiveness to inhaled cholinergic agonists (Weinberger, 2002 and Mitchell et al., 1978) and reversibility of airflow limitation in response to bronchodilators (van Haren et al., 1991 and van Haren et al., 1992). The presence of bronchial hyper-responsiveness and airway obstruction suggest a possible shared etiology of disease between CF and other diseases of airway narrowing such as asthma or COPD where airway smooth muscle dysfunction is thought to contribute to the disease processes.

In some embodiments, the modified Cav-1 peptide is used to treat or prevent COPD in a subject. COPD is a term used to classify two major airflow obstruction disorders: chronic bronchitis and emphysema. Chronic bronchitis is inflammation of the bronchial airways. The bronchial airways connect the trachea with the lungs. When inflamed, the bronchial tubes secrete mucus, causing a chronic cough. In emphysema, the alveolar sacs are overinflated as a result of damage to the elastin skeleton of the lung. Inflammatory cells in emphysematous lung release elastase enzymes, which degrade or damage elastin fibers within the lung matrix. Emphysema has a number of causes, including smoking, exposure to environmental pollutants, alpha-one antitrypsin deficiency, and aging.

In some embodiments, the modified Cav-1 peptides disclosed herein are used to treat or prevent bronchiolitis in a subject. Bronchiolitis is most commonly caused by viral lower respiratory tract infections, and primarily characterized by acute inflammation, edema, necrosis of epithelial cells lining small airways, and increased mucus production (Ralston et al., 2014). Signs and symptoms typically begin with rhinitis and cough, which may progress to tachypnea, wheezing, rales, use of accessory muscles, and/or nasal flaring.

In some embodiments, the modified Cav-1 peptides disclosed herein are used to treat or prevent bronchiolitis obliterans in a subject. Bronchiolitis obliterans is a progressive airflow reduction as a result of abnormal remodeling of the small airways in the lungs (Meyer et al., 2014). Bronchiolitis obliterans is a major complication of lung transplantations, and is often used to describe a delayed allograft dysfunction that results in persistent decline in forced expiratory volume and force that is not caused by other known causes (Meyer et al., 2014).

In some embodiments, the modified Cav-1 peptides disclosed herein are used to treat or prevent asthma in a subject. The term “asthma” may refer to acute asthma, chronic asthma, intermittent asthma, mild persistent asthma, moderate persistent asthma, severe persistent asthma, chronic persistent asthma, mild to moderate asthma, mild to moderate persistent asthma, mild to moderate chronic persistent asthma, allergic (extrinsic) asthma, non-allergic (intrinsic) asthma, nocturnal asthma, bronchial asthma, exercise induced asthma, occupational asthma, seasonal asthma, silent asthma, gastroesophageal asthma, idiopathic asthma and cough variant asthma. During asthma, the airways are persistently inflamed and may occasionally spasm.

In some embodiments, the modified Cav-1 peptides disclosed herein are used to treat or prevent hypersensitivity pneumonitis in a subject. Hypersensitivity pneumonitis is a complex syndrome caused by the inhalation of a variety of antigens in susceptible and sensitized individuals. These antigens are found in the environment, mostly derived from bird proteins and fungi. Hypersensitivity pneumonitis is characterized by an exaggerated humoral and cellular immune response affecting the small airways and lung parenchyma. Hypersensitivity pneumonitis can be classified into acute, chronic non-fibrotic and chronic fibrotic forms. Acute hypersensitivity pneumonitis results from intermittent, high-level exposure to the inducing antigen, usually within a few hours of exposure, whereas chronic hypersensitivity pneumonitis mostly originates from long-term, low-level exposure (usually to birds or molds in the home), is not easy to define in terms of time, and may occur within weeks, months or even years of exposure. Some patients with fibrotic hypersensitivity pneumonitis may evolve to a progressive phenotype, even with complete exposure avoidance. See Costabel et al., Nature Reviews Disease Primers 2020; 6(65).

In some embodiments, the modified Cav-1 peptide is used to treat or prevent systemic sclerosis or scleroderma in a subject. Systemic sclerosis is a systemic autoimmune disease that is characterized by endothelial dysfunction resulting in a small-vessel vasculopathy, fibroblast dysfunction with resultant excessive collagen production and fibrosis, and immunological abnormalities. The classification of systemic sclerosis is subdivided based on the extent of skin involvement into diffuse cutaneous sclerosis, limited cutaneous sclerosis or systemic sclerosis sine scleroderma. While virtually any organ system may be involved in the disease process, fibrotic and vascular pulmonary manifestations of systemic sclerosis, including interstitial lung disease and pulmonary hypertension, are the leading cause of death. While certain pulmonary manifestations may occur more commonly in a subset of systemic sclerosis (i.e. ILD is more common in diffuse cutaneous sclerosis, while pulmonary hypertension is more common in limited cutaneous sclerosis), all of the known pulmonary manifestations reported have been described in each of the subsets of disease. Pulmonary disease can even occur in systemic sclerosis with no skin involvement (an entity known as scleroderma sine scleroderma). See Solomon et al., Eur Respir Rev 2013; 22(127):6-19.

In some embodiments, the modified Cav-1 peptide is used to treat or prevent sarcoidosis in a subject. Sarcoidosis is a multisystem disorder that is characterized by noncaseous epithelioid cell granulomas, which may affect almost any organ. Thoracic involvement is common and accounts for most of the morbidity and mortality associated with the disease. Thoracic abnormalities are observed in approximately 90% of patients with sarcoidosis, and an estimated 20% develop chronic lung disease leading to pulmonary fibrosis. Pulmonary sarcoidosis may manifest with various patterns: Bilateral hilar lymph node enlargement is the most common finding, followed by interstitial lung disease. The most typical findings of pulmonary involvement are micronodules with a perilymphatic distribution, fibrotic changes, and bilateral perihilar opacities. Atypical manifestations, such as mass-like or alveolar opacities, honeycomb-like cysts, miliary opacities, mosaic attenuation, tracheobronchial involvement, and pleural disease, and complications such as aspergillomas, also may be seen. See Criado et al., Chest Imaging 2010; 30(6).

In some embodiments, the present disclosure provides a method of treating or preventing a lung disease or disorder in an elderly subject, wherein the method comprises administering to the elderly subject an effective amount of a modified Cav-1 peptide or pharmaceutical composition thereof. In some embodiments, the present disclosure provides a method of treating or preventing a lung disease or disorder in an elderly subject, wherein the method comprises administering to the elderly subject an effective amount of a modified Cav-1 peptide comprising an amino acid sequence of any one of SEQ ID NOs: 2-111. In some embodiments, the present disclosure provides a method of treating or preventing a lung disease or disorder in an elderly subject, wherein the method comprises administering to the elderly subject an effective amount of a modified Cav-1 peptide comprising an amino acid sequence of any one of SEQ ID NOs: 4-20. In some embodiments, the present disclosure provides a method of treating or preventing a lung disease or disorder in an elderly subject, wherein the method comprises administering to the elderly subject an effective amount of a modified Cav-1 peptide comprising an amino acid sequence of SEQ ID NO: 3. In some embodiments, the present disclosure provides a method of treating or preventing a lung disease or disorder in an elderly subject, wherein the method comprises administering to the elderly subject an effective amount of a modified Cav-1 peptide comprising an amino acid sequence of SEQ ID NO: 8. In some embodiments, the present disclosure provides a method of treating or preventing a lung disease or disorder in an elderly subject, wherein the method comprises administering to the elderly subject an effective amount of a modified Cav-1 peptide comprising at least one amino acid substitution, deletion of insertion relative to the amino acid sequence of FTTFTVT (SEQ ID NO: 3), wherein the modified Cav-1 peptide maintains the biological activity of Cav-1. In some embodiments, the method of administering the modified Cav-1 peptide further comprises nebulizing a solution comprising the modified Cav-1 peptide.

The present invention contemplates all modes of administration, dosing, or frequency of dosing adequate to treat or prevent the disease or disorder in a subject. Effective doses may also be extrapolated from dose-response curves derived from in vitro or animal model test bioassays or systems.

In some embodiments, the modified Cav-1 peptide or pharmaceutical composition thereof is administered intravenously, intrathecally, intradermally, transdermally, intrathecally, intraarterially, intraperitoneally, intranasally, intravaginally, intravesicular, intraarticular, intralesional, intrarectally, intramuscularly, subcutaneously, mucosally, orally, topically, locally, by inhalation (e.g., inhalation of a nebulized or dry powder formulation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference). The choice of injection volume and needle size may be chosen by the person of ordinary skill in the art based on site of injection, syringeability and injectability, which includes considering the viscosity of the solution or suspension to be injected and drug concentration, pH, and osmolality. In some instances, the particle size of the active agent can be chosen in order to provide a desired rate of dissolution upon administration (e.g., by subcutaneous injection).

In some embodiments, the modified Cav-1 peptides or pharmaceutical compositions thereof is administered systemically. In some embodiments, the modified Cav-1 peptide or pharmaceutical composition thereof is administered intravenously, intrathecally, subcutaneously, and/or intraperitoneally.

In some embodiments, the modified Cav-1 peptide or pharmaceutical composition thereof is delivered locally to the airway of a subject, such as administration of a nebulized formulation using a nebulizer or a dry powder formulation using a dry powder inhaler. In some embodiments, the modified Cav-1 peptide or pharmaceutical composition thereof is administered to the lungs of an elderly subject using a nebulizer. In some embodiments, the modified Cav-1 peptide or pharmaceutical composition thereof is administered to the lungs of an elderly subject using a dry powder inhaler. In some embodiments, the modified Cav-1 peptide or pharmaceutical composition thereof is administered intranasally, intrabronchially, intrapleurally, intratracheally, or via inhalation to an elderly subject.

In some embodiments, the modified Cav-1 peptide or pharmaceutical composition thereof is administered to a subject in a single dose or in multiple doses. When multiple doses are administered, the doses may be separated from one another by, for example, one hour, three hours, six hours, eight hours, twelve hours, one day, two days, three days, four days, five days, six days, one week, two weeks, three weeks, one month, two months, three months, four months, five months, six months, one year, or any value or range therebetween. In some embodiments, the modified Cav-1 peptide or pharmaceutical composition thereof is administered, for example, once every 2 weeks, once every 3 weeks, once every 4 weeks, once every 5 weeks, once every 6 weeks, once every 7 weeks, once every 8 weeks, once every 10 weeks, once every 15 weeks, once every 20 weeks, or more. It is to be understood that, for any particular subject, specific dosage regimens should be adjusted over time according to the individual need and the professional judgment of the person administering or supervising the administration of the compositions. For example, the dosage of the modified Cav-1 peptide or pharmaceutical composition thereof can be increased if the lower dose does not provide sufficient therapeutic activity.

In some embodiments, the modified Cav-1 peptide or pharmaceutical composition thereof is administered to a subject at a dose of about 0.0001 mg/kg to about 1,000 mg/kg. In some embodiments, the modified Cav-1 peptide or pharmaceutical composition thereof is administered to a subject at a dose of about 0.0001 mg/kg to about 0.01 mg/kg. In some embodiments, the modified Cav-1 peptide or pharmaceutical composition thereof is administered to a subject at a dose of about 0.01 mg/kg to about 1 mg/kg. In some embodiments, the modified Cav-1 peptide or pharmaceutical composition thereof is administered to a subject at a dose of about 1 mg/kg to about 100 mg/kg. In some embodiments, the modified Cav-1 peptide or pharmaceutical composition thereof is administered to a subject at a dose of about 1 mg/kg to about 50 mg/kg. In some embodiments, the modified Cav-1 peptide or pharmaceutical composition thereof is administered to a subject at a dose of about 1 mg/kg to about 25 mg/kg. In some embodiments, the modified Cav-1 peptide or pharmaceutical composition thereof is administered to a subject at a dose of about 1 mg/kg to about 10 mg/kg. In some embodiments, the modified Cav-1 peptide or pharmaceutical composition thereof is administered to a subject at a dose of about 10 mg/kg to about 25 mg/kg. In some embodiments, the modified Cav-1 peptide or pharmaceutical composition thereof is administered to a subject at a dose of about 25 mg/kg to about 50 mg/kg. In some embodiments, the modified Cav-1 peptide or pharmaceutical composition thereof is administered to a subject at a dose of about 50 mg/kg to about 75 mg/kg. In some embodiments, the modified Cav-1 peptide or pharmaceutical composition thereof is administered to a subject at a dose of about 75 mg/kg to about 100 mg/kg. In some embodiments, the modified Cav-1 peptide or pharmaceutical composition thereof is administered to a subject at a dose of about 0.0001 mg/kg, about 0.01 mg/kg, about 0.01 mg/kg, about 0.1 mg/kg, about 1 mg/kg, about 5 mg/kg, about 10 mg/kg, about 25 mg/kg, about 50 mg/kg, about 100 mg/kg, about 500 mg/kg, or about 1,000 mg/kg.

In some embodiments, the modified Cav-1 peptide or pharmaceutical composition thereof is administered to a subject at a dose of about 0.0001 g/kg to about 1,000 g/kg. In some embodiments, the modified Cav-1 peptide or pharmaceutical composition thereof is administered to a subject at a dose of about 0.0001 g/kg to about 0.01 g/kg. In some embodiments, the modified Cav-1 peptide or pharmaceutical composition thereof is administered to a subject at a dose of about 0.01 g/kg to about 1 g/kg. In some embodiments, the modified Cav-1 peptide or pharmaceutical composition thereof is administered to a subject at a dose of about 1 g/kg to about 100 g/kg. In some embodiments, the modified Cav-1 peptide or pharmaceutical composition thereof is administered to a subject at a dose of about 1 g/kg to about 50 g/kg. In some embodiments, the modified Cav-1 peptide or pharmaceutical composition thereof is administered to a subject at a dose of about 1 g/kg to about 25 g/kg. In some embodiments, the modified Cav-1 peptide or pharmaceutical composition thereof is administered to a subject at a dose of about 1 g/kg to about 10 g/kg. In some embodiments, the modified Cav-1 peptide or pharmaceutical composition thereof is administered to a subject at a dose of about 10 mg/kg to about 25 mg/kg. In some embodiments, the modified Cav-1 peptide or pharmaceutical composition thereof is administered to a subject at a dose of about 25 g/kg to about 50 g/kg. In some embodiments, the modified Cav-1 peptide or pharmaceutical composition thereof is administered to a subject at a dose of about 50 g/kg to about 75 g/kg. In some embodiments, the modified Cav-1 peptide or pharmaceutical composition thereof is administered to a subject at a dose of about 75 g/kg to about 100 g/kg. In some embodiments, the modified Cav-1 peptide or pharmaceutical composition thereof is provided at a dose of about 0.0001 g/kg, about 0.01 g/kg, about 0.01 g/kg, about 0.1 g/kg, about 1 g/kg, about 5 g/kg, about 10 g/kg, about 25 g/kg, about 50 g/kg, about 100 g/kg, about 500 g/kg, or about 1,000 g/kg.

In some embodiments, the total or complete dose of a modified Cav-1 peptide or pharmaceutical composition thereof administered to a subject is between about 1 mg to about 100 mg, such as between about 20 mg to about 100 mg, between about 50 mg to about 100 mg, between about 10 mg to about 20 mg, between about 20 mg to about 40 mg, between about 50 mg to about 70 mg, or between about 80 mg to about 90 mg.

In some embodiments, dosages of the modified Cav-1 peptide or pharmaceutical composition thereof for a particular subject are determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to a subject is sufficient to provide a beneficial therapeutic response in the subject over time, or, e.g., to reduce symptoms, or other appropriate activity, depending on the application. The dose is determined by the efficacy of the particular formulation, and the activity, stability and/or serum half-life of the modified Cav-1 peptides disclosed herein and the condition of the subject, as well as the body weight or surface area of the subject to be treated.

In some embodiments, a subject is administered a dose of the modified Cav-1 peptide or pharmaceutical composition thereof once per day for the treatment or prevention of any one of the diseases or conditions described herein. In some embodiments, the subject is elderly or of advanced age. In some embodiments, the single dose is between about 0.2 mg/kg and about 250 mg/kg, such as between about 1 mg/kg to about 10 mg/kg, between about 10 mg/kg and about 25 mg/kg, between about 25 mg/kg to about 50 mg/kg, between about 50 mg/kg to about 75 mg/kg, between about 75 mg/kg to about 100 mg/kg, for example, via lung instillation (e.g., inhalation). Such a dose can be administered daily for anywhere from about 3 days to one or more weeks or at any frequency as disclosed herein. Chronic administration of the modified Cav-1 peptide or pharmaceutical composition thereof is also possible, although the dose may need to be adjusted downward as is well-understood in the art. The foregoing ranges are, however, suggestive, as the number of variables in an individual treatment regime is large, and considerable excursions from these preferred values are expected.

In some embodiments, the modified Cav-1 peptide or pharmaceutical composition thereof is administered continuously to a subject. For continuous administration, e.g., by a pump system such as an osmotic pump, a total dosage for a time course of about 1-2 weeks is preferably in the range of 1 mg/kg to 1 g/kg, preferably 20-300 mg/kg, more preferably 50-200 mg/kg. After such a continuous dosing regimen, the total concentration of the active compound is preferably in the range of about 0.5 μM to about 50 μM, preferably about 1 μM to about 10 μM.

In some embodiments, the modified Cav-1 peptide or pharmaceutical composition thereof is administered on a routine schedule. As used herein, a routine schedule refers to a predetermined designated period of time. The routine schedule may encompass periods of time which are identical or which differ in length, as long as the schedule is predetermined. For instance, the routine schedule may involve administration once per day, twice per day, once every two days, once every three days, once every four days, once every five days, once every six days, once per week, once every two weeks, once every three weeks, once per month, once every two months, once every three months, once every six months, or any set number of days, weeks, or months there-between. In some embodiments, the modified Cav-1 peptide or pharmaceutical composition thereof is administered on a twice daily basis for the first week, followed by a daily basis for several months. In some embodiments, the modified Cav-1 peptide or pharmaceutical composition thereof is administered once per day. In some embodiments, the modified Cav-1 peptide or pharmaceutical composition thereof is administered less than once per day, such as every other day, every third day, or once per week.

In some embodiments, the modified Cav-1 peptide is administered to a subject with a kidney disease or disorder for at least about 2 weeks, at least about 3 weeks, at least about 4 weeks, at least about 5 weeks, at least about 6 weeks, at least about 2 months, at least about 3 months, at least about 4 months, at least about 5 months, at least about 6 months, at least about 1 year, or any set number of weeks or months there-between. In some embodiments, the modified Cav-1 peptide is administered to a subject with a kidney disease or disorder for at least about 2 weeks. In some embodiments, the modified Cav-1 peptide is administered to a subject with a kidney disease or disorder for at least about 4 weeks.

In some embodiments, the modified Cav-1 peptide is administered to a subject with a kidney disease or disorder once per day for at least about 2 weeks, at least about 3 weeks, at least about 4 weeks, at least about 5 weeks, at least about 6 weeks, at least about 2 months, at least about 3 months, at least about 4 months, at least about 5 months, at least about 6 months, at least about 1 year, or any set number of weeks or months there-between. In some embodiments, the modified Cav-1 peptide is administered to a subject with a kidney disease or disorder once per day for at least about 2 weeks. In some embodiments, the modified Cav-1 peptide is administered to a subject with a kidney disease or disorder once per day for at least about 4 weeks.

In some embodiments, the modified Cav-1 peptide or pharmaceutical composition thereof is provided in a unit dosage form (e.g., pre-divided dose), such as in a capsule, blister or a cartridge. In some embodiments, the unit dose comprises at least 1 mg of the modified Cav-1 peptide, such as at least about 5 mg, at least about 10 mg, at least about 15 mg, or at least about 20 mg of the modified Cav-1 peptide per dose. In some embodiments, the unit dose is about 1 mg to about 10 mg (e.g., about 5 mg) of the modified Cav-1 peptide. In some embodiments, the unit dosage form does not comprise the administration or addition of any excipient and is merely used to hold the powder for inhalation (i.e., the capsule, blister, or cartridge is not administered). In some embodiments, more than one of the unit dose forms is administered to a subject. For example, in the case of a dry powder inhaler, the modified Cav-1 peptide is provided in unit dose capsules and more than one unit dose capsules (e.g., 3-4) can be administered to a subject by inhalation. In some embodiments, the modified Cav-1 peptide is administered in a high emitted dose, such as at least about 10 mg, preferably at least about 15 mg, even more preferably at least about 20 mg. In some embodiments, administration of milled modified Cav-1 peptide results in a high fine particle dose into the deep lung such as greater than about 5 mg. Preferably, the fine particle dose into the deep lung is at least about 10 mg, even more preferably at least about 15 mg. In some embodiments, the particle dose is produced from 1, 2, 3, 4 or 5 or more capsules comprising doses of a peptide of the embodiments. In some embodiments, the fine particle dose is at least about 50%, at least about 60%, at least about 65%, at least about 70%, at least about 75%, or at least about 80% of the emitted dose.

In some embodiments, changes in inhalation pressure result in a change in emitted dose. In some embodiments, changes in inhalation pressure of about 3 kPa, such as from about 4 kPa to about 1 kPa, result in a reduction of emitted dose of less than about 25%, such as about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5% or less. In some embodiments, changes in inhalation pressure result in a change in fine particle dose. In some embodiments, changes in inhalation pressure of about 3 kPa, such as from about 4 kPa to about 1 kPa result in a reduction of fine particle dose of less than about 15%, such as about 14%, about 13%, about 12%, about 11%, about 10%, about 9%, about 8%, about 7%, about 6%, about 5% or less.

In some embodiments, the modified Cav-1 peptide or pharmaceutical composition thereof is administered in combination, simultaneously or sequentially with at least one additional therapeutic agent for the treatment or prevention of a disease or disorder of the kidneys in a subject. In some embodiments, the disease is chronic kidney disease. The additional therapeutic agents include, but are not limited to, angiotensin-converting enzyme (ACE) inhibitors, such as, Capoten® (captopril), Vasotec® (enalapril), Monopril® (fosinopril), Prinivil® or Zestril®(lisinopril), or Altace® (ramipril); angiotensin II receptor (ARB) inhibitors, such as Edarbi® (azilsartan), Teveten® (eprosartan), Avapro® (irbesartan), Cozaar® (losartan), Benicar® (olmesartan), or Diovan® (valsartan); Farxiga® (dapagliflozin); Aranesp® (darbepoetin alpha); and/or Procrit® or Epogen® (erythropoietin).

In some embodiments, the modified Cav-1 peptide or pharmaceutical composition thereof is administered in combination with dialysis for the treatment of a disease or disorder of the kidneys in a subject. In some embodiments, the modified Cav-1 peptide or pharmaceutical composition thereof is administered in combination with dialysis for the treatment of chronic kidney disease. In some embodiments, the dialysis is hemodialysis. In some embodiments, the dialysis is peritoneal dialysis.

In some embodiments, the modified Cav-1 peptide or pharmaceutical composition thereof is administered alone or in combination with at least one additional therapeutic agent in a subject. In some embodiments, the modified Cav-1 peptide or pharmaceutical composition thereof is administered in combination, simultaneously or sequentially with at least one additional therapeutic agent for the treatment or prevention of a kidney disease or disorder in a subject. In some embodiments, the modified Cav-1 peptide or pharmaceutical composition thereof is administered in combination, simultaneously or sequentially with at least one additional therapeutic agent for the treatment or prevention of a fibrotic disease or disorder in an elderly subject. The additional therapeutic agents include, but are not limited to, an non-steroidal anti-inflammatory drug (NSAID), steroid, disease-modifying antirheumatic drug (DMARD), immunosuppressive, biologic response modulators, bronchodilator or antifibrotic agent such as pirfenedone, an agent whose antifibrotic mechanism of action is not fully understood but may involve blockade of TGF-beta, nintedanib, a broad tyrosine kinase blocker or any other antifibrotic agent. Suitable NSAIDS are selected from the non-selective cyclooxygenase (COX)-inhibitors acetylsalicyclic acid, mesalazin, ibuprofen, naproxen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, indomethacin, sulindac, tolmetin, zomepirac, nabumetone, diclofenac, fenclofenac, alclofenac, bromfenac, ibufenac, aceclofenac, acemetacin, fentiazac, clidanac, etodolac, oxpinac, mefenamic acid, meclofenamic acid, flufenamic acid, nifluminic acid, tolfenamic acid, diflunisal, flufenisal, piroxicam, tenoxicam, lornoxicam and nimesulide and the pharmaceutically acceptable salts thereof, the selective COX 2-inhibitors meloxicam, celecoxib and rofecoxib and the pharmaceutically acceptable salts thereof. Suitable steroids are prednisone, prednisolone, methylprednisolone, dexamethasone, budenoside, fluocortolone and triamcinolone. Suitable DMARDs are sulfasalazine, olsalazine, chloroquin, gold derivatives (auranofin), D-penicillamine and cytostatics such as methotrexate and cyclophosphamide. Suitable immunosuppressives are cyclosporine A and derivatives thereof, mycophenolatemofetil, FK 506 (also known as tacrolimus and fugimycin), muromonab-CD3 (Orthoclone OKT-3®), anti-thymocyte globulin (ATG), 15-desoxyspergualin, mizoribine, misoprostol, rapamycin, reflunomide and azathioprine. Suitable biologic response modifiers are interferon β, anti-TNF-α antibody (etanercept), IL-10, anti-CD3 antibody or anti-CD25 antibody. Suitable bronchodilators are ipratropiumbromide, oxytropiumbromide, tiotropiumbromide, epinephrinehydrochloride, salbutamole, terbutalinsulfate, fenoterolhydrobromide, salmeterole and formoterole. In such combinations each active ingredient can be administered either in accordance with its usual dosage range or a dose below its usual dosage range. The dosage for the combined NSAIDs, steroids, DMARDs, immunosuppressives and biologic response modifiers is appropriately 1/50 of the lowest dose normally recommended up to 1/1 of the normally recommended dosage, preferably 1/20 to ½ and more preferably 1/10 to ⅕. The normally recommended dose for the combined drug should be understood to be the dose disclosed, for example, in Rote Liste® 2002, Edition Cantor Verlag Aulendorf, Germany, or in Physician's Desk Reference.

In some embodiments, the modified Cav-1 peptide or pharmaceutical composition thereof is administered in combination, simultaneously or sequentially with at least one additional therapeutic agent for the treatment or prevention of a kidney disease or disorder in a subject. In some embodiments, the modified Cav-1 peptide or pharmaceutical composition thereof is administered in combination, simultaneously or sequentially with at least one additional therapeutic agent to treat a pathogen or pathogen-induced lung injury in an elderly subject. Additional therapeutic agents include, but are not limited to, chloroquine, hydroxychloroquine, type I interferon, anti-virals, antibiotics, remdesivir, favipiravir, lopinavir, and ritonavir.

Hydroxychloroquine is a chemical derivative of chloroquine which features a hydroxyethyl group instead of an ethyl group. Hydroxychloroquine has been classified as an effective anti-malarial medication and has shown efficacy in treating systemic lupus erythematosus as well as rheumatoid arthritis and Sjögren's Syndrome. While hydroxychloroquine has been known for some time to increase lysosomal pH in antigen presenting cells, its mechanism of action in inflammatory conditions has been only recently elucidated and involves blocking the activation of toll-like receptors to on plasmacytoid dendritic cells. Hydroxychloroquine has shown efficacy in treating RNA viruses, including hepatitis C. Hydroxychloroquine may be administered at a dose of 600 mg per day.

Human type I interferons (IFNs) are a large subgroup of interferon proteins that help regulate the activity of the immune system. The mammalian types are designated IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin). Type I interferons have shown efficacy against the replication of various viruses, included Zika virus, chikungunya virus, flaviviruses, and hepatitis C virus. “Interferon compounds” include interferon-alpha, interferon-alpha analogues, interferon-alpha derivatives, interferon-alpha conjugates, interferon beta, interferon-beta analogues, interferon-beta derivatives, interferon-beta conjugates and mixtures thereof. The whole protein or its fragments can be fused with other peptides and proteins such as immunoglobulins and other cytokines. Interferon-alpha and interferon-beta conjugates may represent, for example, a composition comprising interferon-beta coupled to a non-naturally occurring polymer comprising a polyalkylene glycol moiety. Preferred interferon compounds include Roferon® (interferon alpha-2a), Intron® (interferon alpha-2b), Alferon® (interferon alpha-n3), Infergen® (interferon alfacon-1), Omniferon® (interferon alpha), interferon alfacon-1, interferon-alpha, interferon-alpha analogues, pegylated interferon-alpha, polymerized interferon-alpha, dimerized interferon-alpha, interferon-alpha conjugated to carriers, interferon-alpha as oral inhalant, interferon-alpha as injectable compositions, interferon-alpha as a topical composition, Roferon® (interferon alpha-2a) analogues, Intron® (interferon alpha-2b) analogues, Alferon® (interferon alpha-n3) analogues, and Infergen® (interferon alfacon-1) analogues, Omniferon® (interferon alpha) analogues, interferon alfacon-1 analogues, interferon beta, Avonex™ (interferon beta-1a), Betaseron™ (interferon beta-1b), Betaferon™ (interferon beta-1b), Rebif™ (interferon beta-1a), interferon-beta analogues, pegylated interferon-beta, polymerized interferon-beta, dimerized interferon-beta, interferon-beta conjugated to carriers, interferon-beta as oral inhalant, interferon-beta as an injectable composition, interferon-beta as a topical composition, Avonex™ (interferon beta-1a) analogues, Betaseron™ (interferon beta-1b) analogues, Betaferon™ (interferon beta-1b) analogues, and Rebif™ (interferon beta-1a) analogues. Alternatively, agents that induce interferon-alpha or interferon-beta production or mimic the action of interferon-alpha or interferon-beta may also be employed. Interferon inducers include tilorone, poly (I)-poly (C), imiquimod, cridanimod, bropirimine.

### EXAMPLES

### Example 1: Efficacy of Modified Cav-1 Peptides in Chronic Kidney Disease

The objective of this study was to evaluate the efficacy of the modified Cav-1 peptide APi2355 in ameliorating kidney fibrosis and disease using the Col4a3−/− mouse model of Alport syndrome. Alport syndrome mice (knockouts) have reduced Cav-1 expression in glomerulae tubules and develop glomerular fibrosis.

Study 1: Two Weeks of Treatment with Saline or APi2355 in Col4a3−/− Mouse Model of Alport Syndrome

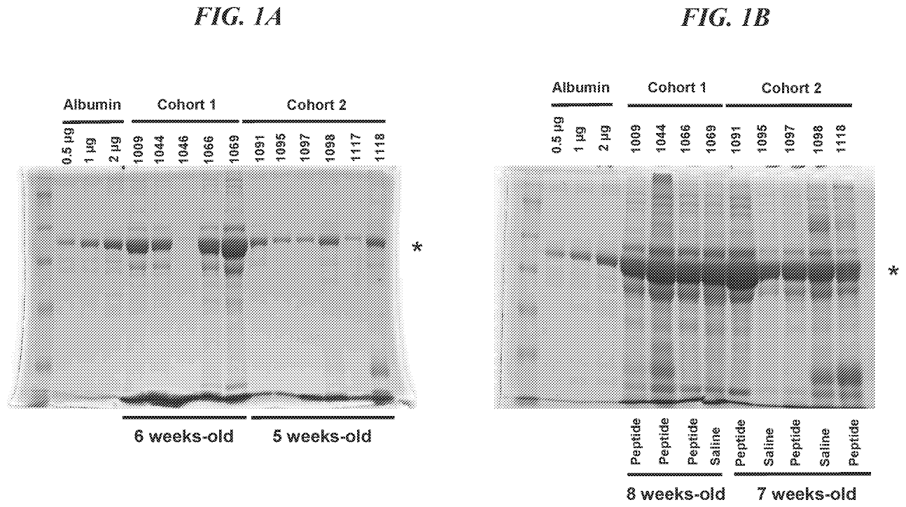

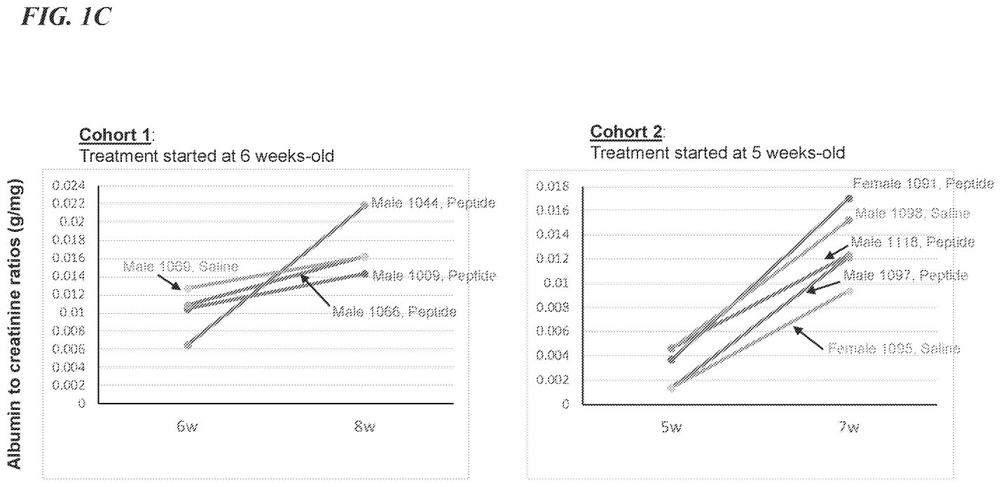

Col4a3−/− mice on the 129S1/SvlmJ strain background exhibit rapid progression of end stage renal disease by 11-13 weeks of age and were used in this study to assess efficacy of APi2355 in chronic kidney disease. Col4a3−/− on the 129S1/SvlmJ develop end-stage renal disease at a faster rate compared to Col4a3−/− mice on the C57BL/6 background. Five week-old (Cohort 2) and 6 week-old (Cohort 1) Col4a3−/− mice on a 129S1/SvlmJ background were intraperitoneally injected daily with APi2355 (SEQ ID NO: 8, 0.0022 mg/g body weight) or with normal saline for 2 weeks. Urine was collected prior to treatment and on the day of sacrifice to monitor for glomerular permselectivity by measuring albumin to creatinine (A/C) ratios. Blood was collected on the day of sacrifice to monitor for glomerular filtration rate by blood urea nitrogen (BUN) assay and kidney tissue was collected following sacrifice to assess for renal injury and fibrosis. Mouse identification numbers were used to differentiate treatment groups. (e.g., mice identification nos. 1009, 1044, 1066, and 1069 for Cohort 1 as shown in FIG. 1A).

FIG. 1A shows a urinary protein gel from Cohort 1 and Cohort 2 Col4a3−/− mice prior to treatment with saline or APi2355 peptide and FIG. 1B shows a urinary protein gel from Cohort 1 and Cohort 2 Col4a3−/− mice after 2 weeks of daily intraperitoneal injections with APi2355 peptide or saline. FIG. 1C shows albumin to creatinine ratios (g/mg) in the urine of Cohort 1 and Cohort 2 Col4a3−/− mice before and after 2 weeks of treatment with saline or APi2355 peptide.

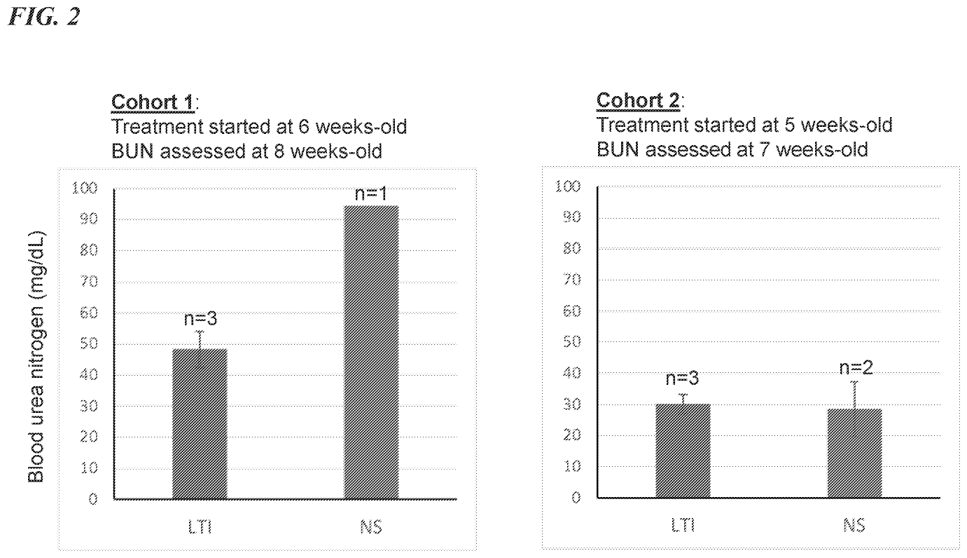

FIG. 2 shows urea nitrogen levels in the blood of Cohort 1 and Cohort 2 Col4a3−/− mice following treatment with saline or APi2355 peptide.

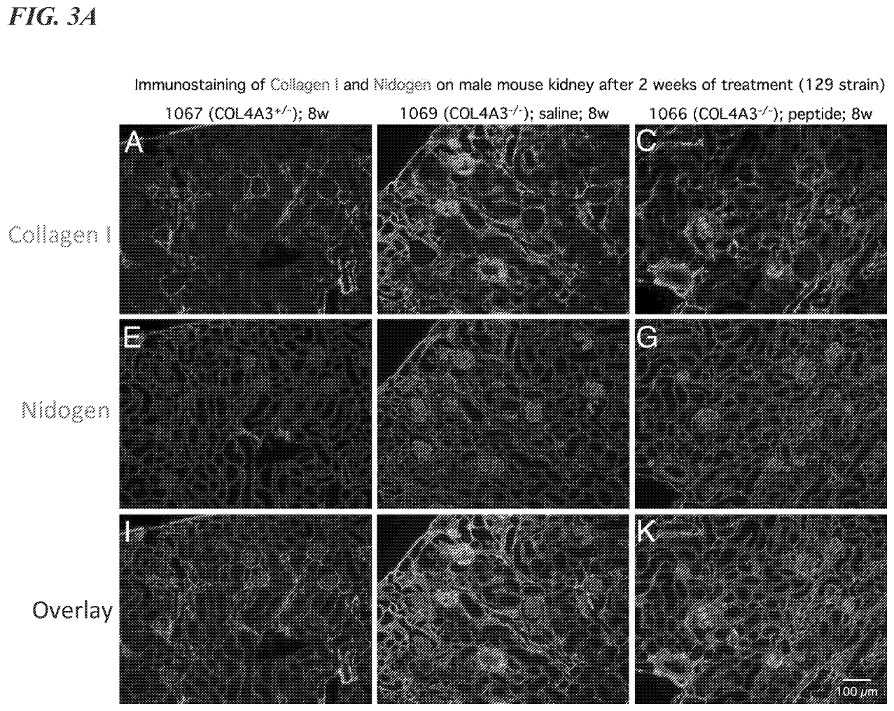

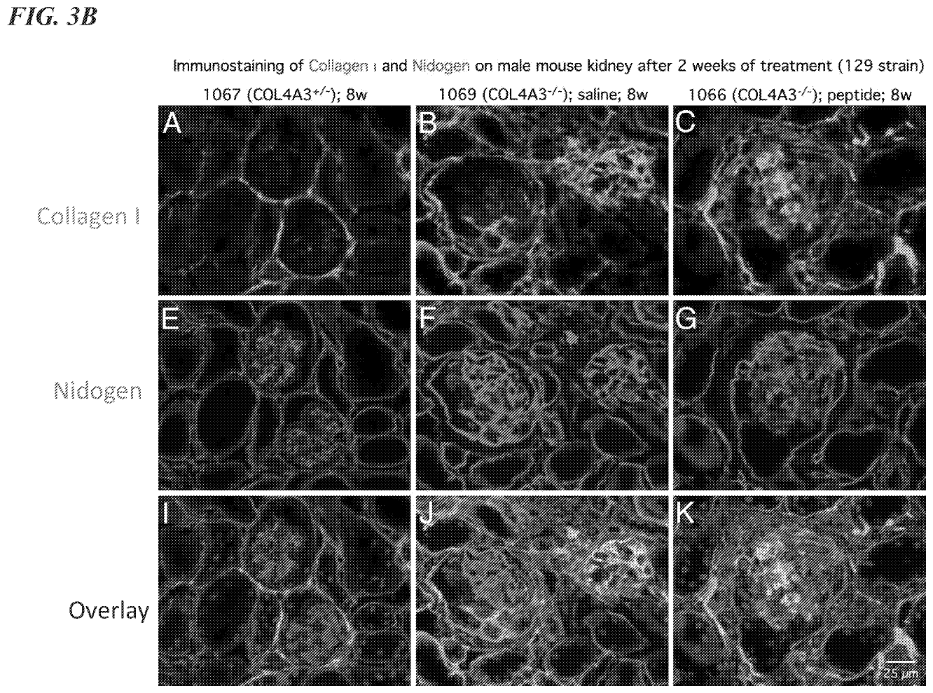

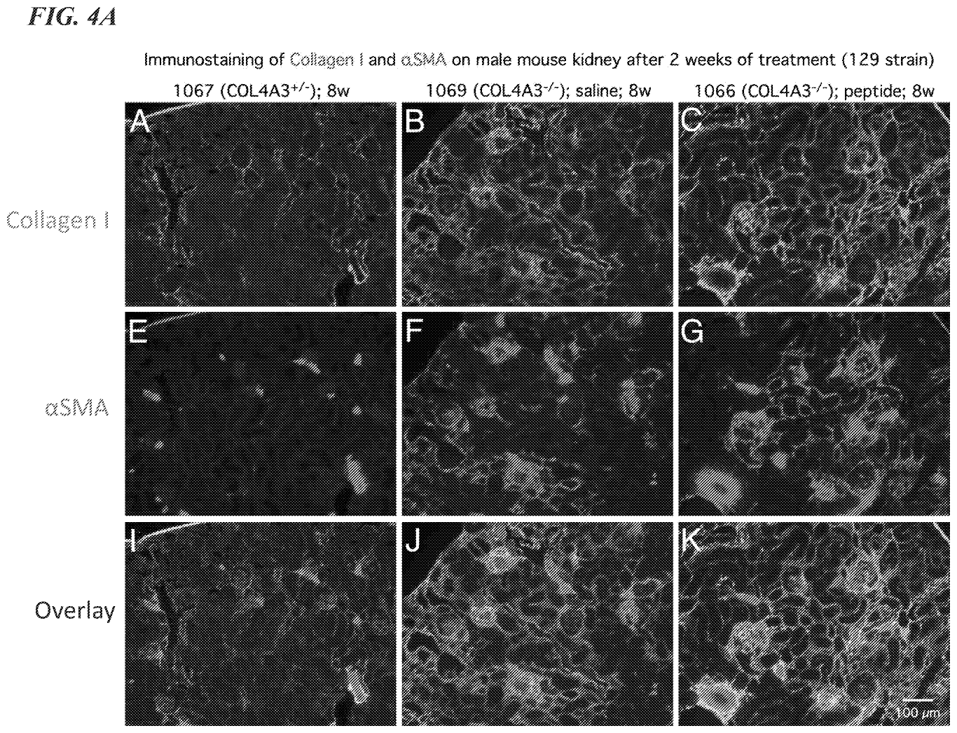

FIG. 3A and FIG. 3B show immunofluorescence staining of collagen I and nidogen on kidney tissue from an untreated Col4a3+/− mouse or Cohort 1 Col4a3−/− mice following 2 weeks of treatment with saline or APi2355 peptide. The Col4a3+/− mouse showed collagen I staining in the interstitium and Bowman's capsule and the saline- and APi2355-treated Col4a3−/− mice showed additional staining in sclerotic glomeruli.

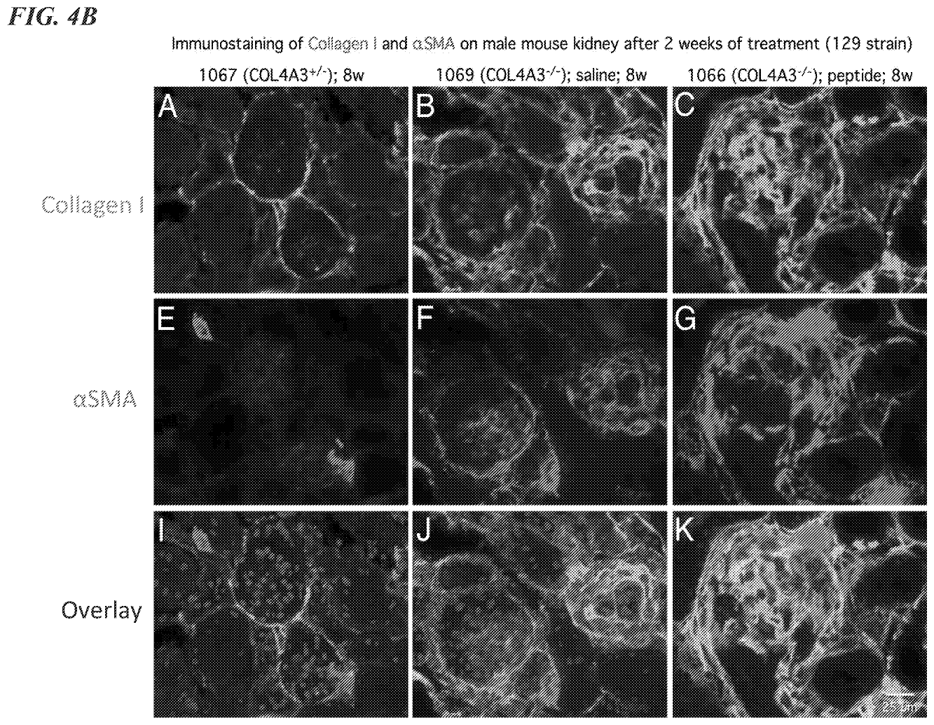

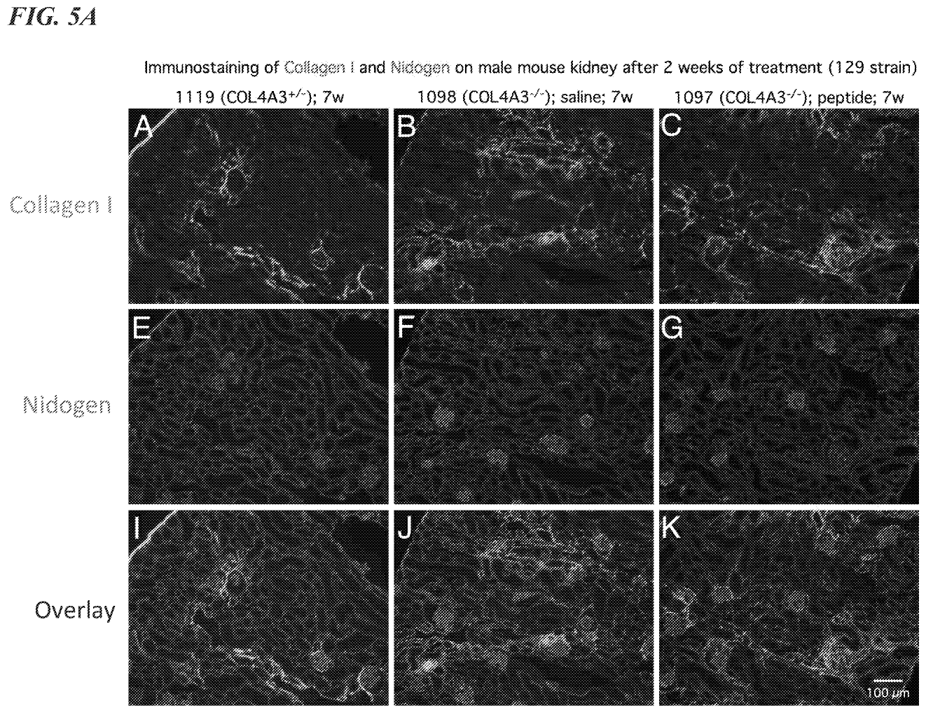

FIG. 4A and FIG. 4B show immunofluorescence staining of collagen I and αSMA on kidney tissue from an untreated Col4a3+/− mouse or Cohort 1 Col4a3−/− mice following 2 weeks of treatment with saline or APi2355 peptide. The Col4a3+/− mouse showed αSMA staining in interstitial blood vessels and the saline- and APi2355-treated Col4a3−/− mice showed additional staining in sclerotic glomeruli.

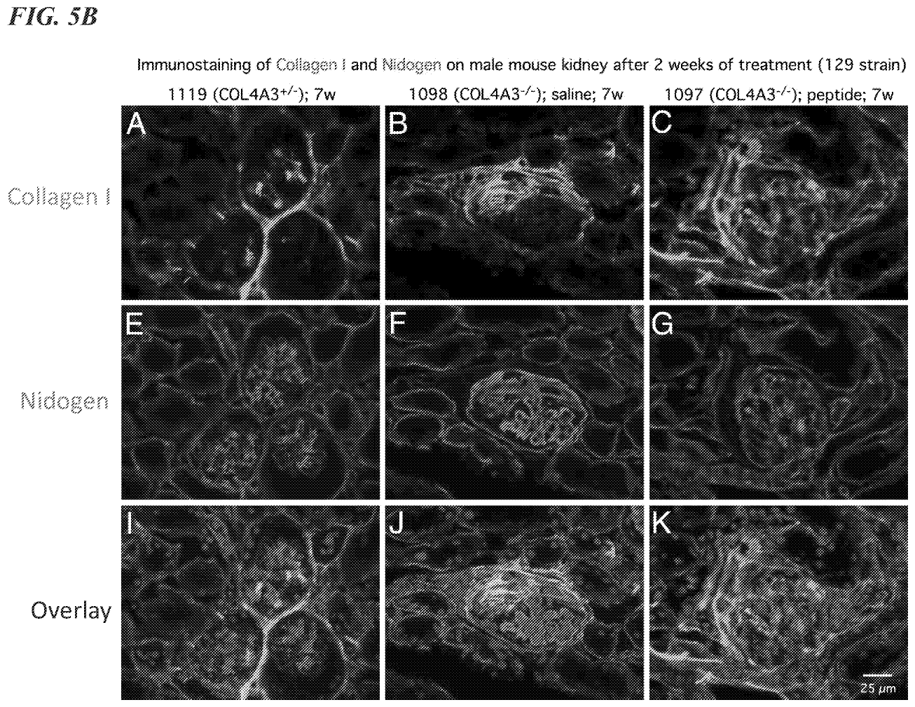

FIG. 5A and FIG. 5B show immunofluorescence staining of collagen I and nidogen on kidney tissue from an untreated Col4a3+/− mouse or Cohort 2 Col4a3−/− mice following 2 weeks of treatment with saline or APi2355 peptide. The Col4a3+/− mouse showed collagen I staining in the interstitium and Bowman's capsule and the saline- and APi2355-treated Col4a3−/− mice showed additional staining in sclerotic glomeruli.

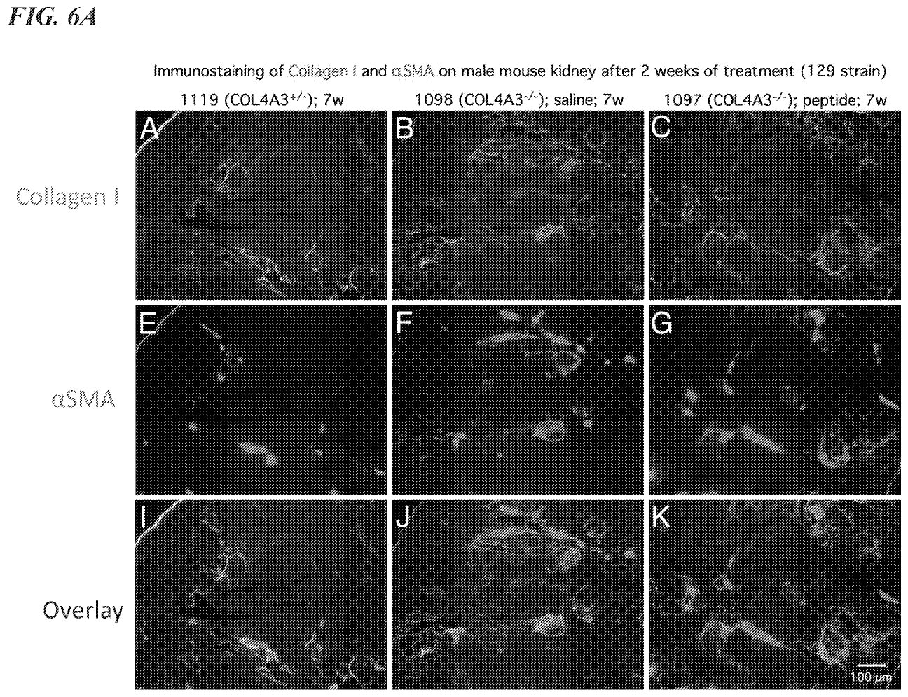

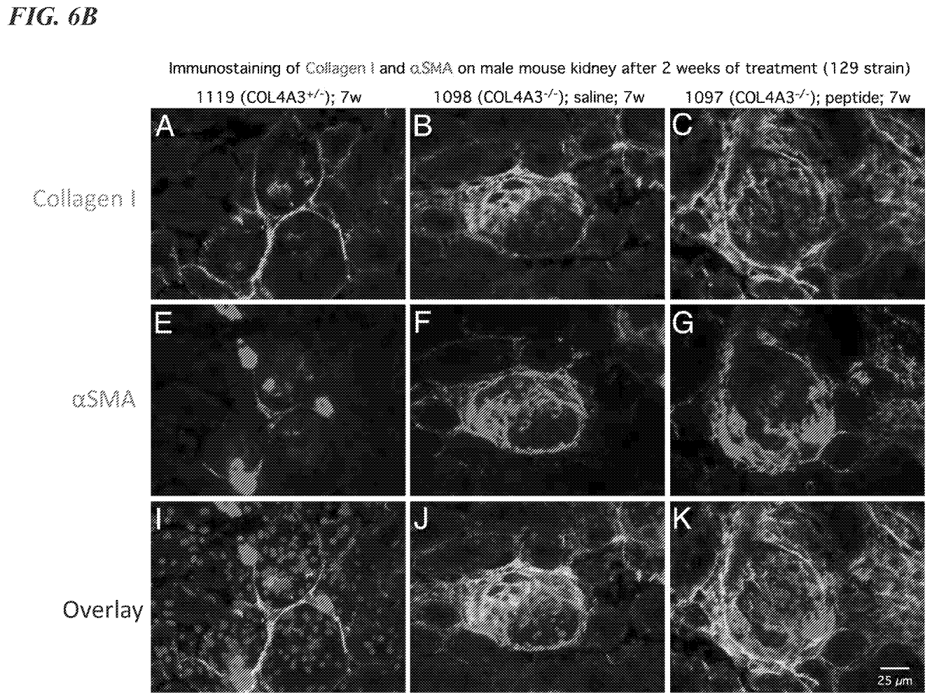

FIG. 6A and FIG. 6B show immunofluorescence staining of collagen I and αSMA on kidney tissue from an untreated Col4a3+/− mouse or Cohort 2 Col4a3−/− mice following 2 weeks of treatment with saline or APi2355 peptide. The Col4a3+/− mouse showed αSMA staining in interstitial blood vessels and the saline- and APi2355-treated Col4a3−/− mice showed additional staining in sclerotic glomeruli.

Overall, both saline- and APi2355-treated Col4a3−/− mice in Cohort 1 and Cohort 2 showed severe sclerosis in the interstitium and many sclerotic glomeruli.

Study 2: Four Weeks of Treatment with Saline or APi2355 in Col4a3−/− Mouse Model of Alport Syndrome

Additional studies were performed to assess the efficacy of modified Cav-1 peptides in ameliorating kidney fibrosis and disease using the Col4a3−/− mouse model of Alport syndrome.

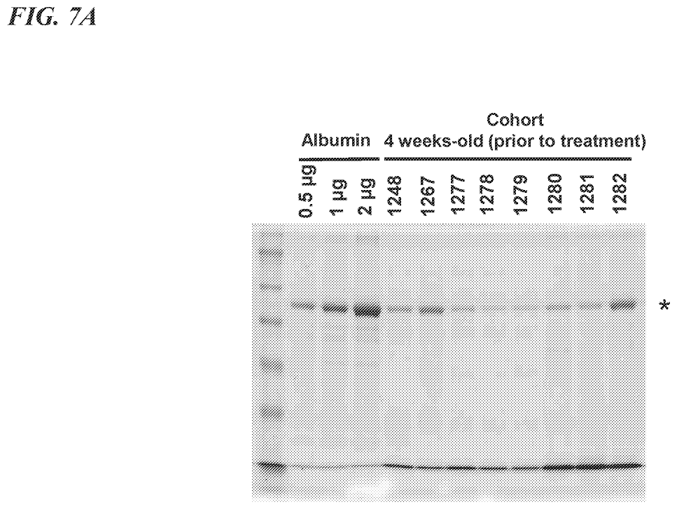

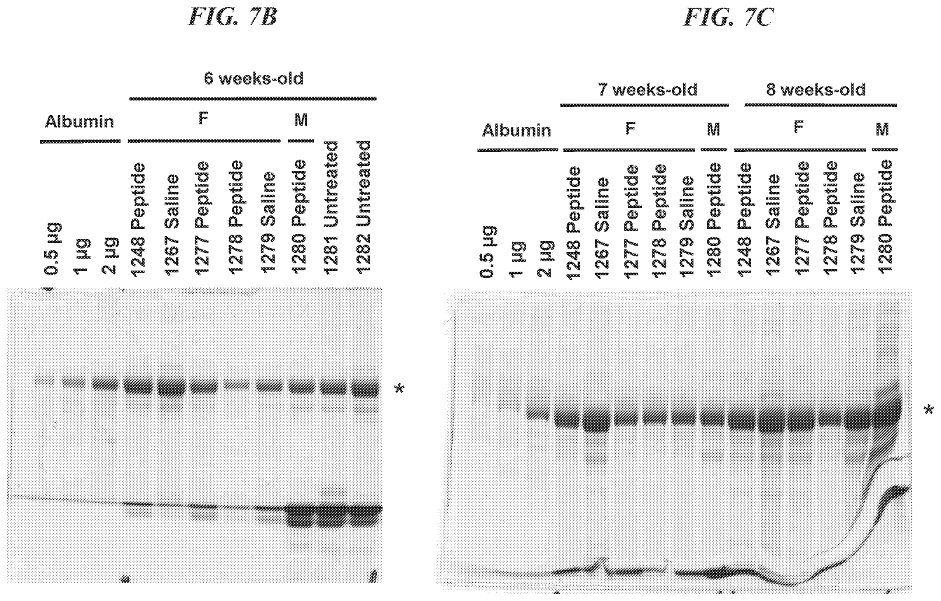

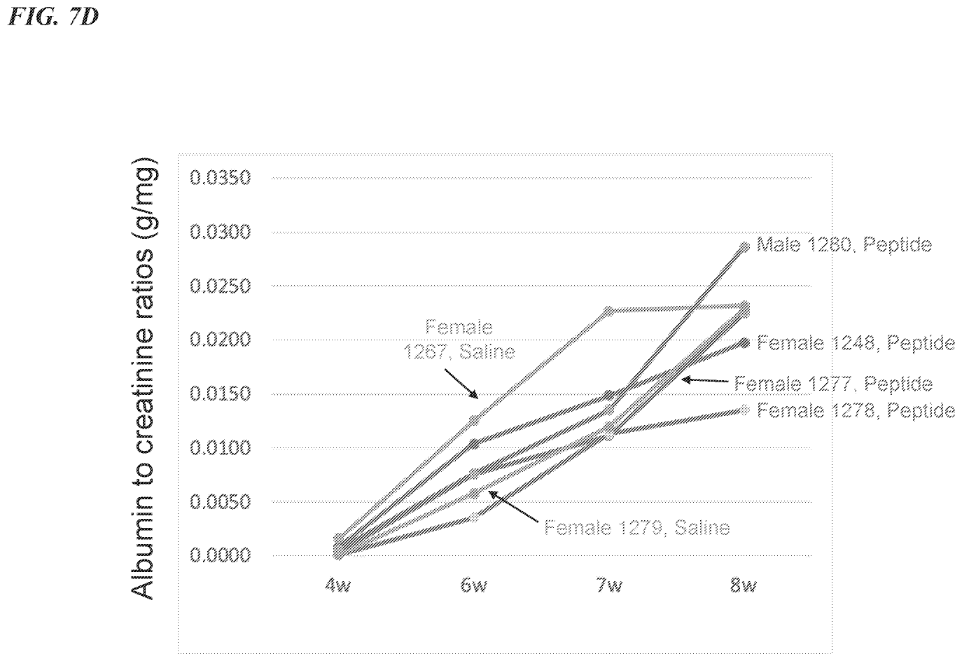

Four week-old Col4a3−/− mice on a 129S1/SvlmJ background were intraperitoneally injected daily with APi2355 (SEQ ID NO: 8, 0.0022 mg/g body weight) or with normal saline for 4 weeks. Urine was collected prior to treatment at 4 weeks-old and at 6, 7, and 8-weeks-old following treatment to monitor for glomerular permselectivity by measuring albumin to creatinine (A/C) ratios. Blood was collected prior to treatment at 4 weeks-old and at 6 and 8 weeks-old following treatment to monitor for glomerular filtration rate using the BUN assay and kidney tissue was collected following sacrifice to assess for renal injury and fibrosis. Mouse identification numbers were used to differentiate treatment groups. (e.g., mice identification nos. 1248, 1267 and 1277-1282 as shown in FIG. 7A).

FIG. 7A shows a urinary protein gel from 4 week-old Col4a3−/− mice prior to treatment with saline or APi2355 peptide. FIG. 7B shows a urinary protein gel from 6 week-old Col4a3−/− mice after 2 weeks of daily intraperitoneal injections with APi2355 peptide or normal saline and FIG. 7C shows a urinary protein gel from 7 and 8 week-old Col4a3−/− mice after 3 or 4 weeks of daily intraperitoneal injections with APi2355 peptide or saline. FIG. 7D shows albumin to creatinine ratios (g/mg) in the urine of Col4a3−/− mice before treatment at 4 weeks-old and during treatment with saline or APi2355 peptide at 6, 7, and 8 weeks-old.

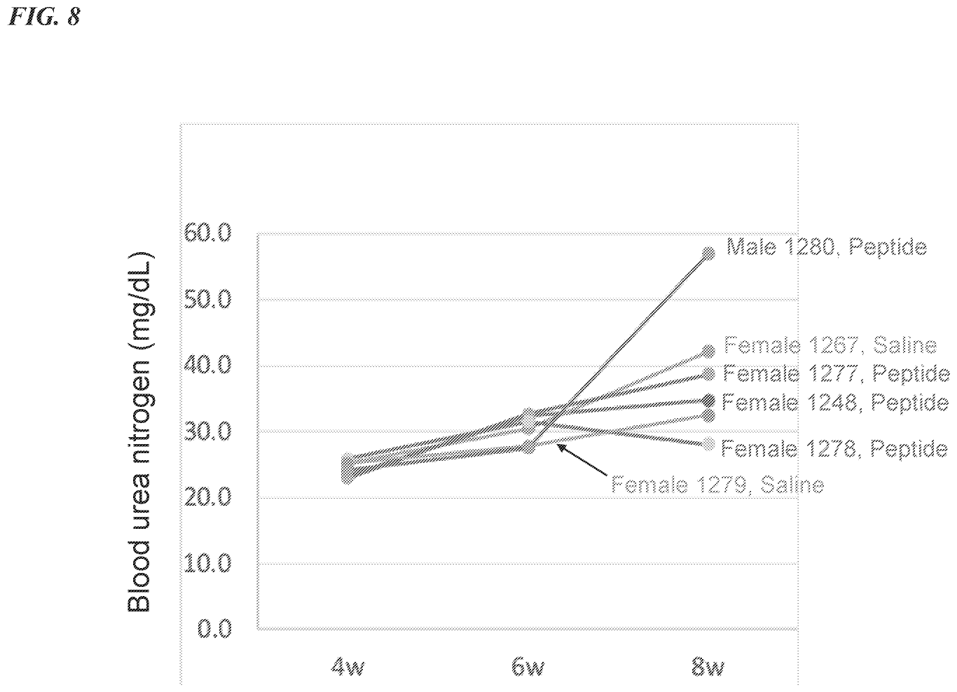

FIG. 8 shows urea nitrogen levels in the blood of Col4a3−/− mice before treatment at 4 weeks-old and during treatment with saline or APi2355 peptide at 6 and 8 weeks-old.

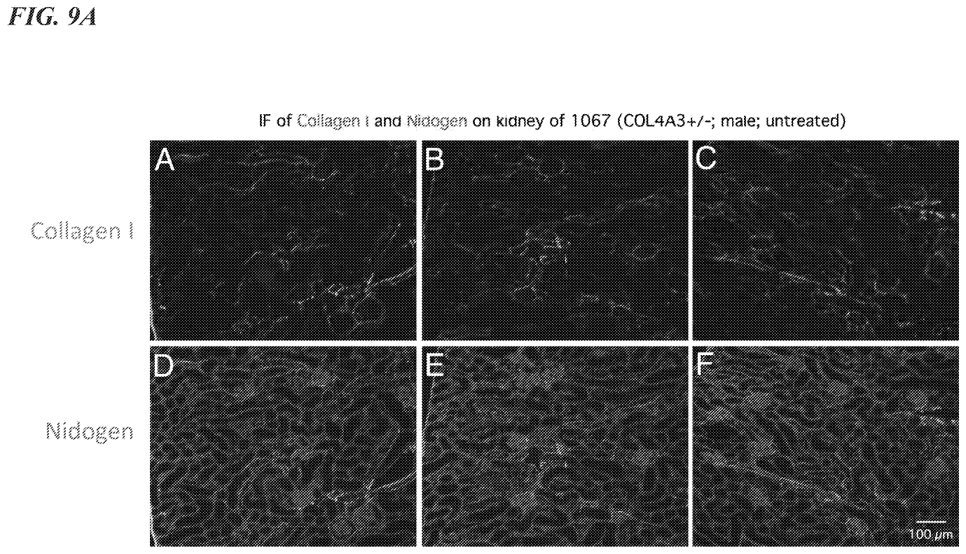

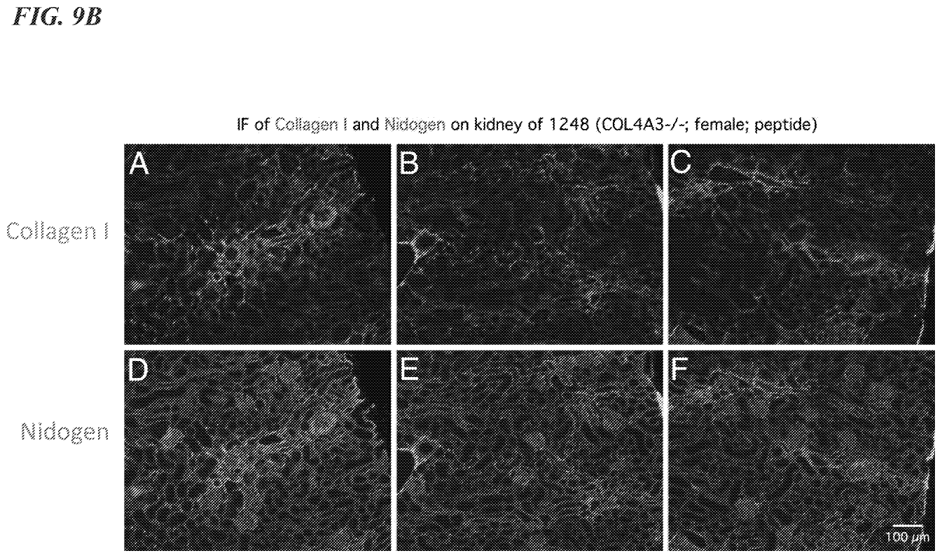

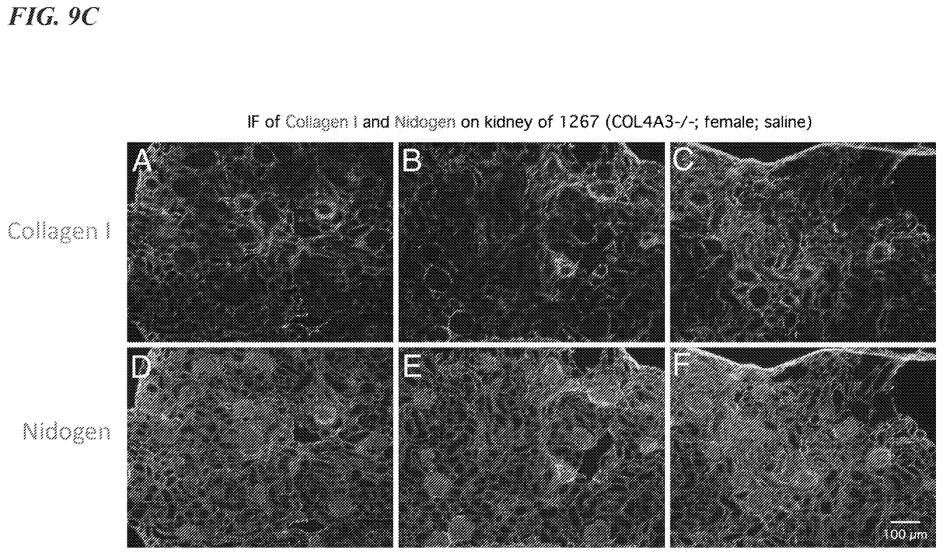

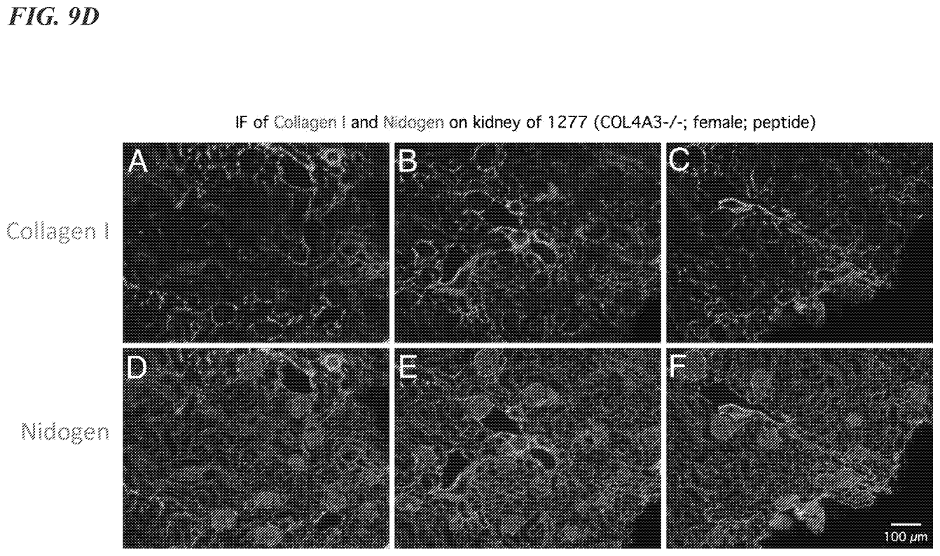

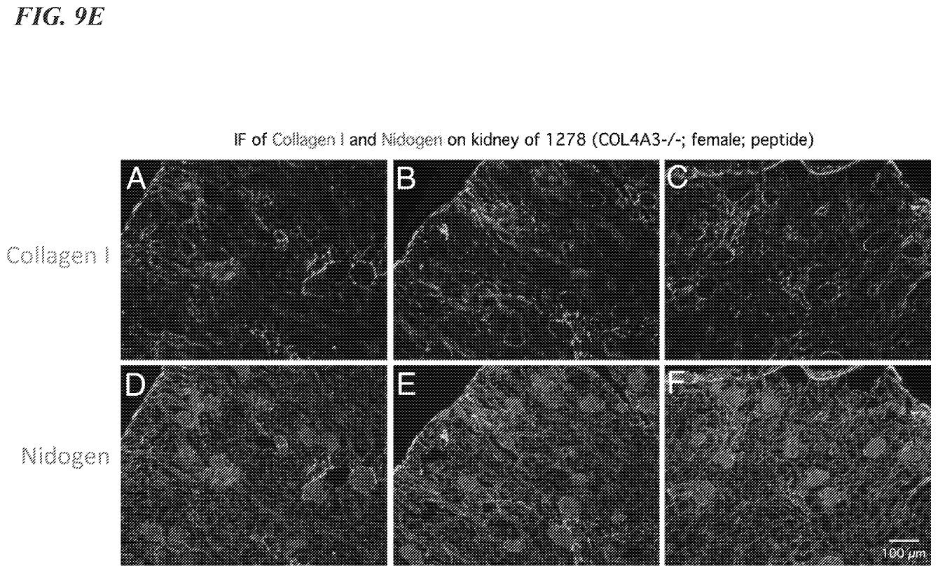

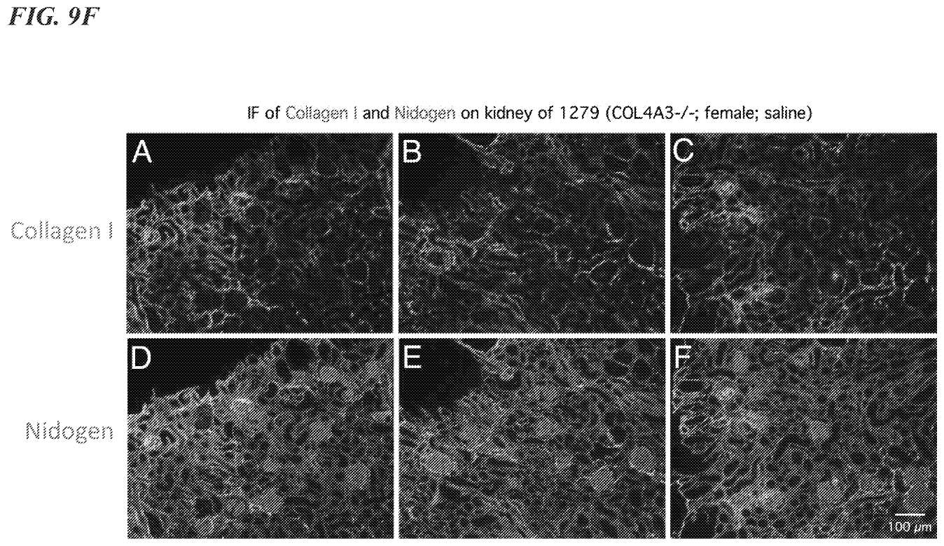

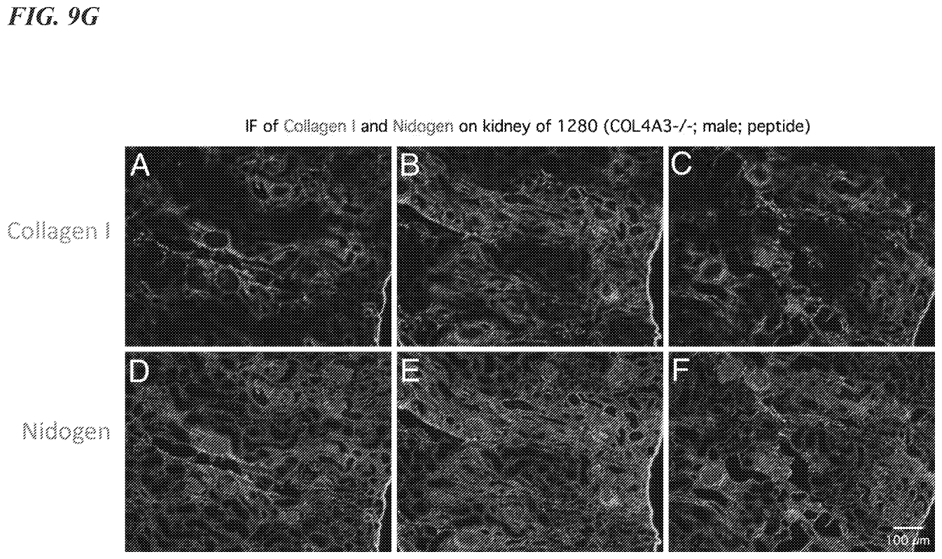

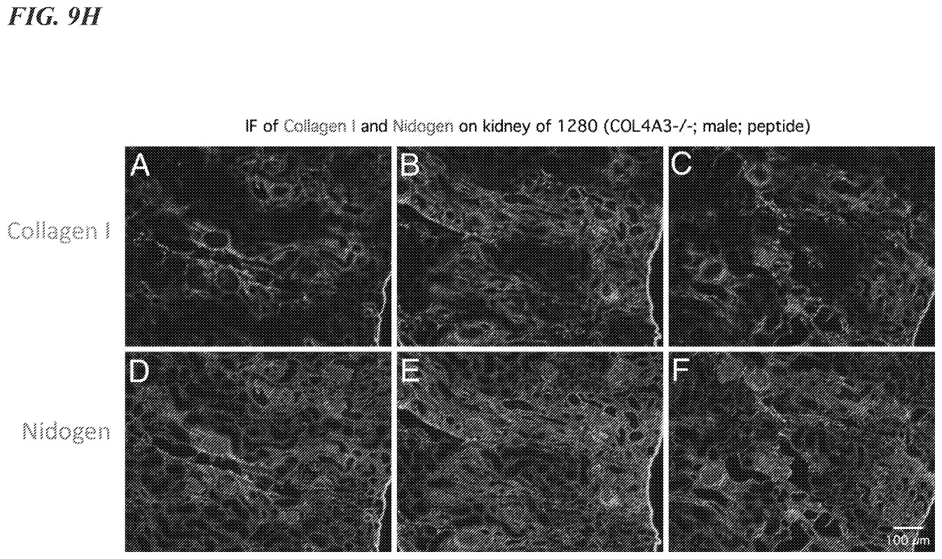

FIG. 9A to FIG. 9H shows immunofluorescence staining of collagen I and nidogen on kidney tissue from an untreated Col4a3+/− mouse or Col4a3−/− mice following 4 weeks of treatment with saline or APi2355 peptide.

Table A shows quantification of glomerular fibrosis in an 8 week-old untreated Col4a3+/− mouse or Col4a3−/− mice following 4 weeks of treatment with normal saline or APi2355 peptide.

TABLE A

| | | | | | Severely | Moderately | Severely | Moderately | Sclerotic |
|---|---|---|---|---|---|---|---|---|---|
| Mouse | | | | Total | Sclerotic | Sclerotic | Sclerotic | Sclerotic | Glom |
| ID | Gender | Genotype | Treatment | Glom | Glom | Glom | Glom (%) | Glom (%) | (%) |
| 1067-3 | Male | Col4a3+/− | None | 35 | 0 | 0 | 0.0 | 0.0 | 0.0 |
| 1248-3 | Female | Col4a3−/− | APi2355 | 51 | 4 | 1 | 7.8 | 2.0 | 9.8 |
| 1267-5 | Female | Col4a3−/− | Saline | 53 | 13 | 4 | 24.5 | 7.5 | 32.1 |
| 1277-5 | Female | Col4a3−/− | APi2355 | 47 | 4 | 1 | 8.5 | 2.1 | 10.6 |
| 1278-6 | Female | Col4a3−/− | APi2355 | 54 | 4 | 0 | 7.4 | 0.0 | 7.4 |
| 1279-7 | Female | Col4a3−/− | Saline | 49 | 8 | 3 | 16.3 | 6.1 | 22.4 |
| 1280-1 | Male | Col4a3−/− | APi2355 | 40 | 21 | 6 | 52.5 | 15.0 | 67.5 |

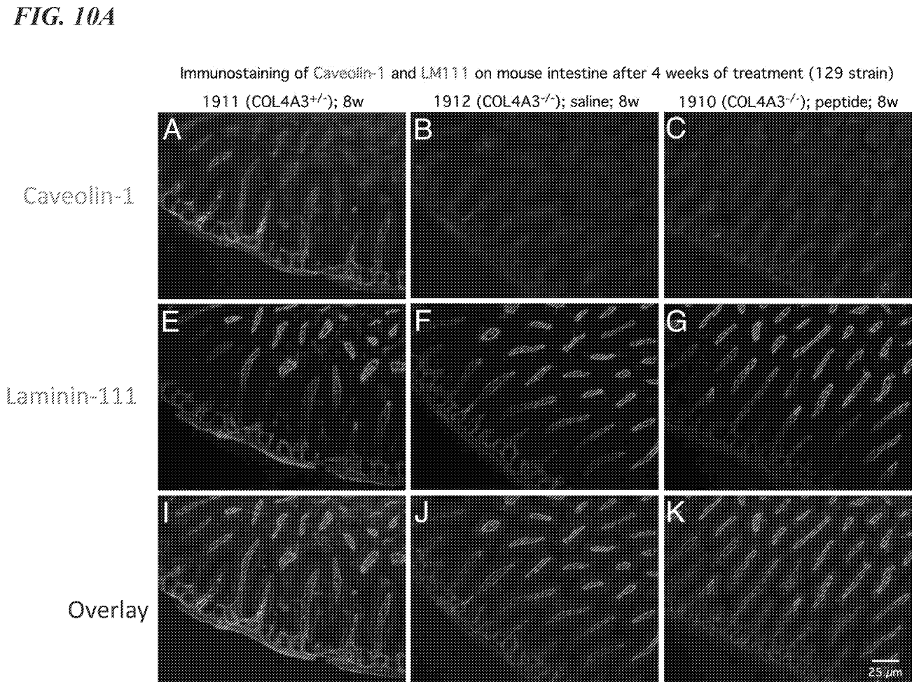

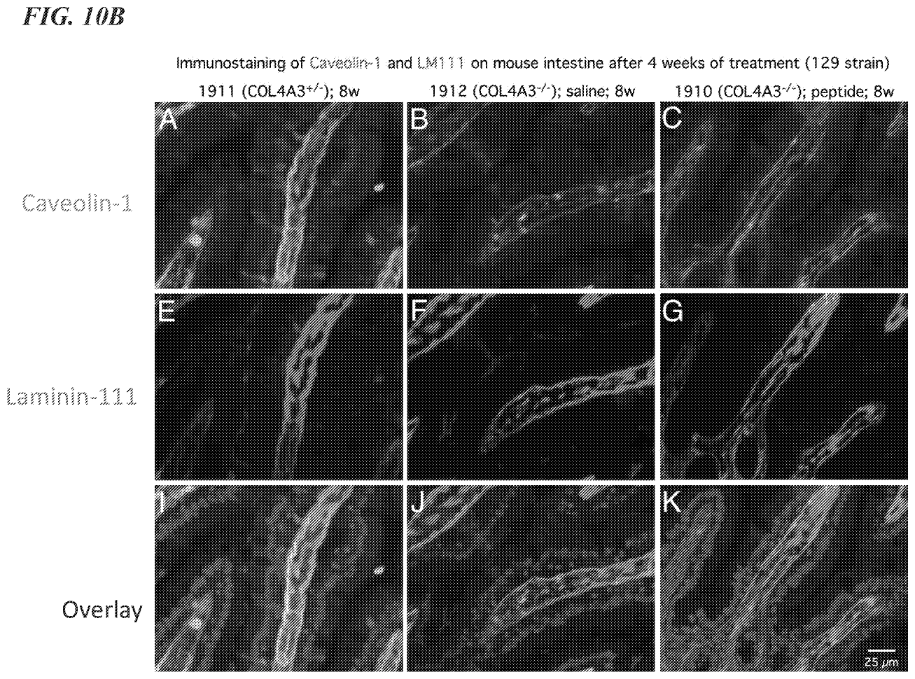

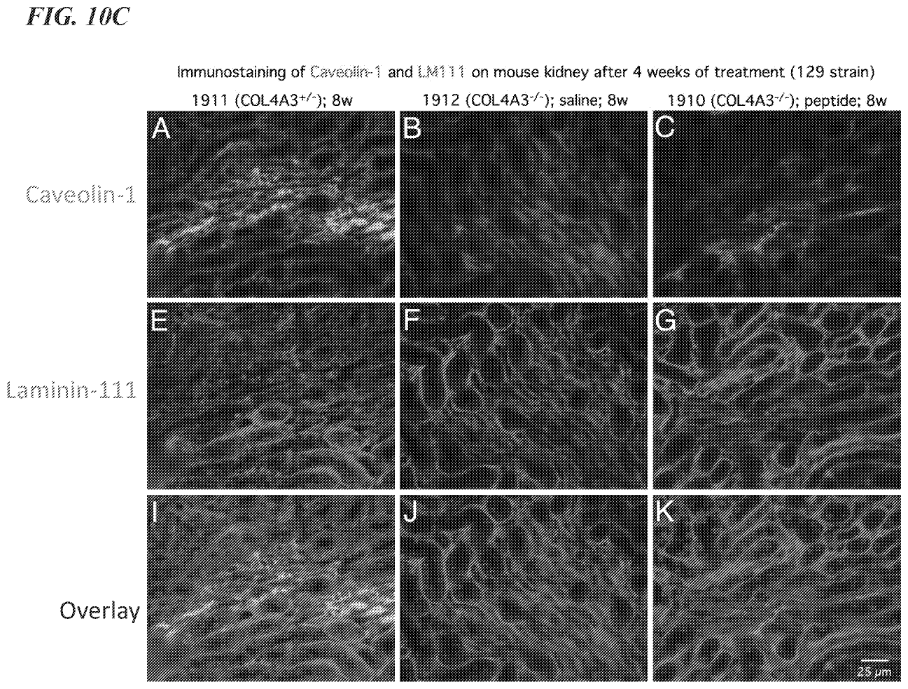

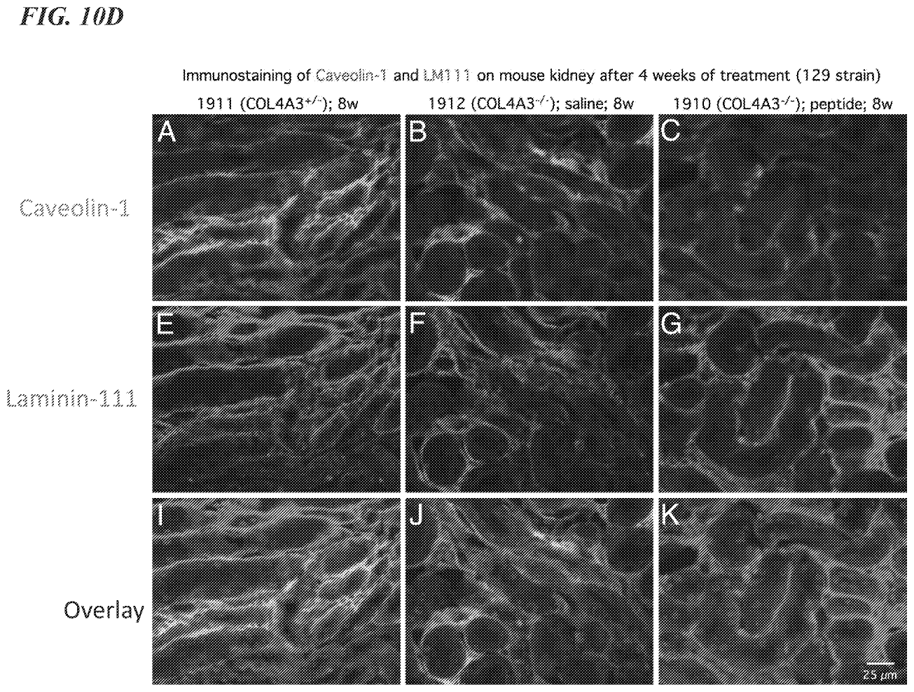

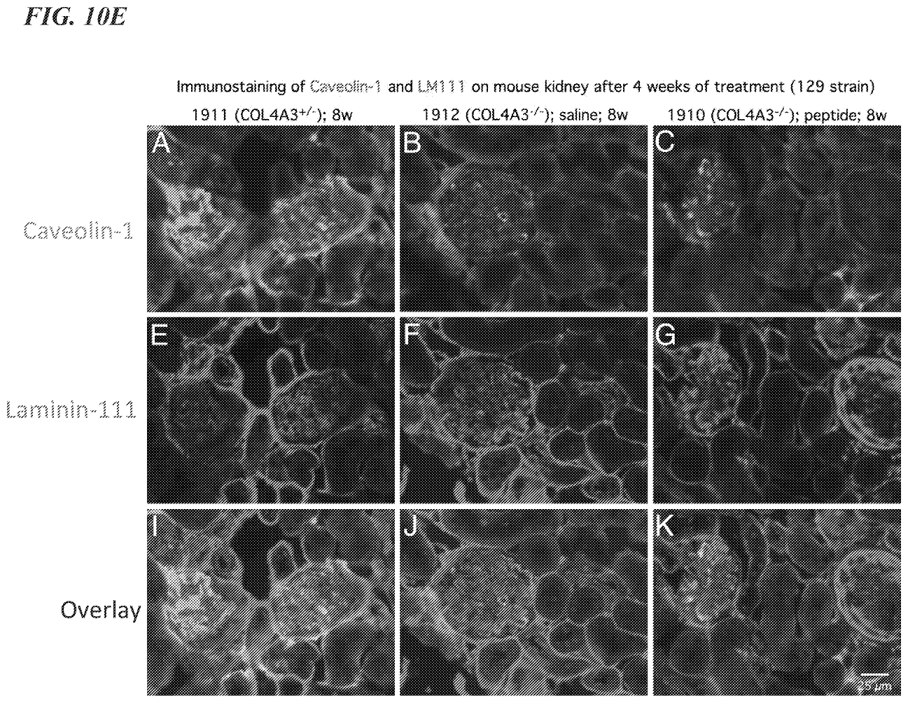

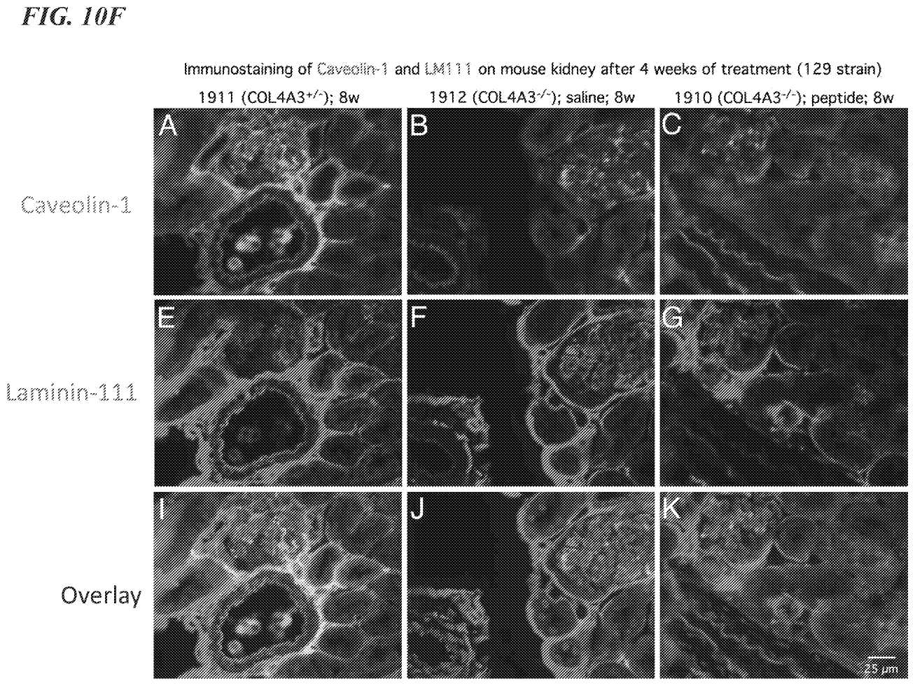

FIG. 10A and FIG. 10B show immunofluorescence staining of caveolin-1 and laminin-111 on intestine tissue from an untreated Col4a3+/− mouse (left panel) or Col4a3−/− mice following 4 weeks of treatment with saline (middle panel) or APi2355 peptide (right panel). FIG. 10C to FIG. 10F shows immunofluorescence staining of caveolin-1 and laminin-111 on kidney tissue from an untreated Col4a3+/− mouse (left panel) or Col4a3−/− mice following 4 weeks of treatment with saline (middle panel) or APi2355 peptide (right panel).

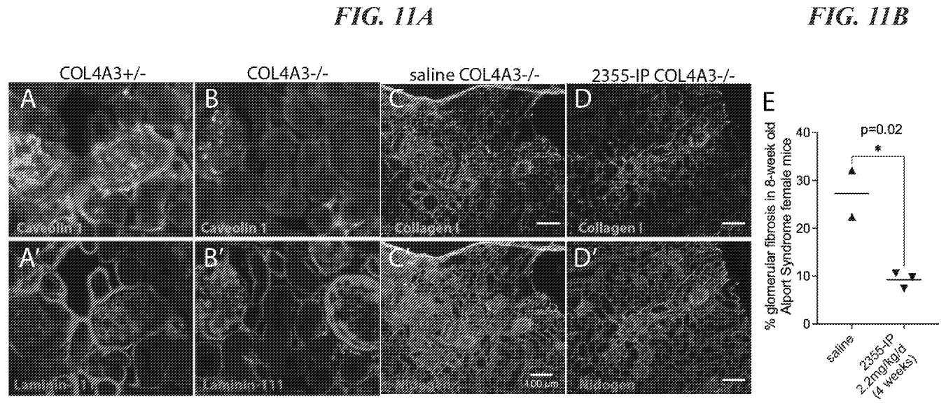

FIG. 11A shows immunofluorescence staining of caveolin-1, collagen I, laminin-111, and nidogen on kidney tissue from untreated Col4a3+/− and Col4a3−/− mice or Col4a3−/− mice following 4 weeks of treatment with saline or APi2355 peptide. FIG. 11B shows quantification of glomerular fibrosis in 8 week-old female Col4a3−/− mice following 4 weeks of treatment with normal saline or APi2355 peptide.

Overall, treatment of Col4a+/− mice with APi2355 peptide starting at 4 weeks of age did not slow the progression of albuminuria and BUN but reduced the percentage of sclerotic glomeruli in females as assayed by collagen I deposition. These data indicate that APi2355 reduced glomerular fibrosis in a mouse model of Alport syndrome.

### Example 2: Efficacy of Modified Cav-1 Peptides in an Aged Mouse Model of Lung Fibrosis

The objective of this study was to evaluate the modified Cav-1 peptide CSP-7 in a bleomycin model of idiopathic pulmonary fibrosis and acute respiratory distress syndrome (ARDS). Aged male mice were selected to closely match the population of humans most vulnerable to ARDS.

### Study 1

Aged male mice, which have a higher burden of senescent cells and therefore less regenerative potential, were used to test the efficacy of CSP-7 (SEQ ID NO: 3). Mice were between 74 to 76 weeks-old at the time of intratracheal bleomycin installation (4 U/kg body weight; n=5-8 mice/group). The Total Collagen Assay (QuickZyme Biosciences) was used to assess total collagen in lung tissue, which first hydrolyzes proteins in the lung homogenate then oxidizes hydroxyproline and stains.

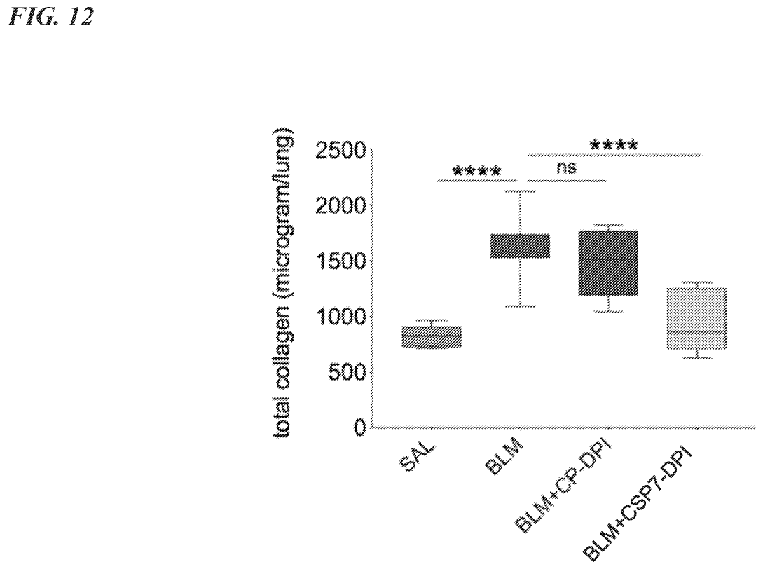

FIG. 12 shows total collagen in lung homogenates of saline or bleomycin-treated aged mice administered control peptide (CP-DPI) or CSP-7 (CSP7-DPI) through dry powder inhalation. Bleomycin-treated mice administered CSP-7 showed a reduction in total collagen in the lung compared to mice administered control peptide.

### Study 2

Bleomycin (BLM, 8 U/kg) was administered intratracheally to 73-74 week-old mice (n=52) expecting approximately 50% loss of mice due to BLM-induced lung injury. On day 14 post-instillation, CSP-7 and a control peptide (CP) was administered to mice via dry powder inhalation (DPI) or intraperitoneal injection (IP) as shown in Table B below. On day 21 post-instillation, mice were sacrificed and blood and tissues were collected for analysis. Statistics were performed using Dunnett's multiple comparison test.

TABLE B

| | | BLM Dose | | | | |
|---|---|---|---|---|---|---|
| Group | Description | (U/kg) | Treatment | Dose | Route | n |
| A | BLM CSP-7 DPI | 8 | CSP-7 | 0.5 mg/mouse | DPI | 8 |
| D | BLM CP DPI | 8 | Control Peptide | 0.5 mg/mouse | DPI | 8 |
| E | BLM CSP-7 IP | 8 | CSP-7 | 1.5 mg/kg | IP | 6 |
| B | BLM control | 8 | None | None | — | 8 |
| C | Saline control | Saline | None | None | — | 8 |
| | | control | | | | |

| | |
|---|---|
| Total Animals | 38 |

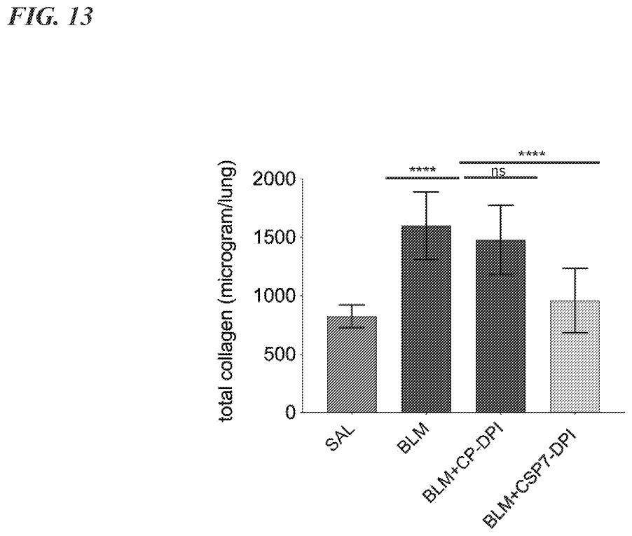

FIG. 13 shows collagen in total lung homogenates of saline or BLM-treated aged mice administered control peptide (CP-DPI) or CSP-7 (CSP7-DPI) through dry powder inhalation. BLM-treated mice administered CSP-7 showed a reduction in total collagen in the lung compared to mice administered control peptide.

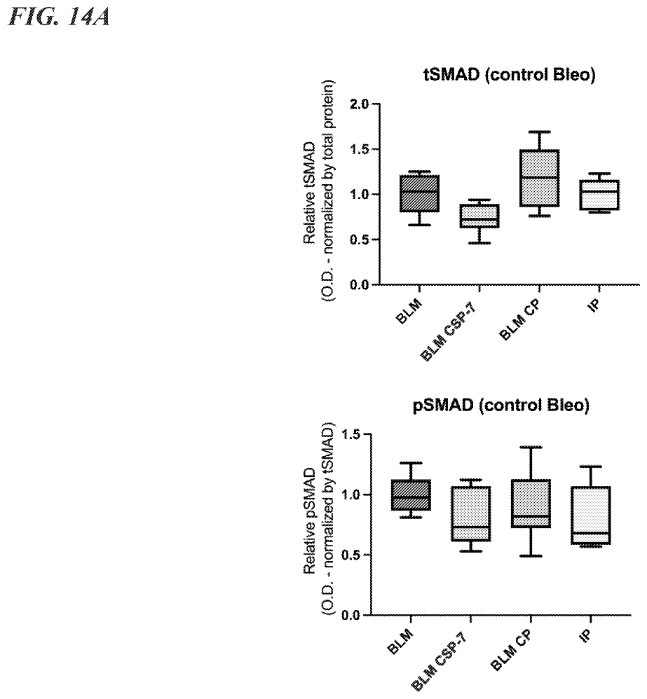

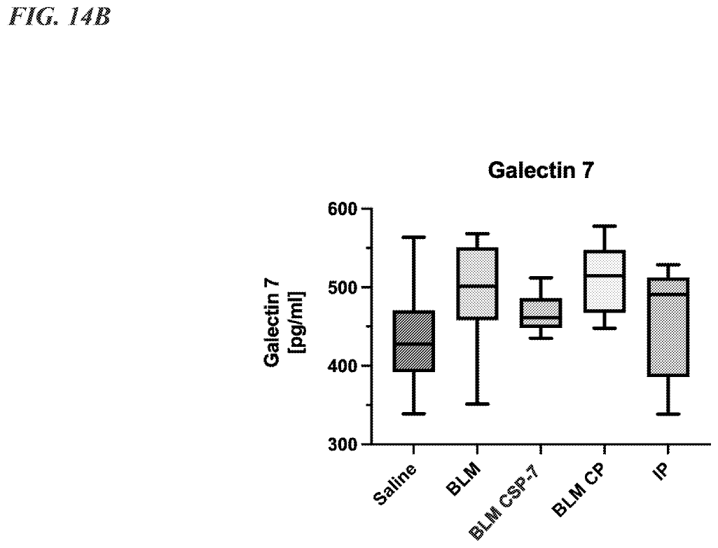

FIG. 14A shows total SMAD (tSMAD, top panel) and phosphorylated SMAD (pSMAD, bottom panel) in total lung homogenates of BLM-treated aged mice administered control peptide (CP-DPI) or CSP-7 (CSP7-DPI) through dry powder inhalation or CSP-7 through intraperitoneal injection (IP).

FIG. 14B shows galectin-7 in total lung homogenates of BLM-treated aged mice administered control peptide (CP-DPI) or CSP-7 (CSP7-DPI) through dry powder inhalation or CSP-7 through intraperitoneal injection (IP).

### Example 3: Human Caveolin-1 Expression in Normal and Fibrotic Kidney Tissue

The objective of this study was to evaluate the expression of human caveolin-1 in kidney tissue from normal subjects and subjects with fibrotic kidney disease.

Kidney tissue was harvested from normal subjects and subjects with fibrotic kidney disease. The kidney tissue was then processed and immunostained for human caveolin-1.

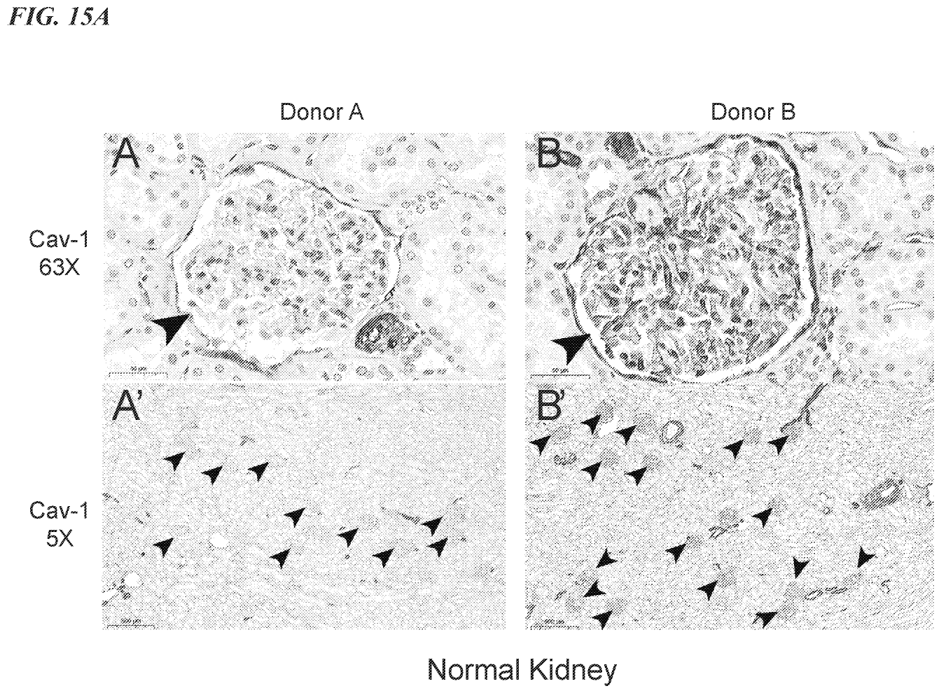

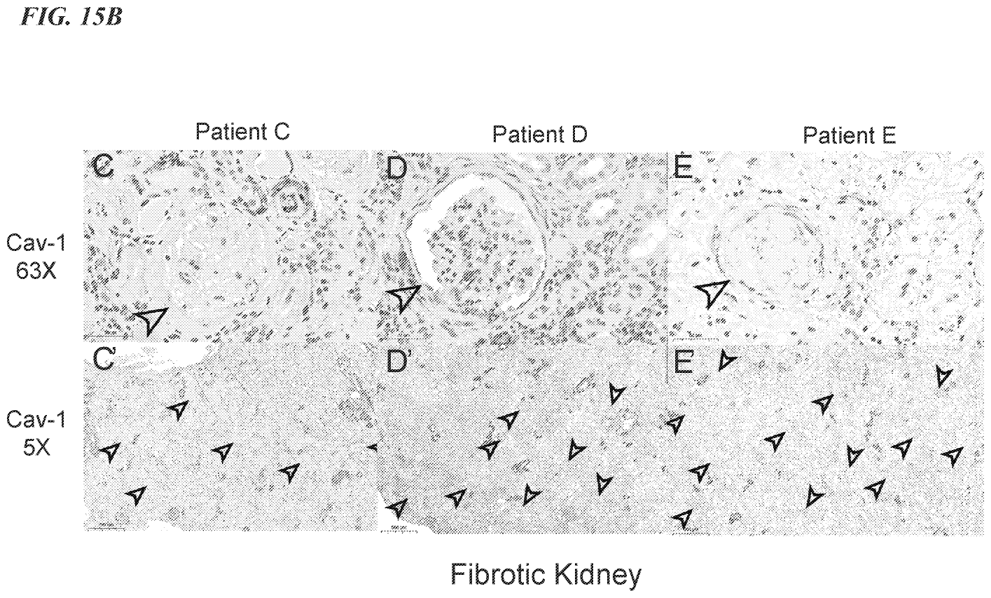

As shown in FIG. 15A, human caveolin-1 staining was detected in the glomerulus of kidney tissue from normal subjects. Specifically, human caveolin-1 was detected in parietal epithelial cells lining the Bowman's capsule as well as endothelial cells of the inner capsule. As shown in FIG. 15B, caveolin-1 expression was reduced in fibrotic glomeruli in subjects with chronic kidney disease compared to normal controls. Histology also indicated significant endothelial destruction in addition to fibrotic glomeruli.

### NUMBERED EMBODIMENTS OF THE INVENTION

Notwithstanding the appended claims, the disclosure sets forth the following numbered embodiments:

Embodiment 1. A method of treating or preventing a kidney disease or disorder in a subject comprising administering to the subject an effective amount of a modified Cav-1 peptide:
(a) consisting of any one of the amino acid sequences of SEQ ID NOs: 2-111;
(b) comprising any one of the amino acid sequences of SEQ ID NOs: 2-111; or
(c) comprising any one of the amino acid sequences of SEQ ID NOs: 2-111 with one or more amino acid substitutions, insertions, deletions, or chemical modifications.

Embodiment 2. The method of embodiment 1, wherein the modified Cav-1 peptide comprises L-amino acids.

Embodiment 3. The method of embodiment 1, wherein the modified Cav-1 peptide comprises D-amino acids.

Embodiment 4. The method of embodiment 1, wherein the modified Cav-1 peptide comprises both L- and D-amino acids.

Embodiment 5. The method of any one of embodiments 1-4, wherein the modified Cav-1 peptide comprises deuterated residues.

Embodiment 6. The method of any one of embodiments 1-5, wherein the modified Cav-1 peptide comprises at least one non-standard amino acid.

Embodiment 7. The method of embodiment 6, wherein the non-standard amino acid is ornithine.

Embodiment 8. The method of any one of embodiments 1-7, wherein the modified Cav-1 peptide comprises an N-terminal modification.

Embodiment 9. The method of any one of embodiments 1-7, wherein the modified Cav-1 peptide comprises a C-terminal modification.

Embodiment 10. The method of any one of embodiments 1-7, wherein the modified Cav-1 peptide comprises an N-terminal modification and a C-terminal modification.

Embodiment 11. The method of embodiment 8 or 10, wherein the N-terminal modification is acylation.

Embodiment 12. The method of embodiment 9 or 10, wherein the C-terminal modification is amidation.

Embodiment 13. The method of embodiment 1, wherein the modified Cav-1 peptide comprises the amino acid sequence of FTTFTVT (SEQ ID NO: 3).

Embodiment 14. The method of embodiment 1, wherein the modified Cav-1 peptide comprises the amino acid sequence of KASFTTFTVTKGS (SEQ ID NO: 4).

Embodiment 15. The method of embodiment 1, wherein the modified Cav-1 peptide comprises the amino acid sequence of KASFTTFTVTKGS-NH2 (SEQ ID NO: 5).

Embodiment 16. The method of embodiment 1, wherein the modified Cav-1 peptide comprises the amino acid sequence of aaEGKASFTTFTVTKGSaa (SEQ ID NO: 6).

Embodiment 17. The method of embodiment 1, wherein the modified Cav-1 peptide comprises the amino acid sequence of aaEGKASFTTFTVTKGSaa-NH2 (SEQ ID NO: 7).

Embodiment 18. The method of embodiment 1, wherein the modified Cav-1 peptide comprises the amino acid sequence of Ac-aaEGKASFTTFTVTKGSaa-NH2 (SEQ ID NO: 8).

Embodiment 19. The method of embodiment 1, wherein the modified Cav-1 peptide comprises the amino acid sequence of OASFTTFTVTOS (SEQ ID NO: 9).

Embodiment 20. The method of embodiment 1, wherein the modified Cav-1 peptide comprises the amino acid sequence of OASFTTFTVTOS-NH2 (SEQ ID NO: 10).

Embodiment 21. The method of any one of embodiments 1-20, wherein the modified Cav-1 peptide comprises an internalization sequence.

Embodiment 22. The method of embodiment 21, wherein the internalization sequence is located at the C-terminal end of the peptide.

Embodiment 23. The method of embodiment 21, wherein the internalization sequence is located at the N-terminal end of the peptide.

Embodiment 24. The method of any one of embodiments 1-23, wherein the modified Cav-1 peptide further comprises a cap at the N- and/or C-terminus.

Embodiment 25. The method of embodiment 24, wherein the modified Cav-1 peptide comprises the cap at the N-terminus and C-terminus.

Embodiment 26. The method of any one of embodiments 1-25, wherein the modified Cav-1 peptide is cyclized.

Embodiment 27. The method of any one of embodiments 1-26, wherein the modified Cav-1 peptide maintains the biological activity of native Cav-1 (SEQ ID NO: 1).

Embodiment 28. The method of embodiment 1, wherein the modified Cav-1 peptide is a multimer comprising at least two peptides of any one of embodiments 1-25.

Embodiment 29. The method of embodiment 28, wherein a first peptide of the at least two peptides is essentially identical to a second peptide of the at least two peptides.

Embodiment 30. The method of embodiment 28, wherein a first peptide of the at least two peptides is not identical to a second peptide of the at least two peptides.

Embodiment 31. The method of any one of embodiments 1-30, wherein the modified Cav-1 peptide is administered intravenously, subcutaneously, intramuscularly, intraperitoneally, or orally.

Embodiment 32. The method of embodiment 31, wherein the modified Cav-1 peptide is administered subcutaneously.

Embodiment 33. The method of embodiment 31, wherein the modified Cav-1 peptide is administered intravenously.

Embodiment 34. The method of any one of embodiments 1-33, wherein the kidney disease or disorder is selected from the group consisting of chronic kidney disease, end-stage renal disease, glomerulonephritis, focal segmental glomerulosclerosis, kidney fibrosis, polycystic kidney disease, IgA nephropathy, lupus nephritis, nephrotic syndrome, Alport syndrome, amyloidosis, Goodpasture syndrome, granulomatosis with polyangiitis, or acute kidney injury.

Embodiment 35. The method of embodiment 34, wherein the kidney disease or disorder is Alport syndrome.

Embodiment 36. The method of any one of embodiments 1-33, wherein the kidney disease or disorder is characterized by fibrosis.

Embodiment 37. The method of any one of embodiments 1-36, wherein the method further comprises administering an effective amount of at least one additional therapeutic agent.

Embodiment 38. The method of embodiment 37, wherein the at least one additional therapeutic agent is an angiotensin-converting enzyme (ACE) inhibitor and/or angiotensin II receptor (ARB) inhibitor.

Embodiment 39. The method of any one of embodiments 1-38, wherein the method further comprises treating the subject with dialysis.

Embodiment 40. The method of any one of embodiments 1-39, wherein the modified Cav-1 peptide is administered as a composition comprising the modified Cav-1 peptide and at least one pharmaceutically acceptable carrier or excipient.

Embodiment 41. A method of treating or preventing a fibrotic disease or disorder in an elderly subject comprising administering to the subject an effective amount of a modified Cav-1 peptide:
(a) consisting of any one of the amino acid sequences of SEQ ID NOs: 2-111;
(b) comprising any one of the amino acid sequences of SEQ ID NOs: 2-111; or
(c) comprising any one of the amino acid sequences of SEQ ID NOs: 2-111 with one or more amino acid substitutions, insertions, deletions, or chemical modifications.

Embodiment 42. The method of embodiment 41, wherein the modified Cav-1 peptide comprises the amino acid sequence of FTTFTVT (SEQ ID NO: 3).

Embodiment 43. The method of embodiment 41, wherein the modified Cav-1 peptide comprises the amino acid sequence of Ac-aaEGKASFTTFTVTKGSaa-NH2 (SEQ ID NO: 8).

Embodiment 44. The method of any one of embodiments 41-43, wherein the fibrotic disease or disorder is interstitial lung disease.

Embodiment 45. The method of embodiment 44, wherein the interstitial lung disease is idiopathic pulmonary fibrosis.

Embodiment 46. The method of any one of embodiments 41-45, wherein the modified Cav-1 peptide is administered to the lung.

Embodiment 47. The method of embodiment 46, wherein the modified Cav-1 peptide is administered to the lung via inhalation.

Embodiment 48. The method of any one of embodiments 41-47, wherein the modified Cav-1 peptide is formulated for inhalation.

Embodiment 49. The method of embodiment 48, wherein the modified Cav-1 peptide is formulated for pressurized metered dose inhalation.

Embodiment 50. The method of embodiment 48, wherein the modified Cav-1 peptide is formulated as a dry powder.

Embodiment 51. The method of embodiment 50, wherein the dry powder comprising the modified Cav-1 peptide is essentially excipient free.

Embodiment 52. The method of embodiment 50 or 51, wherein the dry powder is produced by a spray-drying process, air jet milling, ball milling, or wet milling.

Embodiment 53. The method of embodiment 48, wherein the modified Cav-1 peptide is formulated for nebulization.

Embodiment 54. The method of embodiment 47, wherein the modified Cav-1 peptide is administered to the subject using a nebulizer.

Embodiment 55. The method of embodiment 47, wherein the modified Cav-1 peptide is administered to the subject using an inhaler.

Embodiment 56. The method of any one of embodiments 41-55, wherein the method further comprises administering to the subject a therapeutically effective amount of at least one additional therapeutic agent.

Embodiment 57. The method of embodiment 56, wherein the at least one additional therapeutic agent is chloroquine, hydroxychloroquine, remdesivir, favipiravir, lopinavir, or ritonavir.

Embodiment 58. The method of any one of embodiments 41-57, wherein the subject is at least 55 years old, at least 60 years old, at least 65 years old, at least 70 years old, at least 75 years old, at least 80 years old, at least 85 years old, or at least 90 years old.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. A method of treating or preventing a kidney disease or disorder in a subject comprising administering to the subject an effective amount of a modified Cav-1 peptide:
(d) consisting of any one of the amino acid sequences of SEQ ID NOs: 2-111;
(e) comprising any one of the amino acid sequences of SEQ ID NOs: 2-111; or
(f) comprising any one of the amino acid sequences of SEQ ID NOs: 2-111 with one or more amino acid substitutions, insertions, deletions, or chemical modifications.

2. The method of claim 1, wherein the modified Cav-1 peptide comprises L-amino acids.

3. The method of claim 1, wherein the modified Cav-1 peptide comprises D-amino acids.

4. The method of claim 1, wherein the modified Cav-1 peptide comprises both L- and D-amino acids.

5. The method of any one of claims 1-4, wherein the modified Cav-1 peptide comprises deuterated residues.

6. The method of any one of claims 1-5, wherein the modified Cav-1 peptide comprises at least one non-standard amino acid.

7. The method of claim 6, wherein the non-standard amino acid is ornithine.

8. The method of any one of claims 1-7, wherein the modified Cav-1 peptide comprises an N-terminal modification.

9. The method of any one of claims 1-7, wherein the modified Cav-1 peptide comprises a C-terminal modification.

10. The method of any one of claims 1-7, wherein the modified Cav-1 peptide comprises an N-terminal modification and a C-terminal modification.

11. The method of claim 8 or 10, wherein the N-terminal modification is acylation.

12. The method of claim 9 or 10, wherein the C-terminal modification is amidation.

13. The method of claim 1, wherein the modified Cav-1 peptide comprises the amino acid sequence of FTTFTVT (SEQ ID NO: 3).

14. The method of claim 1, wherein the modified Cav-1 peptide comprises the amino acid sequence of KASFTTFTVTKGS (SEQ ID NO: 4).

15. The method of claim 1, wherein the modified Cav-1 peptide comprises the amino acid sequence of KASFTTFTVTKGS-NH2 (SEQ ID NO: 5).

16. The method of claim 1, wherein the modified Cav-1 peptide comprises the amino acid sequence of aaEGKASFTTFTVTKGSaa (SEQ ID NO: 6).

17. The method of claim 1, wherein the modified Cav-1 peptide comprises the amino acid sequence of aaEGKASFTTFTVTKGSaa-NH2 (SEQ ID NO: 7).

18. The method of claim 1, wherein the modified Cav-1 peptide comprises the amino acid sequence of Ac-aaEGKASFTTFTVTKGSaa-NH2 (SEQ ID NO: 8).

19. The method of claim 1, wherein the modified Cav-1 peptide comprises the amino acid sequence of OASFTTFTVTOS (SEQ ID NO: 9).

20. The method of claim 1, wherein the modified Cav-1 peptide comprises the amino acid sequence of OASFTTFTVTOS-NH2 (SEQ ID NO: 10).

21. The method of any one of claims 1-20, wherein the modified Cav-1 peptide comprises an internalization sequence.

22. The method of claim 21, wherein the internalization sequence is located at the C-terminal end of the peptide.

23. The method of claim 21, wherein the internalization sequence is located at the N-terminal end of the peptide.

24. The method of any one of claims 1-23, wherein the modified Cav-1 peptide further comprises a cap at the N- and/or C-terminus.

25. The method of claim 24, wherein the modified Cav-1 peptide comprises the cap at the N-terminus and C-terminus.

26. The method of any one of claims 1-25, wherein the modified Cav-1 peptide is cyclized.

27. The method of any one of claims 1-26, wherein the modified Cav-1 peptide maintains the biological activity of native Cav-1 (SEQ ID NO: 1).

28. The method of claim 1, wherein the modified Cav-1 peptide is a multimer comprising at least two peptides of any one of claims 1-25.

29. The method of claim 28, wherein a first peptide of the at least two peptides is essentially identical to a second peptide of the at least two peptides.

30. The method of claim 28, wherein a first peptide of the at least two peptides is not identical to a second peptide of the at least two peptides.

31. The method of any one of claims 1-30, wherein the modified Cav-1 peptide is administered intravenously, subcutaneously, intramuscularly, intraperitoneally, or orally.

32. The method of claim 31, wherein the modified Cav-1 peptide is administered subcutaneously.

33. The method of claim 31, wherein the modified Cav-1 peptide is administered intravenously.

34. The method of any one of claims 1-33, wherein the kidney disease or disorder is selected from the group consisting of chronic kidney disease, end-stage renal disease, glomerulonephritis, focal segmental glomerulosclerosis, kidney fibrosis, polycystic kidney disease, IgA nephropathy, lupus nephritis, nephrotic syndrome, Alport syndrome, amyloidosis, Goodpasture syndrome, granulomatosis with polyangiitis, or acute kidney injury.

35. The method of claim 34, wherein the kidney disease or disorder is Alport syndrome.

36. The method of any one of claims 1-33, wherein the kidney disease or disorder is characterized by fibrosis.

37. The method of any one of claims 1-36, wherein the method further comprises administering an effective amount of at least one additional therapeutic agent.

38. The method of claim 37, wherein the at least one additional therapeutic agent is an angiotensin-converting enzyme (ACE) inhibitor and/or angiotensin II receptor (ARB) inhibitor.

39. The method of any one of claims 1-38, wherein the method further comprises treating the subject with dialysis.

40. The method of any one of claims 1-39, wherein the modified Cav-1 peptide is administered as a composition comprising the modified Cav-1 peptide and at least one pharmaceutically acceptable carrier or excipient.

41. A method of treating or preventing a fibrotic disease or disorder in an elderly subject comprising administering to the subject an effective amount of a modified Cav-1 peptide:
(d) consisting of any one of the amino acid sequences of SEQ ID NOs: 2-111;
(e) comprising any one of the amino acid sequences of SEQ ID NOs: 2-111; or
(f) comprising any one of the amino acid sequences of SEQ ID NOs: 2-111 with one or more amino acid substitutions, insertions, deletions, or chemical modifications.

42. The method of claim 41, wherein the modified Cav-1 peptide comprises the amino acid sequence of FTTFTVT (SEQ ID NO: 3).

43. The method of claim 41, wherein the modified Cav-1 peptide comprises the amino acid sequence of Ac-aaEGKASFTTFTVTKGSaa-NH2 (SEQ ID NO: 8).

44. The method of any one of claims 41-43, wherein the fibrotic disease or disorder is interstitial lung disease.

45. The method of claim 44, wherein the interstitial lung disease is idiopathic pulmonary fibrosis.

46. The method of any one of claims 41-45, wherein the modified Cav-1 peptide is administered to the lung.

47. The method of claim 46, wherein the modified Cav-1 peptide is administered to the lung via inhalation.

48. The method of any one of claims 41-47, wherein the modified Cav-1 peptide is formulated for inhalation.

49. The method of claim 48, wherein the modified Cav-1 peptide is formulated for pressurized metered dose inhalation.

50. The method of claim 48, wherein the modified Cav-1 peptide is formulated as a dry powder.

51. The method of claim 50, wherein the dry powder comprising the modified Cav-1 peptide is essentially excipient free.

52. The method of claim 50 or 51, wherein the dry powder is produced by a spray-drying process, air jet milling, ball milling, or wet milling.

53. The method of claim 48, wherein the modified Cav-1 peptide is formulated for nebulization.

54. The method of claim 47, wherein the modified Cav-1 peptide is administered to the subject using a nebulizer.

55. The method of claim 47, wherein the modified Cav-1 peptide is administered to the subject using an inhaler.

56. The method of any one of claims 41-55, wherein the method further comprises administering to the subject a therapeutically effective amount of at least one additional therapeutic agent.

57. The method of claim 56, wherein the at least one additional therapeutic agent is chloroquine, hydroxychloroquine, remdesivir, favipiravir, lopinavir, or ritonavir.

58. The method of any one of claims 41-57, wherein the subject is at least 55 years old, at least 60 years old, at least 65 years old, at least 70 years old, at least 75 years old, at least 80 years old, at least 85 years old, or at least 90 years old.
